SEQUENCE LISTING (10 sequences)

SEQ ID NO: 1 - protein, 6 aa, Artificial Sequence
  Antibody
QFVGSS

SEQ ID NO: 2 - protein, 3 aa, Artificial Sequence
  Antibody
YAS

SEQ ID NO: 3 - protein, 9 aa, Artificial Sequence
  Antibody
QQSHSWPFT

SEQ ID NO: 4 - protein, 8 aa, Artificial Sequence
  Antibody
GFIFSNHW

SEQ ID NO: 5 - protein, 10 aa, Artificial Sequence
  Antibody
IRSKSINSAT

SEQ ID NO: 6 - protein, 11 aa, Artificial Sequence
  Antibody
SRNYYGSTYDY

SEQ ID NO: 7 - protein, 107 aa, Artificial Sequence
  Antibody
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 8 - protein, 119 aa, Artificial Sequence
  Antibody
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 9 - protein, 236 aa, Artificial Sequence
  Antibody
MDFQVQIISFLLISASVIMSRGDILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 119 aa, Artificial Sequence; identical to SEQ ID NO: 8
  Antibody

(19) United States
(12) Patent Application Publication
(10) Pub. No.: US 2022/0153828 A1
(43) Pub. Date: May 19, 2022

(54) Methods for Treating TNFa-Related Diseases

(71) Applicant: Celltrion Inc., Incheon (KR)

(72) Inventors: Sun Jung Kim, Incheon (KR); Sera Kim, Incheon (KR); Jee Hye Suh, Incheon (KR); Si Young Yang, Incheon (KR); Joon Ho Lee, Incheon (KR); So Hye Jo, Incheon (KR); Jin Sun Jung, Incheon (KR); Sun Hee Lee, Incheon (KR)

(21) Appl. No.: 17/430,628

(22) PCT Filed: Feb. 28, 2020
(86) PCT No.: PCT/KR2020/002886

§ 371 Date: Aug. 12, 2021

(30) Foreign Application Priority Data
Feb. 28, 2019 (KR) 10-2019-0023769

(51) Int. Cl. C07K 16/24

(52) U.S. Cl. CPC C07K 16/241; A61K 2039/505

(57) ABSTRACT

The present prevention relates to methods for treating TNFα-related diseases by subcutaneously administering an antibody binding to TNFα (anti-TNFα antibody) or an antigen-binding fragment thereof. The treatment method, composition, kit or use according to the present invention provide an advantage of improving patient satisfaction, by improving convenience and quality of life, that is, by reducing the time required for administration and decreasing the length of stay of patients in a hospital compared to intravenous injection.

### TECHNICAL FIELD

The present disclosure relates to methods for treating TNFα-related diseases by subcutaneously administering an antibody binding to TNFα (anti-TNFα antibody).

### BACKGROUND ART

Tumor necrosis factor α (TNFα) is a cell signaling protein (cytokine) which is involved in systemic inflammations, and is one of the cytokines which form acute phase responses. TNFα is associated with a variety of diseases and disorders including sepsis, infections, autoimmune diseases and graft rejection. TNFα stimulates immune responses which cause a number of clinical problems associated with autoimmune abnormalities such as rheumatoid arthritis, ankylosing spondylitis, ulcerative colitis, adult Crohn's disease, pediatric Crohn's disease, psoriasis, psoriatic arthritis, etc. Such abnormalities may be treated by using TNFα inhibitors.

Infliximab is a type of chimeric monoclonal antibody capable of acting as the TNFα inhibitor, and now commercially available infliximab products include Remsima, Remicade, Renflexis, etc. However, these products are all prepared as lyophilized powders, which are reconstituted and diluted to be intravenously injected in accordance with a dosage regimen and dose selected for each disease.

However, the intravenous administration method as described above requires a patient to visit a hospital for medication and takes two to four hours including a waiting time, indicating that such method poses a considerable burden and inconvenience in daily life. In addition, there is a problem that a person who administers drugs is limited to a person who has received a medical education.

Thus, subcutaneous (SC) administration is proposed as an alternative route of administration. Such administration may allow a patient to perform a self-injection after training and the time required may be shortened to 2-5 minutes, while intravenous administration used to take 30-90 minutes in the prior art.

Commercially available formulation products developed not only for intravenous administration but also for subcutaneous administration, include Rituxan (Rituximab), Simponi (Golimumab), Herceptin (Trastuzumab), Actemra (Tocilizumab), Xolair (Omalizumab), etc, but a formulation for subcutaneous administration of infliximab has not been reported yet.

For subcutaneous administration, a stable liquid formulation containing a high concentration of antibody is required, and the clinical efficacy and safety thereof have to be demonstrated.

The present applicants have demonstrated that an CT-P13 formulation for subcutaneous administration has the same efficacy and stability as those of conventional formulations for intravenous administration, thereby completing a subcutaneous administration therapy that helps patients have more convenience of administration and improve their quality of life.

### DETAILED DESCRIPTION OF THE INVENTION

### Technical Problem

An object of the present invention is to provide a treatment method comprising subcutaneously administering to a subject a pharmaceutical composition containing an anti-TNFα antibody or an antigen-binding fragment thereof for treating TNFα-related diseases.

Another object of the present invention is to provide a pharmaceutical composition for treating diseases treatable with an anti-TNFα antibody, which contains the anti-TNFα antibody or an antigen-binding fragment thereof and is to be subcutaneously administered to a subject.

Yet another object of the present invention is to provide a kit comprising: a pharmaceutical composition containing an anti-TNFα antibody or an antigen-binding fragment thereof; and instructions that direct the pharmaceutical composition to be subcutaneously administered to a subject in order to treat diseases treatable with the anti-TNFα antibody.

Still yet another object of the present invention is to provide a use of an anti-TNFα antibody or an antigen-binding fragment thereof in preparing a drug which is to be subcutaneously administered to a subject in order to treat diseases treatable with the anti-TNFα antibody.

### Technical Solution

The present invention provides a method for treating diseases treatable with an anti-TNFα antibody, the method comprising a step of subcutaneously administering to a subject a pharmaceutical composition containing an anti-TNFα antibody or an antigen-binding fragment thereof.

Further, the present invention provides a pharmaceutical composition for treating diseases treatable with an anti-TNFα antibody, which contains the anti-TNFα antibody or an antigen-binding fragment thereof and is to be subcutaneously administered to a subject.

In addition, the present invention provides a kit comprising: (a) a pharmaceutical composition containing an anti-TNFα antibody or an antigen-binding fragment thereof and pharmaceutically acceptable carriers; and (b) instructions that direct the pharmaceutical composition to be subcutaneously administered to a subject in order to treat diseases treatable with the anti-TNFα antibody.

Besides, the present invention provides a use of an anti-TNFα antibody or an antigen-binding fragment thereof in preparing a pharmaceutical composition which is to be subcutaneously administered to a subject in order to treat diseases treatable with the anti-TNFα antibody.

In one embodiment of the present invention, the anti-TNFα antibody may comprise one or more selected from the group consisting of infliximab, adalimumab, certolizumab pegol, golimumab, and biosimilar thereof.

In one embodiment of the present invention, the anti-TNFα antibody may be infliximab.

In one embodiment of the present invention, the anti-TNFα antibody may comprise a chimeric human-mouse IgG monoclonal antibody.

In one embodiment of the present invention, the anti-TNFα antibody may comprise: a light-chain variable region comprising a CDR1 domain comprising an amino acid sequence of SEQ ID NO: 1, a CDR2 domain comprising an amino acid sequence of SEQ ID NO: 2, and a CDR3 domain comprising an amino acid sequence of SEQ ID NO: 3; and a heavy-chain variable region comprising a CDR1 domain comprising an amino acid sequence of SEQ ID NO: 4, a CDR2 domain comprising an amino acid sequence of SEQ ID NO: 5, and a CDR3 domain comprising an amino acid sequence of SEQ ID NO: 6.

In one embodiment of the present invention, the anti-TNFα antibody may comprise: a light-chain variable region comprising an amino acid sequence of SEQ ID NO: 7; and a heavy-chain variable region comprising an amino acid sequence of SEQ ID NO: 8.

In one embodiment of the present invention, the anti-TNFα antibody may comprise: a light chain comprising an amino acid sequence of SEQ ID NO: 9; and a heavy chain comprising an amino acid sequence of SEQ ID NO: 10.

In one embodiment of the present invention, the composition may comprise: a surfactant; a sugar or derivatives thereof; and a buffer comprising acetate or histidine.

In one embodiment of the present invention, the composition may comprise polysorbate 20, polysorbate 40, polysorbate 60, polysorbate 80, or a mixture thereof as the surfactant.

In one embodiment of the present invention, the concentration of the surfactant in the composition may be 0.02 to 0.1% (w/v).

In one embodiment of the present invention, the composition may comprise sorbitol, mannitol, trehalose, sucrose, or a mixture thereof as the sugar or derivatives thereof.

In one embodiment of the present invention, the concentration of the sugar or derivatives thereof in the composition may be 1 to 10% (w/v).

In one embodiment of the present invention, the composition may comprise acetate as the buffer.

In one embodiment of the present invention, the concentration of the buffer in the composition may be 1 to 50 mM.

In one embodiment of the present invention, the composition may have a pH of 4.0 to 5.5.

In one embodiment of the present invention, the composition may comprise: (A) 90 to 180 mg/ml of the anti-TNFα antibody; (B) 0.02 to 0.1% (w/v) of polysorbate; (C) 1 to 10% (w/v) of sorbitol; and (D) 1 to 50 mM of a buffer comprising acetate or histidine.

In one embodiment of the present invention, the composition may be free of aspartic acid, lysine, arginine, or a mixture thereof.

In one embodiment of the present invention, the composition may be free of NaCl, KCl, NaF, KBr, NaBr, Na₂SO₄, NaSCN, K₂SO₄, or a mixture thereof.

In one embodiment of the present invention, the composition may be free of a chelating agent.

In one embodiment of the present invention, the composition may have a viscosity of 0.5 to 10.0 cp after one month of storage at a temperature of 40±2° C., or a viscosity of 0.5 to 5.0 cp after six months of storage at a temperature of 5±3° C.

In one embodiment of the present invention, the composition may not be subjected to a reconstitution step, a dilution step or both thereof before use.

In one embodiment of the present invention, the composition may be filled into a pre-filled syringe or an auto-injector before being administered to a subject.

In one embodiment of the present invention, the subject may comprise mammals.

In one embodiment of the present invention, the subject may comprise human beings.

In one embodiment of the present invention, the antibody or the antigen-binding fragment thereof may be administered at a dose of 60 to 300 mg.

In one embodiment of the present invention, the diseases treatable with the anti-TNFα antibody may comprise rheumatoid arthritis, ulcerative colitis, Crohn's disease, plaque psoriasis, psoriatic arthritis and ankylosing spondylitis.

In one embodiment of the present invention, the antibody or the antigen-binding fragment thereof may be administered at a dose of 60, 70, 80, 90, 100, 110, 120, 130, 140, 150, 160, 170, 180, 190, 200, 210, 220, 230, 240, 250, 260, 270, 280, 290 or 300 mg.

In one embodiment of the present invention, the antibody or the antigen-binding fragment thereof may be administered at a dose of 90 to 300 mg.

In one embodiment of the present invention, the antibody or the antigen-binding fragment thereof may be administered at a dose of 90 to 180 mg.

In one embodiment of the present invention, the antibody or the antigen-binding fragment thereof may be administered at a dose of 120 to 240 mg. In one embodiment of the present invention, the antibody or the antigen-binding fragment thereof may be administered at a dose of 80 to 100 mg, 110 to 130 mg, 170 to 190 mg, or 230 to 250 mg.

In one embodiment of the present invention, the antibody or the antigen-binding fragment thereof may be administered at a dose of 90, 120, 180 or 240 mg.

In one embodiment of the present invention, the anti-TNFα antibody or the antigen-binding fragment thereof may be administered to a patient at a dose of 90 to 180 mg, if TNFα-related disease is rheumatoid arthritis.

In one embodiment of the present invention, the anti-TNFα antibody or the antigen-binding fragment thereof may be administered to the patient at a dose of 90, 120 or 180 mg, if TNFα-related disease is rheumatoid arthritis.

In one embodiment of the present invention, the anti-TNFα antibody or the antigen-binding fragment thereof may be administered to the patient at a dose of 120 to 240 mg, if TNFα-related disease is one or more selected from the group consisting of ulcerative colitis, Crohn's disease, plaque psoriasis, psoriatic arthritis and ankylosing spondylitis.

In one embodiment of the present invention, the anti-TNFα antibody or the antigen-binding fragment thereof may be administered to the patient at a dose of 120, 150, 180 or 240 mg, if TNFα-related disease is one or more selected from the group consisting of ulcerative colitis, Crohn's disease, plaque psoriasis, psoriatic arthritis and ankylosing spondylitis. In one embodiment of the present invention, the antibody or the antigen-binding fragment thereof may be administered at an increased dose depending on the patient's condition.

In one embodiment of the present invention, the antibody or the antigen-binding fragment thereof may be administered at a dose of 90 to 180 mg, if the patient's body weight is less than 80 kg, and may be administered at a dose of 190 to 270 mg, if the patient's body weight is 80 kg or more.

In one embodiment of the present invention, the antibody or the antigen-binding fragment thereof may be administered at intervals of 1 to 8 weeks.

In one embodiment of the present invention, the antibody or the antigen-binding fragment thereof may be administered at intervals of 1, 2, 3, 4, 5, 6, 7 or 8 weeks.

In one embodiment of the present invention, the antibody or the antigen-binding fragment thereof may be administered at intervals of 2 or 4 weeks.

In one embodiment of the present invention, the patients to be dosed with the anti-TNFα antibody may exhibit one or more characteristics selected from the followings:

a) A patient who has an inadequate response to disease-modifying anti-rheumatic drugs (DMARDs) comprising methotrexate;

b) A patient who has not previously been treated with methotrexate and other DMARDs;

c) A patient who exhibits a rise in serologic indicators associated with severe axial symptoms and inflammation, which show no proper response to common therapies;

d) A patient who does not respond to, is contraindicated from, or has intolerance to methotrexate, cyclosporine, or systemic therapies comprising dermatologic photochemotherapy (psoralen ultraviolet A therapy: PUVA);

e) A patient who has no adequate response to treatment with corticosteroids, 6-mercaptopurine, azathioprine or immunosuppressants, or has intolerance to such therapy or is contraindicated from such treatment method; or

f) A patient who does not respond to common therapies, comprising antibiotic, excretion or immunosuppressive therapies.

In one embodiment of the present invention, the patient may be a patient who has been intravenously administered with the anti-TNFα antibody or the antigen-binding fragment thereof at least once prior to subcutaneous administration.

In one embodiment of the present invention, the patient may be a patient who has been intravenously administered with the anti-TNFα antibody or the antigen-binding fragment thereof twice or three times prior to subcutaneous administration.

In one embodiment of the present invention, a) the patient who has a rheumatoid arthritis disease may be a patient who has been intravenously administered with the anti-TNFα antibody or the antigen-binding fragment thereof twice prior to subcutaneous administration, and b) the patient who has one or more diseases selected from the group consisting of ulcerative colitis, Crohn's disease, plaque psoriasis, psoriatic arthritis and ankylosing spondylitis may be a patient who has been intravenously administered with the anti-TNFα antibody or the antigen-binding fragment thereof twice or three times prior to subcutaneous administration.

In one embodiment of the present invention, the patient may be a patient who has been intravenously administered with the anti-TNFα antibody or the antigen-binding fragment thereof twice at Weeks 0 and 2, or a patient who has been intravenously administered with the same three times at Weeks 0, 2 and 6 prior to subcutaneous administration.

In one embodiment of the present invention, the patient may be a patient who has been intravenously administered with the anti-TNFα antibody or the antigen-binding fragment thereof at least once prior to subcutaneous administration.

In one embodiment of the present invention, the patient may be a patient who has been intravenously administered with the anti-TNFα antibody or the antigen-binding fragment thereof at a dose of 1 to 10 mg/kg per administration prior to subcutaneous administration.

In one embodiment of the present invention, the patient may be a patient who has been intravenously administered with the anti-TNFα antibody or the antigen-binding fragment thereof at a dose of 3 to 5 mg/kg per administration prior to subcutaneous administration.

In one embodiment of the present invention, a) the patient who has a rheumatoid arthritis disease may be a patient who has been intravenously administered with the anti-TNFα antibody or the antigen-binding fragment thereof at a dose of 3 mg/kg per administration, and b) the patient who has one or more diseases selected from the group consisting of ulcerative colitis, Crohn's disease, plaque psoriasis, psoriatic arthritis and ankylosing spondylitis may be a patient who has been intravenously administered with the anti-TNFα antibody or the antigen-binding fragment thereof at a dose of 5 mg/kg per administration.

In one embodiment of the present invention, the first subcutaneous administration may be performed in 2 to 8 weeks after the last intravenous administration.

In one embodiment of the present invention, the first subcutaneous administration may be performed in 4 weeks after the last intravenous administration.

In one embodiment of the present invention, the composition containing the anti-TNFα antibody or the antigen-binding fragment thereof may be administered simultaneously with, before or after administration of one or more selected from the group consisting of infliximab, adalimumab, certolizumab pegol, golimumab, and biosimilar thereof.

In one embodiment of the present invention, the anti-TNFα antibody or the antigen-binding fragment thereof may be administered simultaneously with, before or after administration of one or more selected from the group consisting of anti-rheumatic drugs (DMARDs), steroids and immunosuppressants. Specifically, the disease-modifying anti-rheumatic drugs (DMARDs) may be selected from the group consisting of methotrexate, leflunomide, sulfasalazine and hydroxychloroquine, the steroids may be selected from the group consisting of corticosteroid, glucocorticoid, cortisol, mineralocorticoid and aldosterone, and the immunosuppressants may be selected from the group consisting of azathioprine, 6-mercaptopurine, cyclosporin A, tacrolimus, mycophenolic acid, bredinin, mTOR inhibitor and anti-lymphocyte antibody.

In one embodiment of the present invention, there may be provided an administration method in which a minimum serum concentration (C_{trough}; minimum concentration immediately before the next application) of the anti-TNFα antibody or the antigen-binding fragment thereof is maintained at 0.01 μg/ml or more after subcutaneous administration to the patient.

In one embodiment of the present invention, there may be provided an administration method in which a) a minimum serum concentration (C_{trough}) of the anti-TNFα antibody or the antigen-binding fragment thereof is maintained at 1 μg/ml or more for the patient who has a rheumatoid arthritis disease, and b) a minimum serum concentration (C_{trough}) of the anti-TNFα antibody or the antigen-binding fragment thereof is maintained at 5 μg/ml or more for the patient who has one or more diseases selected from the group consisting of ulcerative colitis, Crohn's disease, plaque psoriasis, psoriatic arthritis and ankylosing spondylitis.

In one embodiment of the present invention, the patient after subcutaneous administration may exhibit one or more characteristics selected from the followings:

a) a decrease in DAS28 (Disease Activity Score in 28 joints) by at least 2.0; or

b) a decrease in CDAI (Crohn's disease activity index) by at least 70.

### BRIEF DESCRIPTION OF THE DRAWINGS

FIG. 1 is a simulation graph showing a mean (±SD) of infliximab concentrations in blood over time when infliximab (IV or SC) is administered to CD patients in Study 1.6 Part 1 (A: Cohort dosed with SC 120 mg, B: Cohort dosed with SC 180 mg, and C: Cohort dosed with SC 240 mg).

FIG. 2 is a simulation graph showing a median of infliximab concentrations in blood over time at steady state when 120 mg of infliximab SC or IV is administered to inflammatory bowel disease (IBD) patients in Study 1.6 Part 2.

FIG. 3 is a graph showing a pharmacokinetic profile between IV and SC dosage forms of infliximab for 54 weeks (∘: SC dosage form and Δ: IV dosage form).

FIG. 4 is a simulation graph showing a median by time with regard to a method for administration of infliximab SC every 2 weeks from week 0 without administration of infliximab IV (A: Simulation graph of plasmatic concentrations of infliximab by time with regard to each experimental cohort (solid line: cohort dosed with SC 120 mg after 2 administrations of IV 3 mg/kg, and dotted line: cohort dosed with SC 120 mg), and B: Simulation cohort of DAS28 by time with regard to each experimental cohort).

FIG. 5 is a graph showing a minimum serum concentration (C_{trough}) boxplot (A) and a DAS28 score boxplot (B) at Weeks 2, 6 and 14 with regard to each experimental group (Gray box: Group dosed with SC 120 mg after two IV administrations and Red box: Group dosed with SC 120 mg).

FIG. 6 is a graph showing a pharmacokinetic profile between IV and SC dosage forms of infliximab for 54 weeks (●: SC dosage form and Δ: IV dosage form).

FIG. 7 is a graph comparing CDAI scores observed (indicated by ∘) and model-predicted CDAI scores (black solid line) with each other as a result of VPC obtained from a final PK-PD model.

FIG. 8 is a graph showing simulation data on average plasmatic concentrations by time with regard to each administration therapy from week 10 after the administration of IV 5 mg/kg to CD patients at weeks 0, 2 and 6.

FIG. 9 is a graph showing simulation data for CD patients on CDAI scores by time with regard to each administration therapy from week 10 after the administration of IV 5 mg/kg at weeks 0, 2 and 6.

FIG. 10 is a graph showing simulation data for UC patients on average plasmatic concentrations by time with regard to each administration therapy from week 10 after the administration of IV 5 mg/kg at weeks 0, 2 and 6.

FIG. 11 is a graph showing simulation data for UC patients on Mayo scores by time with regard to each administration therapy from week 10 after the administration of IV 5 mg/kg at weeks 0, 2 and 6.

### ADVANTAGEOUS EFFECTS

The treatment method, composition, kit or use according to the present invention makes it possible to treat TNFα-related diseases by subcutaneously administering an anti-TNFα antibody or an antigen-binding fragment thereof. In addition, the treatment method, composition, kit or use according to the present invention provides an advantage of improving patient satisfaction, by improving convenience and quality of life, that is, by reducing the time required for administration and decreasing the length of stay of patients in a hospital compared to intravenous injection.

Besides, the treatment method, composition, kit or use according to the present invention is added as a new treatment option of infliximab, thus providing an advantage of removing the burden and rejection caused by drug changes from patients who have been dosed with conventional infliximab via intravenous injection, as well as health care workers.

### MODE FOR INVENTION

The present invention relates to a method for treating diseases treatable with an anti-TNFα antibody, the method comprising a step of subcutaneously administering to a subject a pharmaceutical composition containing the anti-TNFα antibody or an antigen-binding fragment thereof.

To facilitate the understanding of the present invention, the terms used in the present invention are defined as follows.

“TNFα” is intended to refer to a human cytokine that exists as a 17 kD secreted form and a 26 kD membrane associated form, the biologically active form of which is composed of a trimer noncovalently bound to 17 kD molecules. The structure of TNFα is further described, for example, in documents (See Pennica, D., et al. (1984) Nature 312:724-729; Davis, J. M., et al. (1987) Biochemistry 26:1322-1326; and Jones, E. Y., et al. (1989) Nature 338:225-228).

The term “antibody” refers to an immunoglobulin molecule composed of four polypeptide chains, in which two heavy chains and two light chains are inter-connected by disulfide bonds. Other naturally occurring antibodies having an altered structure, for example, a camelid antibody, are also included in this definition. Each heavy chain is composed of a heavy-chain variable region and a heavy-chain constant region. The heavy-chain constant region is composed of three domains (CH1, CH2 and CH3). Each light chain is comprised of a light-chain variable region and a light-chain constant region. The light-chain constant region is comprised of one domain (CL). The heavy-chain variable region and the light-chain variable region may be further subdivided into regions of hypervariability, termed complementarity determining regions (CDR), which are interspersed with more conserved regions, termed framework regions (FR). Each of the heavy-chain variable region and the light-chain variable region is composed of three CDRs and four FRs, which are arranged from amino-terminus to carboxy-terminus in the following order: FR1, CDR1, FR2, CDR2, FR3, CDR3 and FR4.

The term “antigen-binding fragment” refers to one or more fragments of an antibody, which retain the ability to specifically bind to an antigen bound by the complete antibody. An exemplary antigen-binding fragment includes, but is not limited to, Fab, Fab′, F(ab′)₂, Fv, and the like.

The term “biosimilar” means a biological product which is highly similar to an FDA-approved biological product (reference drug) and has no clinically meaningful difference from the reference product in terms of pharmacokinetics, safety and efficacy.

The term “biological formulation” or “biological product” refers to a medicinal product that is prepared with raw materials or substances derived from human or other living organisms and that requires special attentions for public health, and includes biologics, recombinant DNA products, cell culture-derived products, cell therapy products, gene therapy products, and other formulations approved by the Minister of Food and Drug Safety.

The term “administration” refers to administration of a substance (e.g., anti-TNFα antibody) for achieving therapeutic purposes (e.g., TNFα-related disease).

The term “TNFα-related disease” refers to a local and/or systemic physiological disease in which TNFα is a primary mediator leading to the manifestation of the disease. The terms “TNFα-related disease,” “disease treatable with anti-TNFα” and “disease where the activity of TNFα is harmful” are used interchangeably herein.

The term “subject” includes all humans or non-human animals. The term “non-human animals” include, but are not limited to, vertebrates such as non-human primates, sheep, dogs, cats, rabbits and ferrets, rodents such as mice, rats and guinea pigs, and bird species such as chickens, amphibians, and reptiles. In a preferred embodiment aspect, the subject is mammals such as non-human primates, sheep, dogs, cats, rabbits, ferrets, or rodents. In a more preferred embodiment aspect, the subject is a human being. The terms “subject,” “patient” and “individual” are used interchangeably herein.

The term “IC50” is intended to refer to the concentration of an inhibitor, which is required to inhibit the biological outcome of interest, for example, to neutralize cytotoxic activity.

The term “minimum serum concentration (C_{trough}),” which is an abbreviation for model-predicted trough serum concentration, means the minimum concentration of a drug in blood, predicted by using a population pharmacokinetic model.

The term “DAS28 (disease activity score in 28 joints)” refers to a method for evaluating disease activity in rheumatoid arthritis (RA) using 28 joints.

The term “CDAI (Crohn's disease activity index)” refers to a study tool used in quantifying symptoms of patients with Crohn's disease.

The term “anti-rheumatic drug (disease-modifying anti-rheumatic drug, DMARD)” refers to a combination of oral drugs, which are effective in alleviating symptoms of arthritis and delaying progression of the disease. DMARD prevents an immune system from having an effect of releasing chemical substances which attack joints and do damage to bones, tendons, ligaments or cartilages. Specific types of DMARD-based drug comprise methotrexate, hydroxychloroquine, sulfasalazine and leflunomide.

The term “kit” refers to a packaged product including components for administrating the TNFα antibody of the present invention to treat TNFα-related diseases. The kit preferably includes a container or box which holds the components of the kit. The box or container is affixed with a label or a protocol approved by the Food and Drug Administration. The box or container holds components of the present invention, which are contained in plastic, polyethylene, polypropylene, ethylene or propylene containers. The container may be a capped-tube or bottle. The kit also includes instructions for administering the TNFα antibody of the present invention.

Various aspects of the present invention will be described in further detail.

Anti-TNFα Antibody or Antigen-Binding Fragment Thereof According to the Present Invention

In one embodiment of the present invention, the antibody may comprise a polyclonal antibody, a monoclonal antibody, a recombinant antibody, a single-chain antibody, a hybrid antibody, a chimeric antibody, a humanized antibody, or a fragment thereof. The term “chimeric antibody” means an antibody comprising heavy-chain and light-chain variable region sequences from one species, and constant region sequences from another species. In one embodiment of the present invention, the antibody may comprise a chimeric human-mouse IgG monoclonal antibody. The chimeric human-mouse IgG monoclonal antibody is composed of mouse heavy-chain and light-chain variable regions and human heavy-chain and light-chain constant regions bound thereto. The chimeric human-mouse IgG monoclonal antibody may be prepared according to a method known in the art. For example, infliximab may be prepared according to a method described in U.S. Pat. No. 6,284,471.

In one embodiment of the present invention, the antibody may comprise an antibody which binds to TNFα or an epitope of TNFα. The antibody binding to TNFα or the epitope of TNFα may comprise one or more selected from the group consisting of infliximab, adalimumab, certolizumab pegol, golimumab, or biosimilar thereof. In one embodiment of the present invention, the antibody may comprise infliximab.

In one embodiment of the present invention, the antibody or the antigen-binding fragment thereof may comprise: a light-chain variable region comprising a CDR1 domain comprising an amino acid sequence of SEQ ID NO: 1, a CDR2 domain comprising an amino acid sequence of SEQ ID NO: 2, and a CDR3 domain comprising an amino acid sequence of SEQ ID NO: 3; and a heavy-chain variable region comprising a CDR1 domain comprising an amino acid sequence of SEQ ID NO: 4, a CDR2 domain comprising an amino acid sequence of SEQ ID NO: 5, and a CDR3 domain comprising an amino acid sequence of SEQ ID NO: 6.

In one embodiment of the present invention, the antibody or the antigen-binding fragment thereof may comprise: a light-chain variable region comprising an amino acid sequence of SEQ ID NO: 7; and a heavy-chain variable region comprising an amino acid sequence of SEQ ID NO: 8.

In one embodiment of the present invention, the antibody may comprise: a light chain comprising an amino acid sequence of SEQ ID NO: 9; and a heavy chain comprising an amino acid sequence of SEQ ID NO: 10.

Composition Containing Anti-TNFα Antibody or Antigen-Binding Fragment Thereof According to the Present Invention

As used herein, the term “composition containing an anti-TNFα antibody or an antigen-binding fragment thereof according to the present invention” is used interchangeably with a “stable liquid pharmaceutical formulation.”

The composition according to the present invention contains: (A) an antibody or an antigen-binding fragment thereof; (B) a surfactant; (C) a sugar or derivatives thereof; and (D) a buffer.

As used herein, the term “free of” means that the corresponding component is not contained at all. Further, the corresponding term means that the corresponding component is not substantially contained at all, that is, being contained within a range that does not affect the activity of the antibody and the stability and viscosity of the liquid pharmaceutical formulation. For example, the term means that the corresponding component is contained in an amount of 0 to 1% (w/v), 0 to 1 ppm (w/v), or 0 to 1 ppb (w/v), based on the total weight of the liquid pharmaceutical formulation.

(A) Antibody or Antigen-Binding Fragment Thereof

In one embodiment, the composition according to the present invention may comprise the inventive anti-TNFα antibody or the antigen-binding fragment thereof as described in detail above.

The concentration of the antibody or the antigen-binding fragment thereof may be freely controlled within a range that does not substantially adversely affect the stability and viscosity of the composition according to the present invention. In one embodiment of the present invention, the concentration of the antibody or the antigen-binding fragment thereof may be 10 to 200 mg/ml. In another embodiment of the present invention, the concentration of the antibody or the antigen-binding fragment thereof may be 50 to 200 mg/ml. In still another embodiment of the present invention, the concentration of the antibody or the antigen-binding fragment thereof may be 80 to 200 mg/ml. In still another embodiment of the present invention, the concentration of the antibody or the antigen-binding fragment thereof may be 90 to 180 mg/ml. In still another embodiment of the present invention, the concentration of the antibody or the antigen-binding fragment thereof may be 90 to 145 mg/ml. In still another embodiment of the present invention, the concentration of the antibody or the antigen-binding fragment thereof may be 110 to 130 mg/ml. If the concentration of the antibody or the antigen-binding fragment thereof is within the range above, the degree of freedom of administered dose and dosing cycle may be increased according to the high content of the antibody or the antigen-binding fragment thereof, and an excellent long-term stability and low viscosity may be exhibited.

(B) Surfactant

Examples of the surfactant comprise, but are not limited to, polyoxyethylene sorbitan fatty acid ester (e.g., polysorbate), polyoxyethylene alkyl ether (e.g., Brij), alkylphenyl polyoxyethylene ether (e.g., Triton-X), polyoxyethylene-polyoxypropylene copolymers (e.g., Poloxamer, Pluronic), sodium dodecyl sulfate (SDS), etc.

In one embodiment of the present invention, the surfactant may comprise polyoxyethylene sorbitan fatty acid ester (polysorbate). The polysorbate may comprise polysorbate 20, polysorbate 40, polysorbate 60, polysorbate 80, or a mixture of two or more thereof. In one embodiment of the present invention, the polysorbate may comprise polysorbate 20, polysorbate 80, or a mixture thereof. In another embodiment of the present invention, the polysorbate may comprise polysorbate 80.

In one embodiment of the present invention, the concentration of the surfactant may be freely controlled within a range that does not adversely affect the stability and viscosity of the stable liquid pharmaceutical formulation according to the present invention. For example, the concentration of the surfactant may be 0.001 to 5% (w/v), 0.01 to 1% (w/v), or 0.02 to 0.1% (w/v). If the concentration of the surfactant is within the range above, an excellent long-term stability and low viscosity may be exhibited.

(C) Sugar or Derivatives Thereof

The sugar may comprise a monosaccharide, a disaccharide, an oligosaccharide, a polysaccharide, or a mixture of two or more thereof. Examples of the monosaccharide comprise, but are not limited to, glucose, fructose, galactose, etc. Examples of the disaccharide comprise, but are not limited to, sucrose, lactose, maltose, trehalose, etc. Examples of the oligosaccharide comprise, but are not limited to, fructooligosaccharides, galactooligosaccharides, mannan oligosaccharides, etc. Examples of the polysaccharide comprise, but are not limited to, starch, glycogen, cellulose, chitin, pectin, etc.

The sugar derivatives may comprise sugar alcohol, sugar acid, or a mixture thereof. Examples of the sugar alcohol comprise, but are not limited to, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, etc. Examples of the sugar acid comprise, but are not limited to, aldonic acid (glyceric acid, etc.), ulosonic acid (neuraminic acid, etc.), uronic acid (glucuronic acid, etc.), aldaric acid (tartaric acid, etc.), etc.

In one embodiment of the present invention, the sugar or derivatives thereof may comprise sorbitol, mannitol, trehalose, sucrose, or a mixture of two or more thereof.

In one embodiment of the present invention, the concentration of the sugar or derivatives thereof may be freely controlled within a range that does not substantially adversely affect the stability and viscosity of the liquid pharmaceutical formulation according to the present invention. For example, the concentration of the sugar or derivatives thereof may be 0.1 to 30% (w/v), 1 to 20% (w/v), or 1 to 10% (w/v). If the concentration of the sugar or derivatives thereof may be within the range above, an excellent long-term stability and low viscosity may be exhibited.

(D) Buffer

The buffer is a neutralizing substance which minimizes a change in pH caused by acid or alkali. Examples of the buffer comprise phosphate, acetate, succinate, gluconate, glutamate, citrate, histidine, etc. In one embodiment of the present invention, the buffer may comprise acetate or histidine. If the buffer comprises both acetate and histidine, the stability may be reduced.

In one embodiment of the present invention, the buffer may comprise acetate. Examples of the acetate comprise, but are not limited to, sodium acetate, zinc acetate, aluminum acetate, ammonium acetate, potassium acetate, etc. For pH adjustment, the buffer may further comprise an acid, for example, acetic acid. Including acetate as the buffer may be most preferable in terms of pH adjustment and stability.

In one embodiment of the present invention, the buffer may comprise histidine. If histidine is used as the buffer, the histidine may comprise histidine salt, for example, histidine chloride, histidine acetate, histidine phosphate, histidine sulfate, etc. For pH adjustment, the buffer may comprise an acid, for example, hydrochloric acid, acetic acid, phosphoric acid, sulfuric acid, etc.

In one embodiment of the present invention, the stable liquid pharmaceutical formulation may be free of citrate, phosphate, or a mixture thereof.

In one embodiment of the present invention, the content of the buffer (or anions of the buffer) may be freely controlled within a range that does not substantially adversely affect the stability and viscosity of the liquid pharmaceutical formulation according to the present invention. For example, the content of the buffer or the anions thereof may be 1 to 50 mM, 5 to 30 mM, or 10 to 25 mM. If the content of the buffer or the anions thereof is within the range above, an excellent long-term stability and low viscosity may be exhibited.

(E) pH

In one embodiment of the present invention, the pH of the stable liquid pharmaceutical composition may be 4.0 to 5.5, or 4.7 to 5.3. If the pH is within the range above, an excellent long-term stability and low viscosity may be exhibited. The pH may be adjusted by using the buffer. In other words, if the buffer is contained in a pre-determined content, the pH may be exhibited in the range above without a need for a separate pH-adjusting agent. If citrate, phosphate or a mixture thereof is used as the buffer, it may be difficult to exhibit the pH in the range above. If an acid (e.g., hydrochloric acid) or a base (e.g., sodium hydroxide) is further contained as a separate pH-adjusting agent, the stability of the antibody may be reduced.

(F) Other Components

In one embodiment of the present invention, the stable liquid pharmaceutical formulation may be free of aspartic acid, lysine, arginine, or a mixture thereof. In case of containing these amino acids, such formulation may become a solid state. In one embodiment of the present invention, the stable liquid pharmaceutical formulation may contain one or more amino acids, excluding the three amino acids above. In this case, the amino acids may be contained in a range of 5% (w/v) or less, for example, in a range of 0.001 to 5% (w/v), in a range of 0.001 to 1% (w/v), in a range of 0.01 to 5% (w/v), in a range of 0.01 to 1% (w/v), in a range of 0.1 to 5% (w/v), or in a range of 0.1 to 1% (w/v).

In another embodiment of the present invention, the stable liquid pharmaceutical formulation may contain taurine. In this case, the taurine may be contained in a range of 5% (w/v) or less, for example, in a range of 0.001 to 5% (w/v), in a range of 0.001 to 1% (w/v), in a range of 0.01 to 5% (w/v), in a range of 0.01 to 1% (w/v), in a range of 0.1 to 5% (w/v), or in a range of 0.1 to 1% (w/v).

In one embodiment of the present invention, the stable liquid pharmaceutical formulation may be free of a metal salt, such as NaCl, KCl, NaF, KBr, NaBr, Na₂SO₄, NaSCN, K₂SO₄, etc. In case of containing these metal salts, a precipitation phenomenon may occur to the formulation, which may have a shape of gelatin and may have a poor stability.

In one embodiment of the present invention, the stable liquid pharmaceutical formulation may be free of a chelating agent (e.g., EDTA). In case of containing the chelating agent, an oxidation rate thereof may be increased.

In one embodiment of the present invention, the stable liquid pharmaceutical formulation may be free of a preservative. Examples of the preservative comprise octadecyl dimethyl benzyl ammonium chloride, hexamethonium chloride, benzalkonium chloride, benzethonium chloride, phenol, butyl alcohol, benzyl alcohol, alkyl paraben, catechol, resorcinol, cyclohexanol, 3-pentanol, m-cresol, etc. In case of containing the preservative, the preservative may not help improve the stability of the pharmaceutical formulation.

In one embodiment of the present invention, the stable liquid pharmaceutical formulation of the present invention may further comprise an additive known in the art within a range that does not substantially adversely affect the activity of the antibody and the stability and low viscosity of the formulation. For example, such pharmaceutical formulation may further comprise an aqueous carrier, an antioxidant, or a mixture of two or more thereof. The aqueous carrier is a carrier which is pharmaceutically acceptable (safe and non-toxic when administered to humans) and is useful for preparation of liquid pharmaceutical formulations. Examples of the aqueous carrier comprise, but are not limited to, sterile water for injection (SWFI), bacteriostatic water for injection (BWFI), sterile saline solution, Ringer's solution, dextrose, etc. Examples of the antioxidant comprise, but are not limited to, ascorbic acid, etc.

(G) “Stable” Liquid Pharmaceutical Formulation

In the “stable” liquid pharmaceutical formulation of the present invention, the term “stable” means that the antibody according to the present invention substantially retains its physical stability and/or chemical stability and/or biological activity during a production process and/or during preservation/storage. Various analytical techniques for measuring the stability of antibodies may be readily available in the art.

Physical stability may be evaluated by methods known in the art, which comprise measurement of a sample's apparent attenuation of light (absorbance or optical density). Such a measurement of light attenuation is related to the turbidity of a formulation. In addition, for physical stability, the content of high molecular weight components, the content of low molecular weight components, the amount of intact proteins, the number of sub-visible particles, etc., may be measured.

Chemical stability may be evaluated, for example, by detecting and quantifying chemically altered forms of the antibody. Chemical stability comprises, for example, a change in electric charges (e.g., occurring as a result of deamidation or oxidation), which may be evaluated, for example, by ion-exchange chromatography. For chemical stability, charge variants (acidic or basic peaks), etc., may be measured.

Biological activity may be evaluated by methods known in the art. For example, antigen-binding affinity may be measured by ELISA.

In one embodiment of the present invention, the liquid pharmaceutical formulation may be stable for a long period of time.

In one embodiment of the present invention, the term “stable” liquid pharmaceutical formulation means a liquid pharmaceutical formulation satisfying one or more of the followings:

Turbidity
a liquid pharmaceutical formulation having an absorbance A₆₀₀ of 0 to 0.0300 or 0 to 0.0700 as measured by a spectrophotometer after being stored for four weeks at a temperature of 40±2° C.;
a liquid pharmaceutical formulation having an absorbance A₆₀₀ of 0 to 0.0300 or 0 to 0.0700 as measured by a spectrophotometer after being stored for four weeks at a temperature of 40±2° C. and a relative humidity of 75±5% under a closed condition;

Content of Main Component (Main Peak)
a liquid pharmaceutical formulation in which the content of a main component is 98 to 100% as measured by SE-HPLC after being stored for four weeks at a temperature of 40±2° C.;
a liquid pharmaceutical formulation in which the content of a main component is 98 to 100% as measured by SE-HPLC after being stored for four weeks at a temperature of 40±2° C. and a relative humidity of 75±5% under a closed condition;

Content of High Molecular Weight Components (a Peak of which Retention Time is Earlier than that of the Main Peak (Intact IgG))
a liquid pharmaceutical formulation in which the content of high molecular weight components is 0 to 1.00% as measured by SE-HPLC after being stored for 12 months at a temperature of 5±3° C.;
a liquid pharmaceutical formulation in which the content of high molecular weight components is 0 to 1.00% as measured by SE-HPLC after being stored for 12 months at a temperature of 5±3° C. under a closed condition;

Content of Low Molecular Weight Components (a Peak of which Retention Time is Later than that of the Main Peak (Intact IgG))
a liquid pharmaceutical formulation in which the content of low molecular weight components is 0 to 0.40% as measured by SE-HPLC after being stored for 12 months at a temperature of 5±3° C.;
a liquid pharmaceutical formulation in which the content of low molecular weight components is 0 to 0.40% as measured by SE-HPLC after being stored for 12 months at a temperature of 5±3° C. under a closed condition;

Content of Intact Immunoglobulin G
a liquid pharmaceutical formulation in which the content of intact immunoglobulin G (intact IgG %) is 94.0 to 100% as measured by non-reducing CE-SDS after being stored for 12 months at a temperature of 5±3° C.;
a liquid pharmaceutical formulation in which the content of intact immunoglobulin G (intact IgG %) is 94.0 to 100% as measured by non-reducing CE-SDS after being stored for 12 months at a temperature of 5±3° C. under a closed condition;
a liquid pharmaceutical formulation in which the content of intact immunoglobulin G (intact IgG %) is 94.0 to 100% as measured by non-reducing CE-SDS after being stored for four weeks at a temperature of 40±2° C.;
a liquid pharmaceutical formulation in which the content of intact immunoglobulin G (intact IgG %) is 94.0 to 100% as measured by non-reducing CE-SDS after being stored for four weeks at a temperature of 40±2° C. and a relative humidity of 75±5% under a closed condition;

Content of Intact Heavy Chain and Light Chain
a liquid pharmaceutical formulation in which the content of intact heavy chain and light chain (intact HC+LC %) is 99.0 to 100% as measured by reducing CE-SDS after being stored for 12 months at a temperature of 5±3° C.;
a liquid pharmaceutical formulation in which the content of intact heavy chain and light chain (intact HC+LC %) is 99.0 to 100% as measured by reducing CE-SDS after being stored for 12 months at a temperature of 5±3° C. under a closed condition;
a liquid pharmaceutical formulation in which the content of intact heavy chain and light chain (intact HC+LC %) is 98.0 to 100% as measured by reducing CE-SDS after being stored for four weeks at a temperature of 40±2° C.;
a liquid pharmaceutical formulation in which the content of intact heavy chain and light chain (intact HC+LC %) is 98.0 to 100% as measured by reducing CE-SDS after being stored for four weeks at a temperature of 40±2° C. and a relative humidity of 75±5% under a closed condition;

Number of Sub-Visible Particles
a liquid pharmaceutical formulation in which the number of sub-visible particles (10.00 μm≤, <400.00 μm) is 0 to 1,000 as measured by HIAC after being stored for 12 months at a temperature of 5±3° C.;
a liquid pharmaceutical formulation in which the number of sub-visible particles (10.00 μm≤, <400.00 μm) is 0 to 1,000 as measured by HIAC after being stored for 12 months at a temperature of 5±3° C. under a closed condition;
a liquid pharmaceutical formulation in which the number of sub-visible particles (1.00 μm≤, <100.00 μm) is 0 to 30,000 as measured by MFI after being stored for four weeks at a temperature of 40±2° C.;
a liquid pharmaceutical formulation in which the number of sub-visible particles (1.00 μm≤, <100.00 μm) is 0 to 30,000 as measured by MFI after being stored for four weeks at a temperature of 40±2° C. and a relative humidity of 75±5% under a closed condition;
a liquid pharmaceutical formulation in which the number of sub-visible particles (10.00 μm≤, <100.00 μm) is 0 to 200 as measured by MFI after being stored for four weeks at a temperature of 40±2° C.;
a liquid pharmaceutical formulation in which the number of sub-visible particles (10.00 μm≤, <100.00 μm) is 0 to 200 as measured by MFI after being stored for four weeks at a temperature of 40±2° C. and a relative humidity of 75±5% under a closed condition;
a liquid pharmaceutical formulation in which the number of sub-visible particles (10.00 μm≤, <100.00 μm) is 0 to 500 as measured by MFI after being stored for six weeks at a temperature of 40±2° C.;
a liquid pharmaceutical formulation in which the number of sub-visible particles (10.00 μm≤, <100.00 μm) is 0 to 500 as measured by MFI after being stored for six weeks at a temperature of 40±2° C. and a relative humidity of 75±5% under a closed condition;

Oxidation Rate
a liquid pharmaceutical formulation in which an oxidation rate of heavy-chain Met 255 is 0 to 2.5% as measured by LC-MS after being stored for four weeks at a temperature of 40±2° C.;
a liquid pharmaceutical formulation in which the oxidation rate of heavy-chain Met 255 is 0 to 2.5% as measured by LC-MS after being stored for four weeks at a temperature of 40±2° C. and a relative humidity of 75±5% under a closed condition;

Charge Variants
a liquid pharmaceutical formulation in which an acidic peak is 20 to 35% as measured by IEC-HPLC after being stored for four weeks at a temperature of 40±2° C.;
a liquid pharmaceutical formulation in which an acidic peak is 20 to 35% as measured by IEC-HPLC after being stored for four weeks at a temperature of 40±2° C. and a relative humidity of 75±5% under a closed condition;
a liquid pharmaceutical formulation in which a basic peak is 33 to 40% as measured by IEC-HPLC after being stored for four weeks at a temperature of 40±2° C.;
a liquid pharmaceutical formulation in which a basic peak is 33 to 40% as measured by IEC-HPLC after being stored for four weeks at a temperature of 40±2° C. and a relative humidity of 75±5% under a closed condition;

TNFα Binding Affinity
a liquid pharmaceutical formulation in which a TNFα binding affinity is 80 to 120% as measured by ELISA after being stored for 12 months at a temperature of 5±3° C.; and
a liquid pharmaceutical formulation in which a TNFα binding affinity is 80 to 120% as measured by ELISA after being stored for 12 months at a temperature of 5±3° C. and under a closed condition.

In one embodiment of the present invention, the pharmaceutical formulation may have a viscosity of 0.5 to 10.0 cp as measured after being stored for one month at a temperature of 40±2° C. In another embodiment of the present invention, the pharmaceutical formulation may have a viscosity of 0.5 to 5.0 cp as measured after being stored for six months at a temperature of 5±3° C.

(H) Method for Preparing Stable Liquid Pharmaceutical Formulation

The stable liquid pharmaceutical formulation of the present invention may be prepared by using a known method, which is not limited to a particular method. For example, the liquid pharmaceutical formulation may be prepared by adding a buffer to a solution containing a surfactant and a sugar or derivatives thereof while adjusting a pH of the solution, and then adding an antibody to the resulting mixed solution. Further, the liquid pharmaceutical formulation may be prepared by preparing a solution containing some excipients in the final step of a purification process, and then adding the rest of components to the solution. For example, the liquid pharmaceutical formulation may be prepared by preparing a solution containing an antibody, a buffer and a sugar or derivatives thereof in the final step of the purification process, and then adding a surfactant to the solution.

In addition, the method for preparing the formulation may comprise a freeze-drying process or not.

If such preparation method does not comprise the freeze-drying process, for example, the liquid pharmaceutical formulation of the present invention may be prepared, then treated by sterilization, etc., and then immediately placed in a closed container.

If such preparation method comprises the freeze-drying process, for example, the liquid pharmaceutical formulation of the present invention may be prepared and freeze-dried, or the liquid pharmaceutical formulation of the present invention may be prepared, then freeze-dried, then preserved/stored, and then the components removed or modified by such freeze-drying and/or preservation/storage may be supplemented or replaced, thereby preparing the liquid pharmaceutical formulation according to the present invention. Alternatively, out of the liquid pharmaceutical formulation of the present invention, only the components excluding the components that may be removed or modified by freeze-drying and/or preservation/storage, may be freeze-dried, or such components may be freeze-dried and preserved/stored, and then the components excluded above may be added thereto, thereby preparing the liquid pharmaceutical formulation according to the present invention.

Korean Patent Application No. 10-2017-0081814 and Korean Patent Application No. 10-2018-0102233 previously filed by the present applicants are incorporated herein by reference.

Method for Treating Diseases Treatable with Anti-TNFα Antibody of the Present Invention

The present invention provides a method for treating diseases treatable with anti-TNFα, the method comprising a step of subcutaneously administering to a subject a pharmaceutical composition containing an anti-TNFα antibody or an antigen-binding fragment thereof.

In one embodiment of the present invention, the antibody may comprise one or more selected from the group consisting of infliximab, adalimumab, certolizumab pegol, golimumab, or biosimilar thereof.

In one embodiment of the present invention, the antibody may comprise infliximab.

In one embodiment of the present invention, the antibody may comprise a chimeric human-mouse IgG monoclonal antibody.

In one embodiment of the present invention, the antibody or the antigen-binding fragment thereof may comprise: a light-chain variable region comprising a CDR1 domain comprising an amino acid sequence of SEQ ID NO: 1, a CDR2 domain comprising an amino acid sequence of SEQ ID NO: 2, and a CDR3 domain comprising an amino acid sequence of SEQ ID NO: 3; and a heavy-chain variable region comprising a CDR1 domain comprising an amino acid sequence of SEQ ID NO: 4, a CDR2 domain comprising an amino acid sequence of SEQ ID NO: 5, and a CDR3 domain comprising an amino acid sequence of SEQ ID NO: 6.

In one embodiment of the present invention, the antibody or the antigen-binding fragment thereof may comprise: a light-chain variable region comprising an amino acid sequence of SEQ ID NO: 7; and a heavy-chain variable region comprising an amino acid sequence of SEQ ID NO: 8.

In one embodiment of the present invention, the antibody may comprise: a light chain comprising an amino acid sequence of SEQ ID NO: 9; and a heavy chain comprising an amino acid sequence of SEQ ID NO: 10.

In one embodiment of the present invention, the concentration of the antibody or the antigen-binding fragment thereof may be 10 to 200 mg/ml.

The present invention also provides a method for treating diseases treatable with anti-TNFα, the method comprising a step of subcutaneously administering to a subject a composition containing: (A) an anti-TNFα antibody or an antigen-binding fragment thereof; (B) a surfactant; (C) a sugar or derivatives thereof; and (D) a buffer.

In one embodiment of the present invention, the surfactant (B) may comprise polysorbate, poloxamer, or a mixture thereof.

In one embodiment of the present invention, the surfactant (B) may comprise polysorbate 20, polysorbate 40, polysorbate 60, polysorbate 80, or a mixture of two or more thereof.

In one embodiment of the present invention, the surfactant (B) may comprise polysorbate 80.

In one embodiment of the present invention, the concentration of the surfactant (B) may be 0.02 to 0.1% (w/v).

In one embodiment of the present invention, the sugar (C) may comprise a monosaccharide, a disaccharide, an oligosaccharide, a polysaccharide, or a mixture of two or more thereof, and the sugar derivative (C) may comprise sugar alcohol, sugar acid, or a mixture thereof.

In one embodiment of the present invention, the sugar or the derivatives thereof (C) may comprise sorbitol, mannitol, trehalose, sucrose, or a mixture of two or more thereof.

In one embodiment of the present invention, the concentration of the sugar or the derivatives thereof (C) may be 1 to 10% (w/v).

In one embodiment of the present invention, the buffer (D) may comprise acetate or histidine.

In one embodiment of the present invention, the content of the buffer (D) may be 1 to 50 mM.

In one embodiment of the present invention, the pH of the composition may be 4.0 to 5.5.

In one embodiment of the present invention, the composition may be free of aspartic acid, lysine, arginine, or a mixture thereof.

In one embodiment of the present invention, the composition may be free of NaCl, KCl, NaF, KBr, NaBr, Na₂SO₄, NaSCN, K₂SO₄ or a mixture thereof.

In one embodiment of the present invention, the composition may be free of a chelating agent.

In one embodiment of the present invention, the composition may be free of a preservative.

In one embodiment of the present invention, the composition may further contain an aqueous carrier, an antioxidant, or a mixture of two or more thereof.

In one embodiment of the present invention, the composition may have a viscosity of 0.5 to 10.0 cp as measured in one month later at a temperature of 40±2° C., or a viscosity of 0.5 to 5.0 cp as measured in six months later at a temperature of 5±3° C.

In one embodiment of the present invention, the composition may comprise: (A) an antibody or an antigen-binding fragment thereof, which comprises a light-chain variable region comprising a CDR1 domain comprising an amino acid sequence of SEQ ID NO: 1, a CDR2 domain comprising an amino acid sequence of SEQ ID NO: 2, and a CDR3 domain comprising an amino acid sequence of SEQ ID NO: 3; and a heavy-chain variable region comprising a CDR1 domain comprising an amino acid sequence of SEQ ID NO: 4, a CDR2 domain comprising an amino acid sequence of SEQ ID NO: 5, and a CDR3 domain comprising an amino acid sequence of SEQ ID NO: 6; (B) a surfactant; (C) a sugar or derivatives thereof; and (D) a buffer comprising acetate or histidine.

In one embodiment of the present invention, the composition may comprise: (A) 90 to 180 mg/ml of an antibody or an antigen-binding fragment thereof, which comprises a light-chain variable region comprising a CDR1 domain comprising an amino acid sequence of SEQ ID NO: 1, a CDR2 domain comprising an amino acid sequence of SEQ ID NO: 2, and a CDR3 domain comprising an amino acid sequence of SEQ ID NO: 3; and a heavy-chain variable region comprising a CDR1 domain comprising an amino acid sequence of SEQ ID NO: 4, a CDR2 domain comprising an amino acid sequence of SEQ ID NO: 5, and a CDR3 domain comprising an amino acid sequence of SEQ ID NO: 6; (B) 0.02 to 0.1% (w/v) of a surfactant; (C) 1 to 10% (w/v) of a sugar or derivatives thereof; and (D) 1 to 50 mM of a buffer comprising acetate or histidine.

In one embodiment of the present invention, the composition may contain: (A) 90 to 180 mg/ml of an antibody or an antigen-binding fragment thereof, which comprises a light-chain variable region comprising a CDR1 domain comprising an amino acid sequence of SEQ ID NO: 1, a CDR2 domain comprising an amino acid sequence of SEQ ID NO: 2, and a CDR3 domain comprising an amino acid sequence of SEQ ID NO: 3; and a heavy-chain variable region comprising a CDR1 domain comprising an amino acid sequence of SEQ ID NO: 4, a CDR2 domain comprising an amino acid sequence of SEQ ID NO: 5, and a CDR3 domain comprising an amino acid sequence of SEQ ID NO: 6; (B) 0.02 to 0.1% (w/v) of polysorbate; (C) 1 to 10% (w/v) of sorbitol; and (D) 1 to 50 mM of a buffer comprising acetate.

In one embodiment of the present invention, the composition may be administered subcutaneously.

In one embodiment of the present invention, the composition may not be subjected to a reconstitution step, a dilution step, or both thereof before use.

In one embodiment of the present invention, the stable composition may be filled into a pre-filled syringe before use.

In one embodiment of the present invention, the composition may be included in an auto-injector before use.

Disease Treatable with Anti-TNFα Antibody

In one embodiment of the present invention, the diseases treatable with the anti-TNFα antibody are selected from the group consisting of rheumatoid arthritis, ulcerative colitis, Crohn's disease, plaque psoriasis, psoriatic arthritis, ankylosing spondylitis, juvenile idiopathic arthritis, hemolytic disease of the newborn, inflammatory bowel disease, multiple sclerosis, prevention of organ transplantation rejection, non-Hodgkin's lymphoma, metastatic cancer, retinopathy of prematurity, ovarian cancer, stomach cancer, head and neck cancer, osteoporosis, paroxysmal nocturnal hemoglobinuria, invasive candidiasis, breast cancer, melanoma, chronic lymphocytic leukemia, acute myeloid leukemia, renal cell carcinoma, colorectal cancer, asthma, nasopharyngeal cancer, hemorrhagic shock, Staphylococcus aureus infection, and follicular lymphoma.

In one embodiment of the present invention, the diseases treatable with the anti-TNFα antibody may be a disease treatable by intravenous administration of infliximab.

In one embodiment of the present invention, the diseases treatable with the anti-TNFα antibody may be rheumatoid arthritis, ulcerative colitis, Crohn's disease, plaque psoriasis, psoriatic arthritis or ankylosing spondylitis, which are treatable by intravenous administration of infliximab.

In one embodiment of the present invention, the subject to be dosed with the anti-TNFα antibody is a patient who has an inadequate response to disease-modifying anti-rheumatic drugs (DMARDs) comprising methotrexate.

In one embodiment of the present invention, the subject to be dosed with the anti-TNFα antibody is a patient who has not previously been treated with methotrexate and other DMARDs.

In one embodiment of the present invention, the subject to be dosed with the anti-TNFα antibody is a patient who exhibits a rise in serologic indicators associated with severe axial symptoms and inflammation, which show no proper response to common therapies.

In one embodiment of the present invention, the subject to be dosed with the anti-TNFα antibody is a patient who does not respond to, is contraindicated from, or has intolerance to methotrexate, cyclosporine, or systemic therapies including dermatologic photochemotherapy (psoralen ultraviolet A therapy: PUVA); In one embodiment of the present invention, the subject to be dosed with the anti-TNFα antibody is a patient who has no adequate response to treatment with corticosteroids, 6-mercaptopurine, azathioprine or immunosuppressants, or has intolerance to such therapy or is contraindicated from such treatment method.

In one embodiment of the present invention, the subject to be dosed with the anti-TNFα antibody is a patient who does not respond to common therapies, comprising antibiotic, excretion or immunosuppressive therapies.

In one embodiment of the present invention, the patient after subcutaneous administration may exhibit one or more characteristics selected from the followings:

a) a decrease in DAS28 (Disease Activity Score in 28 joints) by at least 2.0; or

b) a decrease in CDAI (Crohn's disease activity index) by at least 70.

Administered Dose and Dosing Interval

In one embodiment of the present invention, the anti-TNFα antibody or the antigen-binding fragment thereof may be administered at a dose of 60 to 300 mg. Specifically, it may be administered at a dose of 60, 70, 80, 90, 100, 110, 120, 130, 140, 150, 160, 170, 180, 190, 200, 210, 220, 230, 240, 250, 260, 270, 280, 290 or 300 mg.

In another embodiment of the present invention, the anti-TNFα antibody or the antigen-binding fragment thereof may be administered at a dose of 90 to 180 mg. In another embodiment of the present invention, the anti-TNFα antibody or the antigen-binding fragment thereof may be administered at a dose of 90 to 300 mg. In another embodiment of the present invention, the anti-TNFα antibody or the antigen-binding fragment thereof may be administered at a dose of 120 to 240 mg.

In one embodiment of the present invention, the anti-TNFα antibody or the antigen-binding fragment thereof may be administered at a dose of 80 to 100 mg, 110 to 130 mg, 170 to 190 mg, or 230 to 250 mg.

In one embodiment of the present invention, the anti-TNFα antibody or the antigen-binding fragment thereof may be administered to a patient with rheumatoid arthritis at a dose of 80 to 190 mg, 90 to 180 mg, 110 to 130 mg, 90, 120 or 180 mg.

In one embodiment of the present invention, the anti-TNFα antibody or the antigen-binding fragment thereof may be administered to a patient with ulcerative colitis, Crohn's disease, plaque psoriasis, psoriatic arthritis or ankylosing spondylitis at a dose of 80 to 250 mg, 110 to 250 mg, 110 to 130 mg, 120 to 240 mg, 140 to 160 mg, 170 to 190 mg, 230 to 250 mg, 120, 150, 180 or 240 mg.

In one embodiment of the present invention, the anti-TNFα antibody or the antigen-binding fragment thereof may be administered at a dose of 90 to 180 mg, if the patient's body weight is less than 80 kg, and may be administered at a dose of 190 to 270 mg, if the patient's body weight is 80 kg or more.

In one embodiment of the present invention, the anti-TNFα antibody or the antigen-binding fragment thereof may be administered at an increased dose, if the patient's condition is not improved or therapeutic response is lost. More specifically, the administered dose may be increased 1.1 to 3 times, 1.1 to 2.5 times, 1.1 to 2.1 times, 1.5 to 2.1 times, 1.7 to 2.1 times, and 2 times.

For Crohn's disease, criteria for determining that the therapeutic response is lost may be based on the case in which that the patient's CDAI scores are increased 70 points or more and total scores thereof are 220 points or more. For ulcerative colitis, such criteria may be based on the case in which the patient satisfies the following condition a) and satisfies at least one of b) or c):

a) a rectal bleeding subscore is increased 1 point or more from the minimum score in which an actual value is more than 1 point; and

b) a partial Mayo score is increased two points or more from the minimum score in which an actual value is 4 points or more; or

c) an endoscopic subscore is increased 1 point or more from the minimum score in which an actual value is more than 1 point.

In one embodiment of the present invention, it may be preferable not to further increase a dose, if the patient's condition is not improved and thus a dose of the anti-TNFα antibody or the antigen-binding fragment thereof is increased up to 240 mg. If the patient having received a dose of 240 mg is given an increase from such dose, there may occur a liver damage, etc., caused by a high concentration of drug.

In one embodiment of the present invention, the anti-TNFα antibody or the antigen-binding fragment thereof may be administered at an increased dose from Weeks 5, 10, 15, 20, 21, 22, 23, 24, 25, 26, 27, 28, 29, 30, 31, 32, 33, 32 and 35 or later. More preferably, such increase in dose may be performed from Week 30 or later. If such increase in dose occurs before Week 30, there may be no enough time to identify a medicinal effect of an existing dose. If such increase in dose occurs after Week 30, there may occur a side effect in which the patient' condition is deteriorated.

In one embodiment of the present invention, the anti-TNFα antibody or the antigen-binding fragment thereof may be administered at intervals of 1 to 8 weeks. Specifically, it may be administered at intervals of 1, 1.5, 2, 2.5, 3, 3.5, 4, 4.5, 5, 5.5, 6, 6.5, 7, 7.5, or 8 weeks.

In another embodiment of the present invention, the anti-TNFα antibody or the antigen-binding fragment thereof may be administered at intervals of 2 to 4 weeks.

In one embodiment of the present invention, there may be provided an administration method in which a minimum serum concentration (C_{trough}; minimum concentration immediately before the next application) of the anti-TNFα antibody or the antigen-binding fragment thereof is maintained at 0.01 μg/ml or more after subcutaneous administration to the patient. More specifically, there may be provided an administration method in which the minimum serum concentration is maintained at 0.01 to 50 μg/ml, 0.01 to 45 μg/ml, 0.01 to 40 μg/ml, 0.01 to 35 μg/ml, 0.01 to 30 μg/ml, 0.01 to 25 μg/ml, 0.01 to 20 μg/ml, 0.01 to 15 μg/ml, 0.01 to 10 μg/ml, 0.01 to 6 μg/ml, 0.1 to 6 μg/ml, 5 or 1 μg/ml.

In one embodiment of the present invention, there may be provided an administration method in which the minimum serum concentration (C_{trough}) of the anti-TNFα antibody or the antigen-binding fragment thereof is maintained at 0.01 μg/ml or more, 0.01 to 50 μg/ml, 0.01 to 40 μg/ml, 0.01 to 30 μg/ml, 1 to 40 μg/ml, or 1 μg/ml or more after subcutaneous administration to the patient who has a rheumatoid arthritis disease. Preferably, the minimum serum concentration (C_{trough}) of the anti-TNFα antibody or the antigen-binding fragment thereof may be 1 μg/ml for the patient with rheumatoid arthritis.

In one embodiment of the present invention, there may be provided an administration method in which the minimum serum concentration (C_{trough}) of the anti-TNFα antibody or the antigen-binding fragment thereof is maintained at 0.01 μg/ml or more, 0.01 to 60 μg/ml, 0.01 to 50 μg/ml, 0.01 to 45 μg/ml, 5 to 50 μg/ml, or 5 μg/ml or more after subcutaneous administration to the patient who has one or more diseases selected from the group consisting of ulcerative colitis, Crohn's disease, plaque psoriasis, psoriatic arthritis and ankylosing spondylitis.

Preferably, the minimum serum concentration (C_{trough}) of the anti-TNFα antibody or the antigen-binding fragment thereof may be 5 μg/ml for the IBD patient.

Pre-Administration

Before a step of subcutaneously administering the anti-TNFα antibody or the antigen-binding fragment thereof, the step of intravenously administering the anti-TNFα antibody or the antigen-binding fragment thereof may be comprised.

In one embodiment of the present invention, before a step of subcutaneous administration, the step of intravenously administering the anti-TNFα antibody or the antigen-binding fragment thereof may be performed at least once, and may be performed twice or three times.

In one embodiment of the present invention, a) the patient who has a rheumatoid arthritis disease may be a patient who has been intravenously administered with the anti-TNFα antibody or the antigen-binding fragment thereof twice prior to subcutaneous administration, and b) the patient who has one or more diseases selected from the group consisting of ulcerative colitis, Crohn's disease, plaque psoriasis, psoriatic arthritis and ankylosing spondylitis may be a patient who has been intravenously administered with the anti-TNFα antibody or the antigen-binding fragment thereof twice or three times prior to subcutaneous administration.

In one embodiment of the present invention, the patient may be a patient who has been intravenously administered with the anti-TNFα antibody or the antigen-binding fragment thereof twice at Weeks 0 and 2, or who has been intravenously administered therewith three times at Weeks 0, 2 and 6 prior to subcutaneous administration.

In one embodiment of the present invention, a) the patient who has a rheumatoid arthritis disease may be a patient who has been intravenously administered with the anti-TNFα antibody or the antigen-binding fragment thereof twice at Weeks 0 and 2 prior to subcutaneous administration, and b) the patient who has one or more diseases selected from the group consisting of ulcerative colitis, Crohn's disease, plaque psoriasis, psoriatic arthritis and ankylosing spondylitis may be a patient who has been intravenously administered with the anti-TNFα antibody or the antigen-binding fragment thereof twice at Weeks 0 and 2, or who has been intravenously administered therewith three times at Weeks 0, 2 and 6 prior to subcutaneous administration.

In one embodiment of the present invention, a step of intravenously administering the anti-TNFα antibody or the antigen-binding fragment thereof at a dose of 1 to 10 mg/kg may be comprised before the step of subcutaneous administration. Specifically, the step of intravenously administering at a dose of 1, 2, 3, 4, 5, 6, 7, 8, 9 or 10 mg/kg may be comprised prior thereto.

In another embodiment of the present invention, a step of intravenously administering the anti-TNFα antibody or the antigen-binding fragment thereof at a dose of 2 to 8 mg/kg may be comprised before the step of subcutaneous administration. In another embodiment of the present invention, a step of intravenously administering the anti-TNFα antibody or the antigen-binding fragment thereof at a dose of 3 to 5 mg/kg may be comprised before the step of subcutaneous administration.

In one embodiment of the present invention, a) the patient who has a rheumatoid arthritis disease may be a patient who has been intravenously administered with the anti-TNFα antibody or the antigen-binding fragment thereof at a dose of 3 mg/kg per administration, and b) the patient who has one or more diseases selected from the group consisting of ulcerative colitis, Crohn's disease, plaque psoriasis, psoriatic arthritis and ankylosing spondylitis may be a patient who has been intravenously administered with the anti-TNFα antibody or the antigen-binding fragment thereof at a dose of 5 mg/kg per administration.

In one embodiment of the present invention, a step of intravenously administering the anti-TNFα antibody or the antigen-binding fragment thereof may be comprised before the step of subcutaneous administration, and a step in which an interval between a final intravenous administration and a first subcutaneous administration is one to eight weeks may be comprised therebefore. Specifically, the step of administering at intervals of 1, 1.5, 2, 2.5, 3, 3.5, 4, 4.5, 5, 5.5, 6, 6.5, 7, 7.5 or 8 weeks may be comprised.

In another embodiment of the present invention, a step of intravenously administering the anti-TNFα antibody or the antigen-binding fragment thereof may be comprised before the step of subcutaneous administration, and a step in which an interval between a final intravenous administration and a first subcutaneous administration is two to eight weeks, two to four weeks, or four weeks may be comprised therebefore.

In one embodiment of the present invention, a step of intravenously administering the anti-TNFα antibody or the antigen-binding fragment thereof may be comprised before the step of subcutaneous administration, in which a time interval between a final intravenous administration and a first subcutaneous administration may be one to eight weeks. Specifically, the step of administering at intervals of 1, 1.5, 2, 2.5, 3, 3.5, 4, 4.5, 5, 5.5, 6, 6.5, 7, 7.5 or 8 weeks may be comprised.

In another embodiment of the present invention, a step of intravenously administering the anti-TNFα antibody or the antigen-binding fragment thereof may be comprised before the step of subcutaneous administration, in which a time interval between a final intravenous administration and a first subcutaneous administration may be two to four weeks.

In another embodiment of the present invention, a time of the first subcutaneous administration is set to minimize a possible occurrence of ADA, which may be caused due to a low concentration of infliximab in blood, in such a way that a level of pre-dose concentration comes closest to a blood concentration at a steady state during a period of subcutaneous administration. Considering the conditions above, an optimal interval between the final intravenous administration and the first subcutaneous administration was determined through a simulation which was performed based on a population PK model developed. As a result of the simulation, in two to four weeks after the final intravenous administration, more preferably in four weeks thereafter, i.e., at Week 10, an average blood concentration was most similar to an expected level of pre-dose concentration at steady state within a maintenance phase of subcutaneous administration, and also showed a low change in blood concentration. Thus, by setting the time of first subcutaneous administration at Week 10, it is expected to fastest reach an average level of pre-dose concentration at steady state, which is expected during a test period.

If the first subcutaneous administration is performed within two weeks after the final intravenous administration, a blood concentration may be higher than an expected level of pre-dose concentration at steady state within a maintenance phase of subcutaneous administration. If the first subcutaneous administration is performed at a time point of six weeks after the final intravenous administration, a blood concentration may be lower than an expected level of pre-dose concentration at steady state within a maintenance phase of subcutaneous administration, and the blood concentration may relatively slowly reach an average level of pre-dose concentration at steady state, which is expected during the test period, compared to the case where the subcutaneous administration is performed at a time point of four weeks (at Week 10). In another embodiment of the present invention, the patient may be a patient who has been intravenously administered with the anti-TNFα antibody or the antigen-binding fragment thereof twice at Weeks 0 and 2, or who has been intravenously administered therewith three times at Weeks 0, 2 and 6 prior to subcutaneous administration.

Co-Administration

Other biological agents or chemotherapeutic agents may be administered together with the anti-TNFα antibody or the antigen-binding fragment thereof according to the present invention.

The administration is performed simultaneously with, before or after administration of the anti-TNFα antibody or the antigen-binding fragment thereof.

In one embodiment of the present invention, the biological agents that are co-administered may comprise etanercept, infliximab, adalimumab, certolizumab pegol, golimumab, or a combination thereof.

In one embodiment of the present invention, the chemotherapeutic agents that are co-administered may comprise disease-modifying anti-rheumatic drugs (DMARDs), steroids or immunosuppressants.

In one embodiment of the present invention, the disease-modifying anti-rheumatic drugs (DMARDs) that are co-administered may comprise methotrexate, leflunomide, sulfasalazine, hydroxychloroquine, or a combination thereof.

In one embodiment of the present invention, the steroids that are co-administered may comprise corticosteroid, glucocorticoid, cortisol, mineralocorticoid, aldosterone, or a combination thereof.

In one embodiment of the present invention, the immunosuppressants that are co-administered may comprise azathioprine, 6-mercaptopurine, cyclosporin A, tacrolimus, mycophenolic acid, bredinin, mTOR inhibitor, anti-lymphocyte antibody, or a combination thereof.

Product

The present invention also provides a product comprising: a composition containing an anti-TNFα antibody or an antigen-binding fragment thereof; and a container receiving the composition in a closed state.

The composition containing the anti-TNFα antibody or the antigen-binding fragment thereof is as described above.

In one embodiment of the present invention, the container may be formed of a material such as glass, a polymer (plastic), a metal, etc., but is not limited thereto. In one embodiment of the present invention, the container may be a bottle, a vial, a cartridge, a syringe (pre-filled syringe or auto-injector), or a tube, but is not limited thereto. In one embodiment of the present invention, the container may be a glass or polymer vial, or a glass or polymer pre-filled syringe.

Specific product forms of the above vial, cartridge, pre-filled syringe, auto-injector, etc., and methods for filling the stable liquid pharmaceutical formulation into the vial, cartridge, pre-filled syringe, auto-injector, etc., may be readily available or implemented by those skilled in the art to which the present invention pertains. For example, U.S. Pat. Nos. 4,861,335, 6,331,174, etc. disclose the specific product forms of a pre-filled syringe and filling methods thereof. For example, U.S. Pat. Nos. 5,085,642, 5,681,291, etc. disclose the specific product forms of an auto-injector and assembly methods thereof. The above vial, cartridge, pre-filled syringe, auto-injector, etc., which are used in the present invention may be a commercially available product as it is, or a product separately manufactured considering the physical properties of the composition containing the anti-TNFα antibody or the antigen-binding fragment thereof, an area to be administered, an administered dose thereof, etc.

In one embodiment of the present invention, the inside of the container may not be coated with silicone oil. If it is coated with silicone oil, the stability thereof may be deteriorated. The container may be a single-dose or multiple-dose container.

In one embodiment of the present invention, the product may further comprise instructions providing a method for using the composition containing the anti-TNFα antibody or the antigen-binding fragment thereof, a method for storing the composition, or both thereof. The method for using the composition may comprise a method for treating diseases in which TNFα activity is harmful, and may comprise a route of administration, an administered dose and a timing of administration.

In one embodiment of the present invention, the product may comprise other required tools, for example, a needle, a syringe, etc. in a commercial viewpoint and a user's viewpoint.

Hereinafter, the present invention will be described with reference to examples. It is to be understood, however, that these examples are for illustrative purposes only and are not intended to limit the scope of the present invention.

### Example 1. Evaluation of Safety and Therapeutic Efficacy on Subcutaneous Administration of Infliximab to Patients with Crohn's Disease (CD) or Ulcerative Colitis (UC) (Study 1.6)

### Example 1-1. Study Protocol

The present study of infliximab (CT-P13) was an open, randomized, multi-center, parallel-group and phase I trial, designed to evaluate the pharmacokinetics, efficacy and safety between infliximab for subcutaneous administration (hereinafter infliximab SC) and infliximab for intravenous administration (hereinafter infliximab IV) in patients with active CD or active UC until 54 weeks, in which the present study was composed of two parts.

Part 1 was designed to identify an optimal dose of the infliximab SC in CD patients, in which the optimal dose of the infliximab SC corresponding to 5 mg/kg of the infliximab IV over the first 30 weeks was identified by means of an area under the concentration-time curve (AUC_{τ}) at steady state between Weeks 22 and 30. In case of Part 1, a study period lasted for a maximum of 65 weeks, including a duration from Screening (the maximum of three weeks) to End-of-Study visit.

Part 2 was designed to identify that the infliximab SC is not pharmacokinetically inferior to the infliximab IV in CD or UC patients, which was demonstrated by a means of pre-dose serum concentration (C_{trough}) at Week 22. In Part 2, an optimal administered dose and dosing interval of the infliximab SC corresponding to 5 mg/kg of the infliximab IV was determined as follows, by an independent Data Safety Monitoring Board (DSMB) based on the pharmacokinetics, efficacy, pharmacodynamics and safety data over the first 30 weeks of Part 1:
Patients weighing less than 80 kg: Dosed with 120 mg of the infliximab SC every 2 weeks; and
Patients weighing 80 kg or more: Dosed with 240 mg of the infliximab SC every 2 weeks.

Part 1

Patients had to meet all the following inclusion criteria to be enrolled in this study.
Patient who suffered from an active disease with a score on the Crohn's disease activity index (CDAI) of 220-450 points;
Patient who had been diagnosed with CD at least 3 months prior to the first administration of the study drug; and
Patient who had been treated for active CD, but had not responded despite a full and adequate course of therapy with a corticosteroid and/or an immunosuppressant; or who was intolerant to or had medical contraindications for such therapy.

Patients meeting any of the following criteria were excluded from this study.
Patient who had previously received a biological agent for the treatment of CD and/or a TNFα inhibitor for the treatment of other diseases;
Patient who had allergies to any of the excipients of infliximab or any other murine and/or human proteins, or patient who had a hypersensitivity to immunoglobulin products;
Patient who had active entero-vesical, entero-retroperitoneal, entero-cutaneous, and entero-vaginal fistulas within 6 months prior to the first administration of the study drug (Day 0). Entero-enteral fistulas without any clinically significant symptoms according to an investigator's opinion and anal fistulas without draining problems were allowed;
Patient who had taken more than three small-bowel resection procedures prior to the first administration of the study drug (Day 0);
Patient who had a current or past history of chronic infection with hepatitis C, human immunodeficiency virus (HIV-1 or HIV-2), or current infection with hepatitis B; and
Female patient who was pregnant.

The study was comprised 3 study periods including Screening, Treatment Period (Dose-Loading Phase and Maintenance Phase) and the End-of-Study. The screening took place between Days −21 and −1, prior to the first administration of the study drug, in which the eligibility of patients for study was determined. All the assessments including hepatitis B, hepatitis C and human immunodeficiency virus (HIV-1 and HIV-2) infections status, a urine and serum pregnancy test for women of childbearing potential, colonoscopy, CRP, 12-lead ECG, clinical laboratory tests, etc., were performed. Also, an interferon-gamma release assay (IGRA) and a chest X-ray examination were performed so as to exclude patients with active tuberculosis (TB).

On Day 0, Week 0, the patients who met all inclusion criteria and none of the exclusion criteria were enrolled in the study, and infliximab IV were given to all enrolled patients at Weeks 0 and 2. The patients could be premedicated 30 to 60 minutes prior to the start of study drug administration and any premedications such as but not limited to antihistamine (at equivalent dose of 2-4 mg of chlorpheniramine), hydrocortisone, paracetamol, and/or nonsedating antihistamine (at equivalent dose of 10 mg of cetirizine) could be given at the investigator's discretion.

Patients who received 2 full doses and for whom there were no safety concerns based on the investigator's discretion were randomly assigned to receive either infliximab SC or infliximab IV before treatment on Day 42, Week 6. The randomization to treatment assignment was stratified by region (Europe or non-Europe), a current use of azathioprine or 6-mercaptopurine or MTX treatment (used or not used), a presence of clinical responses by means of CDAI-70 at Week 6 and a weight (70 kg or less, or more than 70 kg) at Week 6. A total of 45 patients with active CD were enrolled, out of which 44 patients were randomly assigned at Week 6 in a 1:1:1:1 ratio into 4 study cohorts, in which the study drug was administered up to Week 54 (Table 1).

TABLE 1

| | | | | Dosage, Investigational | |
|---|---|---|---|---|---|
| Cohort | | | | Product, Metbod of | Number of |
| Number | Dosage | | Investigational Product | Administration | Patients |
| Cohort 1 | 5 | mg/kg | infliximab IV 100 mg/vial | Two hours IV infusion | 13 |
| Cohort 2 | 120 | mg | infliximab SC 120 mg/PFS* | Single SC injection | 11 |
| Cohort 3 | 180 | mg | infliximab SC 90 mg/PFS | Double SC injections | 12 |
| Cohort 4 | 240 | mg | infliximab SC 120 mg/PFS | Double SC injections | 8 |

*PFS, Pre-filled syringe

Those who were assigned to Cohort 1 received additional 7 doses of the infliximab IV at Week 6 and subsequently every 8 weeks (Weeks 14, 22, 30, 38, 46 and 54). Those who were assigned to Cohorts 2, 3 and 4 were initially dosed with the infliximab SC at Week 6, and then additionally dosed with the infliximab SC every other week up to Week 54. Dose escalation up to 10 mg/kg was allowed for patients in Cohort 1 since Week 30 if the patient initially responded but then lost response at each visit. The initial dose assigned to all the patients of Cohorts 2, 3 and 4 was adjusted to an optimal dose after the optimal dose was confirmed through dose finding. After that, an additional SC injection using the optimal dose was performed until Week 54. The infliximab SC was injected into patients by a healthcare provider at each study center (Weeks 6, 8, 10, 14, 22, 24, 26, 28, 30, 38, 46 and 54). After proper training in injection technique, patients could self-inject with infliximab SC in all the other weeks (Weeks 12, 16, 18, 20, 32, 34, 36, 40, 42, 44, 48, 50 and 52), if the investigator determined that it was appropriate. Patients returned to the study center at predefined time intervals for clinical assessments and blood sampling. At each visit, the patients were asked questions about adverse events (AEs) and concomitant medications, while being monitored for clinical signs and symptoms of tuberculosis (TB). The evaluation of primary pharmacokinetic endpoint was performed at steady state between Weeks 22 and 30, then the evaluation of secondary pharmacokinetic endpoints was performed during the treatment until Week 54, and then blood sampling for analysis as well as the evaluation of efficacy, PD and safety were respectively performed at a time points specified in an schedule of events.

An End-of-Study visit was occurred 8 weeks after the end of a maintenance phase. However, the visit was performed 8 weeks after the last dose was received, if the patients withdrew from the study. For patients who dropped out for any reason, all study procedures were performed on the day of withdrawal (or the day after withdrawal) and all attempts were made to complete all End-of-Study assessments at the time points of 8 weeks after the last dose was received.

Part 2

Part 2 initiated based on a review by the independent Data Safety Monitoring Board (DSMB) with regard to PK modeling report data including PK, efficacy, PD and safety data, which were identified over the first 30 weeks in Part 1.

Patients had to meet all the following criteria to be enrolled in this study.

Active Crohn's Disease Inclusion Criteria
Patient who suffered from an active disease with a score on the Crohn's disease activity index (CDAI) of 220-450 points;
Patient who had been diagnosed with CD at least three months prior to the first administration of the study drug; and
Patient who had been treated for active CD, but had not responded despite a full and adequate course of therapy with a corticosteroid and/or an immunosuppressant; or who was intolerant to or had medical contraindications for such therapy.
Patient met at least 1 of the following items:
A serum concentration of C-reactive protein (CRP) was more than 0.5 mg/dL;
A concentration of fecal calprotectin was more than 100 μg/g; and
Simplified Endoscopic Activity Score for CD (SES-CD) of ≥6 points for ileal-colonic CD or ≥4 points including ulcer score from at least 1 segment for ileal CD or colonic CD.

Active Ulcerative Colitis Inclusion Criteria
Patient who had active UC as defined by a total Mayo score between 6 and 12 points with endoscopic evidence of active colitis as indicated by endoscopic subscore of ≥2 at screening.
Patient who had UC of at least 3 months' disease duration prior to the first administration of the study drug (Day 0).
Patient who had been treated for active UC but not responded despite conventional therapy including corticosteroids alone or in combination with 6-MP or AZA and medications containing 5-ASA, or who was intolerant to or had medical contraindications for such therapies.

Patients meeting any of the following criteria were excluded from the Part 2 of the study.
Patient who had previously received a biological agent for the treatment of CD or UC and/or a TNFα inhibitor for the treatment of other diseases;
Patient who had allergies to any of the excipients of infliximab or any other murine and/or human proteins, or patient who had a hypersensitivity to immunoglobulin products;
CD patient who had active entero-vesical, entero-retroperitoneal, entero-cutaneous, and entero-vaginal fistulas within 6 months prior to the first administration of the study drug (Day 0). Entero-enteral fistulas without any clinically significant symptoms according to an investigator's opinion and anal fistulas without draining problems were allowed;
CD patient who had taken more than 3 small-bowel resection procedures prior to the first administration of the study drug (Day 0);
UC patient who had been rectally administered medications containing corticosteroids or 5-aminosalicylic acid for the treatment of UC 2 weeks prior to Screening;
Patients who had more than 8 years of disease ducation of UC must have had documented evidence for absence of colorectal cancer or dysplasia by full colonoscopy examination performed within one year prior to the first administration of the study drug (Day 0);
Patient who was infected with hepatitis B, hepatitis C and human immunodeficiency virus (HIV-1 and HIV-2); and
Patient who was pregnant.

Part 2 was composed of three study periods: Screening, Treatment Period, and End-of-Study. The Screening was carried out between Days −42 and 0 before the first administration of the study drug, in which the eligibility of patients for study was evaluated. All the examinations including hepatitis B, hepatitis C and human immunodeficiency virus (HIV-1 and HIV-2) infections, a urine and serum pregnancy test for women of childbearing potential, colonoscopy (patients with CD), proctosigmoidoscopy (patients with UC), CRP, 12-lead ECG, clinical laboratory tests, etc., were carried out. Also, an interferon-gamma release assay (IGRA) and a chest X-ray examination were performed so as to exclude tuberculosis (TB) patients.

Only the patients who met all the inclusion criteria and none of the exclusion criteria at Day 0 and Week 0 were enrolled into the study, and all the patients enrolled were dosed with the infliximab IV twice at Weeks 0 and 2 at a single dose, respectively. The patients were eligible to take the following premedication (without a limitation thereto) at the investigator's discretion at a time point of 30-60 minutes before a start of the administration of the study drug so that their hypersensitivity reactions to the study drug might be prevented (e.g., antihistamine [at an equivalent dose of 2-4 mg of chlorpheniramine], hydrocortisone, paracetamol and/or non-sedating antihistamine [at an equivalent dose of 10 mg of cetirizine]).

Those who received a full dose of the study drug twice and deemed to have no concerns about safety at the investigator's discretion, were randomly assigned to a treatment arm dosed with the infliximab SC or a treatment arm dosed with the infliximab IV before the administration on Day 42 and Week 6. Such randomization with regard to administration of the study drug was stratified by a current use of azathioprine or 6-mercaptopurine or MTX treatment (used or not used), a presence of diseases (CD or UC), a presence of CD clinical responses by means of CDAI-70 or UC clinical responses by means of partial Mayo score at Week 6 and a bodyweight (less than 80 kg, or 80 kg or more) at Week 6. A total of 131 patients with active CD or active UC were randomly assigned at Week 6 to two study treatment arms at a ratio of 1:1, in which the administration of the study drug was performed until Week 54 (Table 2).

TABLE 2

| Treatment | Administered | Arm number, Dosage, | Administration | Number of |
|---|---|---|---|---|
| Arm Number | Dose | Investigational Product | Method | Patients |
| Treatment Arm 1 | 5 mg/kg | infliximab IV 100 mg/vial | Two hours IV infusion | 65 |
| Treatment Arm 2 | 120 mg (<80 kg) | infliximab SC 120 mg/PFS* | Single SC injection | 66 |
| | 240 mg (≥80 kg) | infliximab SC 120 mg/PFS | Double SC injections | |

*PFS, Pre-filled syringe

Those who were assigned to Treatment Arm 1 further received the infliximab IV at Week 6 and subsequently every 8 weeks (Weeks 14 and 22) until Week 22, and then such the IV 5 mg/kg was switched to the SC 120/240 mg at Week 30, in which an SC dose was determined based on a body weight at Week 30. After that, the infliximab SC was administered at the dose above every 2 weeks until Week 54. In case of those who were assigned to Treatment Arm 2, a dose of the infliximab SC was determined based on their body weights at Week 6, and the infliximab SC was administered at such dose every 2 weeks from Week 6 to 54. An increase in dose was permitted from Week 30 according to the investigator's decision. The infliximab SC was injected into patients by a healthcare provider at each site visit (Weeks 6, 14, 22, 24, 26, 28, 30, 38, 46 and 54). However, in all the other weeks, patients were allowed to perform a self-injection of the infliximab SC, if the investigator determined it as appropriate after training the patients for proper injection techniques.

The evaluation of primary pharmacokinetic endpoint was performed at Week 22, and then the evaluation of secondary pharmacokinetic endpoints was performed during a steady state between Weeks 22 and 30 and during the treatment period until Week 54. Blood sampling for analysis as well as the evaluation of efficacy, PD and safety were performed at a point of time specified in a schedule of events.

An End-of-Study visit occurred 2 weeks after the end of a maintenance phase. However, such visit occurred 2 weeks after the last time point of administration, if patients discontinued the study halfway after the SC administration. However, such visit occurred 8 weeks after the last time point of administration, if patients discontinued the study halfway after the IV administration. In case of dropout patients, all study procedures were performed on a day of dropout or on the next day after such dropout, in which every effort was made to complete all the End-of-Study assessments at a pre-determined time point after the last administration to patients.

In case of Part 2, clinical evaluation, blood sampling and study visits for each type were performed in the same way as shown in Part 1 as well as at a point of time specified in a schedule of events.

### Example 1-2. Efficacy Evaluation Through PK-PD Modeling

Development of PK-PD Model

A population pharmacokinetic-pharmacodynamic (PK-PD) model for the CT-P13 subcutaneous (SC) administration was established not only to simulate the PK of future administered doses and regimens, but also to simulate the efficacy of the CT-P13 SC. The population PK-PD model was based on the CT-P13 IV administration data on healthy volunteers (HV), patients with ankylosing spondylitis (AS), patients with rheumatoid arthritis (RA) and patients with Crohn's disease (CD), as well as the infliximab SC administration data on patients with CD, patients with UC, patients with RA and patients with HVs (Clinicaltrials.gov Identifier Code NCT01220518 (Study 1.1), NCT01217086 (Study 3.1), NCT02096861 (Study 3.4) and NCT02883452 (Study 1.6)).

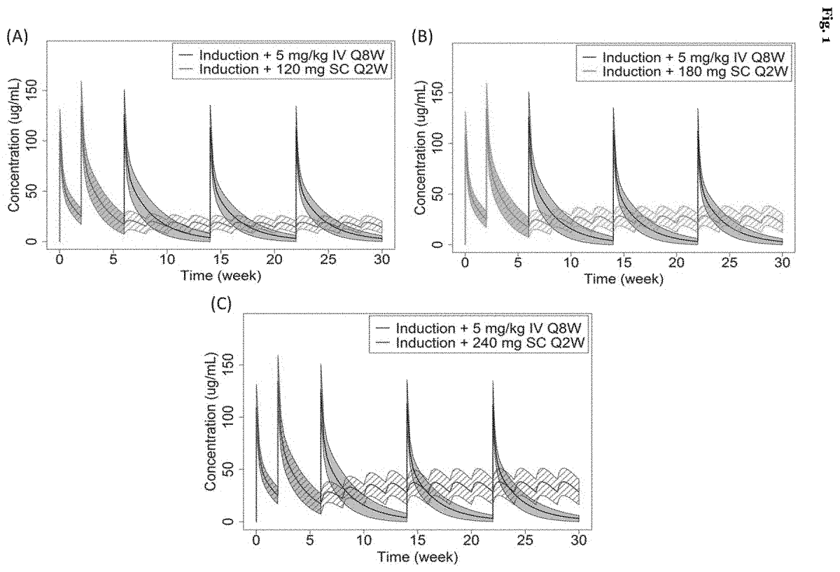

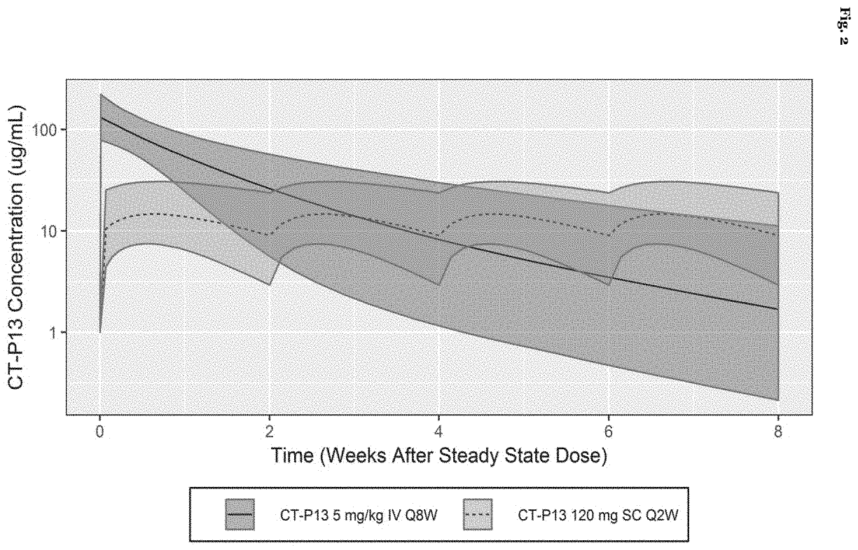

The PK-PD model developed based on the data above was used to simulate the SC administration results for patients having the indications of infliximab (RA, CD or UC). As a result of the PK-PD modeling of Study 1.6 Part 1, FIG. 1 was aimed at finding optimal dose of the infliximab SC injection in CD patients of Part 1. An optimal dose of Study 1.6 Part 2 was determined based on the results of the PK-PD modeling of Study 1.6 Part 1 and the results of pharmacokinetics, efficacy and safety of Study 1.6 Part 1. FIG. 2 was delivered as a result of the PK-PD modeling of Study 1.6 Part 2 with an addition of the results of Study 1.6 Part 2 until Week 30. It was identified if an optimal dose of the infliximab SC for patients with inflammatory bowel disease (IBD) achieves target therapeutic serum concentration (5 μg/ml) through the PK-PD modeling of Study 1.6 Part 2. The target therapeutic serum concentration for IBD patients was determined through a comprehensive literature search.

The PK-PD modeling analysis was performed by a nonlinear mixed effect modeling approach. The starting point for the population PK analysis of infliximab was a one compartment infusion model with linear elimination with a proportional error model, and then a final model was performed by a 2-compartment model having a linear elimination from a central compartment. All models were parameterized in terms of clearance (CL) and volume (V).

As for the final PK model of Part 1 and Part 2, a profile was predicted in such a way that each predicted value for parameters such as an area under the concentration-time curve (AUC_{τ}) and a minimum serum concentration (C_{trough}: minimum concentration immediately before the next application) in the CT-P13 study was applied to each actual dose, regimen and administration route. Also, an additional simulation was performed so as to evaluate an effect on a fixed dose, regimen and administration route for each weight. The PK-PD modeling and simulation of subcutaneous doses in Study 1.6 Part 1 were performed by means of NONMEM v7.2, and the PK-PD modeling and simulation of Part 2 were performed by means of NONMEM v7.3.0.

As shown in FIG. 1 and Tables 3 and 4 (PK-PD modeling of Study 1.6 Part 1), it was demonstrated that a valid target therapeutic serum concentration (5 ug/ml) achieved in infliximab SC who administered with 120, 180, and 240 mg from Week 6 compared to a control cohort (Induction+5 mg/kg IV Q8W). Further, it was demonstrated that each PK parameter value is increased in a dose proportional manner at steady state. It was also demonstrated that C_{trough} was higher and Cₘₐₓ was lower in infliximab SC than in IV control cohort. It was demonstrated that this trend occurs as the drug is slowly absorbed from subcutaneous into the systemic circulation due to the characteristics of SC formulation and it was predicted that AUC_{τ} was similar between 120 mg infliximab SC and IV control cohort.

TABLE 3
Summary of infliximab exposure results with median value at steady state (5-95 percentile of prediction
interval) with regard to simulated fixed dose therapy of infliximab (Study 1.6 Part 1)

| Simulated Dose Regimen | C_{trough} (ug/ml) | AUC_{22-30 wk} (h · μg/ml) | Cₘₐₓ (μg/ml) |
|---|---|---|---|
| 5 mg/kg IV administration | 2.3 (0.14-8.57) | 25599.73 (12973-98-41202.48) | 103.69 (78.58-136.63) |
| every 8 weeks | | | |
| 120 mg SC administration | 13.27 (5.62-26.77) | 21923.15 (11227.54-41085-68) | 18.24 (10.14-32.63) |
| every 2 weeks | | | |
| 180 mg SC administration | 19.91 (8.43-40.01) | 32882.88 (16841.3-61505.99) | 27-36 (15-21-48.88) |
| even- 2 weeks | | | |
| 240 mg SC administration | 26.54 (11-23-53-24) | 43842.61 (22455.07-81926.3) | 36.48 (20.28-65.15) |
| every 2 weeks | | | |

TABLE 4
Least Square Mean Ratios of simulated C_{trough} at steady state for infliximab
SC therapy (test cohort) vs. for IV Reference Regimen (control cohort)

| | | Mean of | Mean of | Proportion | Proportion |
|---|---|---|---|---|---|
| | | test | control | of C_{trough} | of 90% |
| | | cohort | cohort | least mean | confidence |
| Comparison | Number | (C_{trough}) | (C_{trough}) | square | interval |
| 120 mg SC administration every 2 | 265 | 12.8 | 1.7 | 7.76 | 6.57:9.16 |
| weeks vs. Control cohort | | | | | |
| 180 mg SC administration every 2 | 265 | 19.2 | 1.7 | 11.62 | 9.85:13.72 |
| weeks vs. Control cohort | | | | | |
| 240 mg SC administration every 2 | 265 | 25.6 | 1.7 | 15.49 | 13.12:18.28 |
| weeks vs. Control cohort | | | | | |

As shown in FIG. 2 (PK-PD modeling of Study 1.6 Part 2), if the infliximab SC 120 mg is administered to CD and UC patients regardless of body weights, it was demonstrated that a serum concentration of infliximab was constantly maintained through the steady state and exceeds the target therapeutic concentration, compared to the cohort administered infliximab IV 5 mg/kg. It was also demonstrated that the C_{trough} and Cₘₐₓ of the 120 mg infliximab SC show the same trend as shown in the results of PK-PD modeling (FIG. 1) in Study 1.6 Part 1. Based on the results of PK-PD modeling in Study 1.6 Part 2, it is predicted that the administration of SC 120 mg every other week would exhibit a valid therapeutic effect on IBD patients regardless of body weights. Consequently, based on the final results of PK-PD modeling, it was confirmed that the administration of SC 120 mg every other week is an optimal dose for IBD patients.

### Example 1-3. Actual Clinical Results (Study 1.6 Part 2)

Safety Evaluation

### Example 1-3-1. Summary of Adverse Events

The safety assessments (secondary endpoints for Part 2) were performed on immunogenicity, hypersensitivity monitoring (including delayed hypersensitivity monitoring), measurement of vital signs (including blood pressure, heart and respiratory rates, and body temperature), weight, interferon-gamma release assay (IGRA), chest X-ray, hepatitis B, hepatitis C and human immunodeficiency virus (HIV1 and HIV-2) infectious states, findings on physical examination, 12-lead ECG, adverse events (including serious adverse events (hereinafter SAEs)), adverse events of special interest (infusion-related reaction/hypersensitivity/anaphylactic reaction (administration-related reaction), delayed hypersensitivity reaction, injection site reaction, infection and malignancies), signs and symptoms of tuberculosis (TB), clinical laboratory analysis, pregnancy test, prior and concomitant medications, and local site pain using a 100 mm visual analogue scale (VAS).

The cumulative safety data of the present study included the adverse events reported until Week 54, in which an overall summary of treatment-emergent adverse events (TEAEs) during a maintenance phase (Weeks 6 to 54) was presented in Table 5. Overall, 363 TEAEs were reported in 87 (66.4%) patients-38 patients (58.5%) from the IV 5 mg/kg treatment arm and 49 patients (74.2%) from the SC 120/240 mg treatment arm respectively, thus indicating a similar proportion between the two arms. The majority of TEAEs were grade 1 or 2 in intensity.

The treatment-emergent serious adverse events (TESAEs) were reported in 11 patients (8.4%)-6 patients (9.2%) from the IV 5 mg/kg treatment arm and 5 patients (7.6%) from the SC 120/240 mg treatment arm, respectively. Out of all the TESAEs, 3 patients (2.3%) were regarded to be related to the study drug by the investigator.

The TEAEs classified as administration-related reaction were occurred in 2 patients (3.1%) from the IV 5 mg/kg treatment arm and in 3 patients (4.5%) from the SC 120/240 mg treatment arm. Out of the TEAEs, the delayed hypersensitivity reaction was occurred in 2 patients (3.0%) in the SC 120/240 mg treatment arm only.

The TEAEs classified as localized injection site reaction were occurred in 3 patients (4.6%) from the IV 5 mg/kg treatment arm and in 15 patients (22.7%) from the SC 120/240 mg treatment arm. A higher proportion of adverse events classified as the injection site reaction were occurred from the SC 120/240 mg treatment arm. A high proportion of adverse events resulted from a difference depending on the administration route, in which the intensity thereof was all shown as the grade 1 or 2 and most of the patients were recovered without a separate treatment.

The TEAEs classified as infection were occurred in 19 patients (29.2%) from the IV 5 mg/kg treatment arm and in 21 patients (31.8%) from the SC 120/240 mg treatment arm.

The TEAEs leading to discontinuation of the study drug were reported in 3 patients (4.6%) from the IV 5 mg/kg treatment arm and in 1 patient (1.5%) from the SC 120/240 mg treatment arm.

TABLE 5

| | Maintenance phase (Weeks 6-54) | |
|---|---|---|
| | IV 5 mg/kg | SC 120/240 mg |
| | (N = 65) | (N = 66) |
| Number of patients with at least | 38 (58.5) | 49 (74.2) |
| one TEAEs (%) | | |
| Related | 20 (30.8) | 28 (42.4) |
| Unrelated | 32 (49.2) | 41 (62.1) |
| Number of patients with at least | 6 (9.2) | 5 (7.6) |
| one TESAEs (%) | | |
| Related | 2 (3.1) | 1 (1.5) |
| Unrelated | 5 (7.7) | 4 (6.1) |
| Number of patients with at least one | 2 (3.1) | 3 (4.5) |
| TEAEs classified as administration- | | |
| related reaction (%) | | |
| Number of patients with at least one | 3 (4.6) | 15 (22.7) |
| TEAEs classified as localized | | |
| injection site reaction (%) | | |
| Number of patients with at least one | 19 (29.2) | 21 (31.8) |
| TEAEs classified as infection (%) | | |
| Number of patients with at least one | 3 (4.6) | 1 (1.5) |
| TEAEs leading to study drug | | |
| discontinuation (%) | | |

At each of summarization, patients were counted once, if they reported more than one event. Only the most severe event was counted. Each event was considered to be related to the study drug by the investigator, only if the relationship is defined as ‘Possible,’ ‘Probable’ or ‘Definite’.

### Example 1-3-2. Immunogenicity Evaluation

As shown in the following Table 6, the proportion of patients with positive ADA in the SC 120/240 mg treatment arm was not higher than that of the IV 5 mg/kg treatment arm until Week 30 and the proportion of patients with positive ADA results in the IV 5 mg/kg treatment arm was not increased even after switching to an SC 120/240 mg treatment arm from Week 30. The proportion of patients with positive ADA results was generally similar between SC and IV treatment arms until Week 54, and the proportion of patients who had ever been identified as positive for the ADA even once after administration of the drug at Week 0 was similar between the treatment arms.

TABLE 6

| | | IV 5 mg/kg | SC 120/240 mg | Total |
|---|---|---|---|---|
| Visit | n (%) | (N = 65) | (N = 66) | (N = 131) |
| Week 0 | ADA positive | 2 (3.1) | 0 | 2 (1.5) |
| | NAb positive | 0 | 0 | 0 |
| Week 6 | ADA positive | 7 (10.8) | 3 (4.5) | 10 (7.6) |
| | NAb positive | 1 (1.5) | 1 (1.5) | 2 (1.5) |
| Week 14 | ADA positive | 19 (29.2) | 14 (21.2) | 33 (25.2) |
| | NAb positive | 9 (13.8) | 7 (10.6) | 16 (12.2) |
| Week 22 | ADA positive | 32 (49.2) | 21 (31.8) | 53 (40.5) |
| | NAb positive | 12 (18.5) | 4 (6.1) | 16 (12.2) |
| Week 30 | ADA positive | 35 (53.8) | 25 (37.9) | 60 (45.8) |
| | NAb positive | 19 (29.2) | 2 (3.0) | 21 (16.0) |
| Week 38 | ADA positive | 27 (41.5) | 29 (43.9) | 56 (42.7) |
| | NAb positive | 10 (15.4) | 4 (6.1) | 14 (10.7) |
| Week 46 | ADA positive | 23 (35.4) | 32 (48.5) | 55 (42.0) |
| | NAb positive | 9 (13.8) | 5 (7.6) | 14 (10.7) |
| Week 54 | ADA positive | 25 (38.5) | 31 (47.0) | 56 (42.7) |
| | NAb positive | 9 (13.8) | 4 (6.1) | 13 (9.9) |

| | | | |
|---|---|---|---|
| At least one ADA positive after | 40/63 (63.5) | 46/66 (69.7) | 86/129 (66.7) |
| administration at Week 0 * | | | |
| At least one NAb positive after | 24/65 (36.9) | 12/66 (18.2) | 36/131 (27.5) |
| administration at Week 0 * | | | |

* Abbreviation: ADA: anti-drug antibody; NAb: neutralizing antibody
* Numerator: Number of patients who had ever been identified as positive for the ADA or the NAb even once after administration of the study drug at Week 0; Denominator: Number of patients who had ever had immunogenicity results even once after administration of the study drug at Week 0 and had never been identified as positive for the ADA or the NAb before administration of the study drug at Week 0; the End-of-Study visit was not counted.

### Example 1-3-3. Local Site Pain Assessment Using the Visual Analogue Scale (VAS)

A range of the visual analogue scale (VAS) was from 0 to 100 mm, with higher scores indicating more severe pains. As shown in the following Table 7, a slightly higher level of VAS was observed in the SC 120/240 mg treatment arm at Week 6, i.e., first time of infliximab SC administration. However, it was identified that such pain tends to decrease until Week 38 as the SC administration is repeated every 2 weeks. In case of the IV 5 mg/kg treatment arm, which was switched to the SC 120 mg at Week 30, a slightly higher level of local site pain was observed at Week 30. However, the local site pain was decreased at Week 38 due to the subsequent SC administration every 2 weeks. The local site pain was slightly increased in both treatment arms at Week 46, and then decreased at Week 54.

TABLE 7

| | | | |
|---|---|---|---|
| Visit | IV 5 mg/kg | SC 120/240 mg | Total |
| Statistics | (N = 65) | (N = 66) | (N = 131) |

Week 6

| | | | |
|---|---|---|---|
| Number | 65 | 66 | 131 |
| Mean (Standard Deviation, SD) | 6.68 (14.897) | 12.48 (17.419) | 9.60 (16.414) |
| Median (Minimum, Maximum) | 1.00 (0.0, 68.0) | 3.25 (0.0, 87.0) | 2.00 (0.0, 87.0) |

Week 14

| | | | |
|---|---|---|---|
| Number | 64 | 63 | 127 |
| Mean (SD) | 7.20 (13.320) | 9.05 (17.130) | 8.12 (15.296) |
| Median (Minimum, Maximum) | 1.25 (0.0, 56.0) | 2.00 (0.0, 79.0) | 2.00 (0.0, 79.0) |

Week 22

| | | | |
|---|---|---|---|
| Number | 57 | 58 | 115 |
| Mean (SD) | 7.06 (15.143) | 10.44 (18.970) | 8.76 (17.189) |
| Median (Minimum, Maximum) | 1.00 (0.0, 68.0) | 2.50 (0.0, 85.0) | 2.00 (0.0, 85.0) |

Week 30

| | | | |
|---|---|---|---|
| Number | 55 | 59 | 114 |
| Mean (SD) | 11.24 (17.497) | 7.68 (13.851) | 9.39 (15.747) |
| Median (Minimum, Maximum) | 3.00 (0.0, 78.0) | 3.00 (0.0, 80.0) | 3.00 (0.0, 80.0) |

Week 38

| | | | |
|---|---|---|---|
| Number | 53 | 59 | 112 |
| Mean (SD) | 6.58 (10.379) | 7.60 (13.104) | 7.12 (11.851) |
| Median (Minimum, Maximum) | 2.00 (0.0, 51.0) | 3.00 (0.0, 60.0) | 3.00 (0.0, 60.0) |

Week 46

| | | | |
|---|---|---|---|
| Number | 51 | 57 | 108 |
| Mean (SD) | 12.29 (22.069) | 13.18 (22.630) | 12.76 (22.267) |
| Median (Minimum, Maximum) | 4.00 (0.0, 95.0) | 3.00 (0.0, 89.0) | 3.75 (0.0, 95.0) |

Week 54

| | | | |
|---|---|---|---|
| Number | 49 | 54 | 103 |
| Mean (SD) | 6.85 (10.722) | 10.34 (20.751) | 8.68 (16.761) |
| Median (Minimum, Maximum) | 2.50 (0.0, 52.0) | 3.00 (0.0, 99.0) | 3.00 (0.0, 99.0) |

### Therapeutic Efficacy Evaluation

### Example 1-3-4. Disease Activity Index Measured by CDAI (Patients with Crohn's Disease)

As shown in the following Table 8, a mean of CD activity index (CDAI) at Week 6 tended to be higher in the SC 120/240 mg treatment arm than in the IV 5 mg/kg treatment arm after initial IV loading regimen consisting of 2 doses of IV 5 mg/kg. However, the CDAI scores were continuously decreased in both treatment arms and thus the mean of the CDAI scores and mean change from baseline of CDAI scores were similar between both treatment arms up to Week 30. The mean change from baseline of CDAI scores was similar between the two treatment arms until Week 54 excluding Week 46 after the IV 5 mg/kg was switched to the SC 120/240 mg at Week 30.

TABLE 8

| | |
|---|---|
| IV 5 mg/kg | SC 120/240 mg |
| (N = 65) | (N = 66) |

| | | | | |
|---|---|---|---|---|
| Visit | Actual | Change from | Actual | Change from |
| Statistics | Result | Baseline | Result | Baseline |

Baseline

| | | |
|---|---|---|
| Number | 25 | 28 |
| Mean | 294.5 | 296.38 |
| SD | 59.899 | 59.212 |

Week 2

| | | | | |
|---|---|---|---|---|
| Number | 24 | 24 | 28 | 28 |
| Mean | 191.03 | −99.66 | 194.89 | −101.49 |
| SD | 79.250 | 71.877 | 74.943 | 83.343 |

Week 6

| | | | | |
|---|---|---|---|---|
| Number | 25 | 25 | 28 | 28 |
| Mean | 144.94 | −149.81 | 164.99 | −131.40 |
| SD | 80.123 | 83.563 | 96.36 | 103.753 |

Week 14

| | | | | |
|---|---|---|---|---|
| Number | 25 | 25 | 27 | 27 |
| Mean | 131.47 | −163.28 | 136.29 | −160.64 |
| SD | 71.444 | 84.169 | 85.918 | 100.957 |

Week 22

| | | | | |
|---|---|---|---|---|
| Number | 21 | 21 | 24 | 24 |
| Mean | 105.08 | −194.43 | 106.55 | −193.00 |
| SD | 60.970 | 74.283 | 80.461 | 89.087 |

Week 30

| | | | | |
|---|---|---|---|---|
| Number | 20 | 20 | 24 | 24 |
| Mean | 106.44 | −187.09 | 103.81 | −195.74 |
| SD | 67.705 | 93.865 | 88.435 | 100.678 |

Week 38

| | | | | |
|---|---|---|---|---|
| Number | 19 | 19 | 24 | 24 |
| Mean | 88.17 | −207.22 | 96.53 | −203.03 |
| SD | 69.015 | 89.79 | 94.341 | 110.301 |

Week 46

| | | | | |
|---|---|---|---|---|
| Number | 18 | 18 | 23 | 23 |
| Mean | 102.32 | −187.69 | 95.01 | −205.45 |
| SD | 83.808 | 101.699 | 107.142 | 129.425 |

Week 54

| | | | | |
|---|---|---|---|---|
| Number | 18 | 18 | 22 | 22 |
| Mean | 79.05 | −210.96 | 92.03 | −210.00 |
| SD | 58.960 | 78.386 | 77.622 | 104.690 |

### Example 1-3-5. Clinical Response Evaluation According to CDAI-70 and CDAI-Loo Response Criteria

As shown in the following Table 9, the proportion of patients who achieving clinical response according to the CDAI-7 was broadly comparable until Week 30 in the IV5 mg/kg treatment arm and the SC 120/240 mg treatment arm. The proportion of patients achieving clinical response according to the CDAI-70 was broadly similar between IV and SC treatment arms until Week 54 excluding Week 46 after the IV 5 mg/kg was switched to the SC 120/240 mg at Week 30. The three patients of the IV 5 mg/kg treatment arm showed a temporary increase in CDAI scores and did not show CDAI-70 responses at Week 46, but regained CDAI-70 response at Week 54. The response evaluation according to the CDAI-100 response criteria showed similar results to those of the CDAI-70.

TABLE 9

| | | |
|---|---|---|
| Items | IV 5 mg/kg | SC 120/240 mg |
| Visit | (N = 25) | (N = 28) |

CDAI-70
Week 2

| | | |
|---|---|---|
| Number of patients responded (%) | 14 (56.0) | 16 (57.1) |

Week 6

| | | |
|---|---|---|
| Number of patients responded (%) | 21 (84.0) | 21 (75.0) |

Week 14

| | | |
|---|---|---|
| Number of patients responded (%) | 22 (88.0) | 22 (78.6) |

Week 22

| | | |
|---|---|---|
| Number of patients responded (%) | 21 (84.0) | 22 (78.6) |

Week 30

| | | |
|---|---|---|
| Number of patients responded (%) | 17 (68.0) | 19 (67.9) |

Week 38

| | | |
|---|---|---|
| Number of patients responded (%) | 17 (68.0) | 21 (75.0) |

Week 46

| | | |
|---|---|---|
| Number of patients responded (%) | 14 (56.0) | 20 (71.4) |

Week 54

| | | |
|---|---|---|
| Number of patients responded (%) | 17 (68.0) | 20 (71.4) |

CDAI-100
Week 2

| | | |
|---|---|---|
| Number of patients responded (%) | 9 (36.0) | 13 (46.4) |

Week 6

| | | |
|---|---|---|
| Number of patients responded (%) | 17 (68.0) | 16 (57.1) |

Week 14

| | | |
|---|---|---|
| Number of patients responded (%) | 18 (72.0) | 19 (67.9) |

Week 22

| | | |
|---|---|---|
| Number of patients responded (%) | 20 (80.0) | 21 (75.0) |

Week 30

| | | |
|---|---|---|
| Number of patients responded (%) | 16 (64.0) | 19 (67.9) |

Week 38

| | | |
|---|---|---|
| Number of patients responded (%) | 16 (64.0) | 20 (71.4) |

Week 46

| | | |
|---|---|---|
| Number of patients responded (%) | 13 (52.0) | 19 (67.9) |

Week 54

| | | |
|---|---|---|
| Number of patients responded (%) | 16 (64.0) | 18 (64.3) |

### Example 1-3-6. Clinical Remission Evaluation by CDAI

As shown in the following Table 10, the proportion of patients achieving a clinical remission by the CDAI score was generally comparable between the IV 5 mg/kg treatment arm and the SC 120/240 mg treatment arm until Week 30. A clinical remission rate was generally similar between the two treatment arms until Week 54 after IV 5 mg/kg was switched to the SC 120/240 mg at Week 30 and a slightly higher clinical remission rate was shown in the SC 120/240 mg treatment arm at Week 46. This result was in line with the result of clinical response of the three patients of the IV mg/kg treatment arm who showed a temporary increase in CDAI scores at Week 46, subsequently, those patients did not achieve clinical remission at Week 46 as well. Such patients regained the clinical remission according to the CDAI score at Week 54

TABLE 10

| | | |
|---|---|---|
| | | IV 5 mg/kg and |
| | IV 5 mg/kg | SC 120/240 mg |
| Visit | (N = 25) | (N = 28) |

| | |
|---|---|
| Statistics | Number of patients (%) |

Week 2

| | | |
|---|---|---|
| Number of patients achieved (%) | 7 (28.0) | 8 (28.6) |

Week 6

| | | |
|---|---|---|
| Number of patients achieved (%) | 12 (48.0) | 14 (50.0) |

Week 14

| | | |
|---|---|---|
| Number of patients achieved (%) | 16 (64.0) | 16 (57.1) |

Week 22

| | | |
|---|---|---|
| Number of patients achieved (%) | 15 (60.0) | 17 (60.7) |

Week 30

| | | |
|---|---|---|
| Number of patients achieved (%) | 14 (56.0) | 18 (64.3) |

Week 38

| | | |
|---|---|---|
| Number of patients achieved (%) | 15 (60.0) | 17 (60.7) |

Week 46

| | | |
|---|---|---|
| Number of patients achieved (%) | 12 (48.0) | 17 (60.7) |

Week 54

| | | |
|---|---|---|
| Number of patients achieved (%) | 14 (56.0) | 16 (57.1) |

### Example 1-3-7. Disease Activity Index Measured by Mayo Scoring System (Patients with Ulcerative Colitis)

As shown in the following Table 11, there was a trend that a mean of total and partial Mayo scores was similar at baseline between the IV5 mg/kg treatment arm and the SC 120/240 mg treatment arm. Further, there was a slightly higher improvement in total and partial Mayo scores from the SC 120/240 mg treatment arm at Week 22. However, it was identified that the mean and the change from baseline of total and partial Mayo scores became similar between the two treatment arms at Week 54 after the of the IV 5 mg/kg was switched to the SC 120/240 mg at Week 30.

TABLE 11

| | |
|---|---|
| IV 5 mg/kg | SC 120/240 mg |
| (N = 39) | (N = 38) |

| | | | | |
|---|---|---|---|---|
| Visit | Actual | Change from | Actual | Change from |
| Statistics | Result | Baseline | Result | Baseline |

Total Mayo Score
Baseline

| | | |
|---|---|---|
| Number | 39 | 38 |
| Mean | 8.3 | 7.9 |
| SD | 1.34 | 1.41 |

Week 22

| | | | | |
|---|---|---|---|---|
| Number | 28 | 28 | 34 | 34 |
| Mean | 3.8 | −4.4 | 2.5 | −5.3 |
| SD | 3.03 | 2.85 | 2.34 | 2.02 |

Week 54

| | | | | |
|---|---|---|---|---|
| Number | 31 | 31 | 30 | 30 |
| Mean | 1.9 | −6.2 | 1.7 | −6.0 |
| SD | 2.26 | 2.88 | 2.22 | 1.90 |

Partial Mayo Score
Baseline

| | | |
|---|---|---|
| Number | 39 | 38 |
| Mean | 5.9 | 5.4 |
| SD | 1.21 | 1.31 |

Week 2

| | | | | |
|---|---|---|---|---|
| Number | 39 | 39 | 38 | 38 |
| Mean | 3.3 | −2.6 | 3.3 | −2.1 |
| SD | 1.82 | 1.79 | 2.18 | 2.05 |

Week 6

| | | | | |
|---|---|---|---|---|
| Number | 39 | 39 | 38 | 38 |
| Mean | 2.5 | −3.4 | 2.6 | −2.9 |
| SD | 1.74 | 1.80 | 2.13 | 2.06 |

Week 14

| | | | | |
|---|---|---|---|---|
| Number | 39 | 39 | 36 | 36 |
| Mean | 2.3 | −3.6 | 1.7 | −3.7 |
| SD | 1.92 | 2.11 | 1.62 | 1.73 |

Week 22

| | | | | |
|---|---|---|---|---|
| Number | 36 | 36 | 35 | 35 |
| Mean | 2.3 | −3.5 | 1.3 | −4.0 |
| SD | 1.97 | 1.93 | 1.63 | 1.55 |

Week 30

| | | | | |
|---|---|---|---|---|
| Number | 36 | 36 | 35 | 35 |
| Mean | 1.9 | −3.9 | 1.2 | −4.1 |
| SD | 1.88 | 2.06 | 1.59 | 1.78 |

Week 38

| | | | | |
|---|---|---|---|---|
| Number | 34 | 34 | 35 | 35 |
| Mean | 1.4 | −4.4 | 1.2 | −4.1 |
| SD | 1.39 | 1.65 | 1.55 | 1.88 |

Week 46

| | | | | |
|---|---|---|---|---|
| Number | 33 | 33 | 34 | 34 |
| Mean | 0.9 | −4.8 | 1.1 | −4.3 |
| SD | 1.22 | 1.89 | 1.48 | 1.85 |

Week 54

| | | | | |
|---|---|---|---|---|
| Number | 32 | 32 | 32 | 32 |
| Mean | 1.0 | −4.7 | 0.9 | −4.5 |
| SD | 1.60 | 2.22 | 1.31 | 1.27 |

### Example 1-3-8. Clinical Response Evaluation Measured by Mayo Scoring System

As shown in the following Table 12, the proportion of patients achieving clinical response according to total Mayo score was higher in the SC 120/240 mg treatment arm than in the IV 4 mg/kg treatment arm at Week 22. Patients who discontinued the study before Week 22 or patients who did not receive endoscopy at Week 22 were 4 (10.5%) in the SC 120/240 mg treatment arm and 11 (28.2%) in the IV 5 mg/kg treatment arm. A higher missing rate was shown in the IV treatment arm, thus showing a relatively lower clinical response rate based on total Mayo scores at Week 22. The clinical response rate based on total Mayo scores was similar between the two treatment arms at Week 54 after the IV 5 mg/kg was switched to the SC 120/240 mg at Week 30.

The proportion of patients achieving clinical response according to partial Mayo score was broadly similar between the two treatment arms until Week 22 and a slightly higher response rate was observed in the SC treatment arm at Week 30. There was a trend that the clinical response rate based on partial Mayo scores was broadly comparable similar between the two treatment arms until Week 54 after the IV 5 mg/kg was switched to the SC 120/240 mg at Week 30.

TABLE 12

| | | |
|---|---|---|
| Parameter | IV 5 mg/kg | SC 120/240 mg |
| Visit | (N = 39) | (N = 38) |

Total Mayo score
Week 22

| | | |
|---|---|---|
| Number of patients responded (%) | 21 (53.8) | 30 (78.9) |

Week 54

| | | |
|---|---|---|
| Number of patients responded (%) | 27 (69.2) | 27 (71.1) |

Partial Mayo score
Week 2

| | | |
|---|---|---|
| Number of patients responded (%) | 25 (64.1) | 20 (52.6) |

Week 6

| | | |
|---|---|---|
| Number of patients responded (%) | 31 (79.5) | 28 (73.7) |

Week 14

| | | |
|---|---|---|
| Number of patients responded (%) | 33 (84.6) | 30 (78.9) |

Week 22

| | | |
|---|---|---|
| Number of patients responded (%) | 30 (76.9) | 32 (84.2) |

Week 30

| | | |
|---|---|---|
| Number of patients responded (%) | 29 (74.4) | 33 (86.8) |

Week 38

| | | |
|---|---|---|
| Number of patients responded (%) | 31 (79.5) | 33 (86.8) |

Week 46

| | | |
|---|---|---|
| Number of patients responded (%) | 30 (76.9) | 32 (84.2) |

Week 54

| | | |
|---|---|---|
| Number of patients responded (%) | 28 (71.8) | 31 (81.6) |

### Example 1-3-9. Clinical Remission Evaluation by Mayo Score (Patients with Ulcerative Colitis)

As shown in the following Table 13, the proportion of patients achieving clinical remission according to total Mayo score was higher in the SC 120/240 mg treatment arm than in the IV 5 mg/kg treatment arm at Week 22. Patients who discontinued the study before Week 22 or patients who did not receive endoscopy at Week 22 were 4 (10.5%) in the SC 120/240 mg treatment arm and 11 (28.2%) in the IV 5 mg/kg treatment arm. A higher missing rate was shown in the IV treatment arm, thus showing a relatively lower clinical remission rate based on total Mayo scores at Week 22. The clinical remission rate based on total Mayo scores was similar between the two treatment arms at Week 54 after the IV 5 mg/kg was switched to the SC 120/240 mg at Week 30.

There was a trend that the proportion of patients achieving the clinical remission based on partial Mayo scores was broadly similar between the two treatment arms until Week 30 and a slightly higher clinical remission rate was observed in the SC 120/240 mg treatment arm than in the IV 5 mg/kg treatment arm at Week 22. There was a trend that the clinical remission rate based on partial Mayo scores was similar between the two treatment arms until Week 54 after the IV 5 mg/kg was switched to the SC 120/240 mg at Week 30.

TABLE 13

| | | |
|---|---|---|
| Parameter | IV 5 mg/kg | SC 120/240 mg |
| Visit | (N = 39) | (N = 38) |

Total Mayo score
Week 22

| | | |
|---|---|---|
| Number of patients achieved (%) | 10 (25.6) | 20 (52.6) |

Week 54

| | | |
|---|---|---|
| Number of patients achieved (%) | 21 (53.8) | 21 (55.3) |

Partial Mayo score
Week 2

| | | |
|---|---|---|
| Number of patients achieved (%) | 7 (17.9) | 9 (23.7) |

Week 6

| | | |
|---|---|---|
| Number of patients achieved (%) | 12 (30.8) | 14 (36.8) |

Week 14

| | | |
|---|---|---|
| Number of patients achieved (%) | 17 (43.6) | 17 (44.7) |

Week 22

| | | |
|---|---|---|
| Number of patients achieved (%) | 15 (38.5) | 23 (60.5) |

Week 30

| | | |
|---|---|---|
| Number of patients achieved (%) | 21 (53.8) | 26 (68.4) |

Week 38

| | | |
|---|---|---|
| Number of patients achieved (%) | 25 (64.1) | 26 (68.4) |

Week 46

| | | |
|---|---|---|
| Number of patients achieved (%) | 26 (66.7) | 26 (68.4) |

Week 54

| | | |
|---|---|---|
| Number of patients achieved (%) | 24 (61.5) | 26 (68.4) |

### Example 1-3-10. Efficacy Evaluation by Mucosal Healing (Patients with Ulcerative Colitis)

As shown in the following Table 14, the proportion of patients achieving mucosal healing was higher in the SC 120/240 mg treatment arm than in the IV 5 mg/kg treatment arm at Week 22. Patients who discontinued the study before Week 22 or patients who did not receive endoscopy at Week 22 were 4 (10.5%) in the SC 120/240 mg treatment arm and 11 (28.2%) in the IV 5 mg/kg treatment arm. A higher missing rate was shown in the IV treatment arm, thus showing a relatively lower mucosal healing rate at Week 22. The proportion of patients achieving mucosal healing was similar between the two treatment arms at Week 54 after the IV 5 mg/kg was switched to the SC 120/240 mg at Week 30, thus showing that treatment with the SC 120/240 mg was also efficacious in the IV 5 mg/kg treatment arm.

TABLE 14

| | |
|---|---|
| IV 5 mg/kg | SC 120/240 mg |
| (N = 39) | (N = 38) |

| | |
|---|---|
| Visit | Number of patients (%) |

| | | |
|---|---|---|
| Week 22 | 15 (38.5) | 23 (60.5) |
| Week 54 | 25 (64.1) | 24 (63.2) |

Pharmacokinetic Evaluation

### Example 1-3-11. Pharmacokinetic Parameters

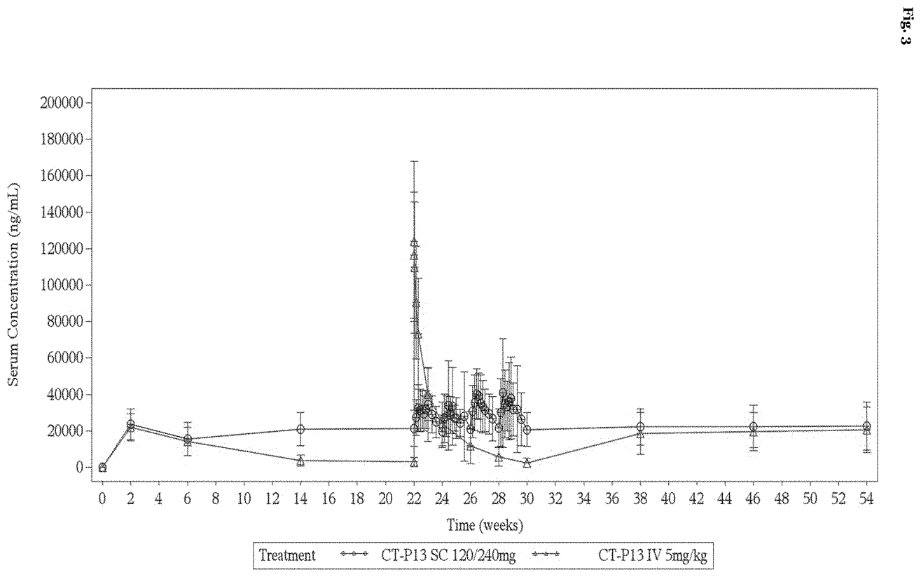

As shown in FIG. 3 and the following Table 15, the mean pre-dose serum concentration of infliximab was similar between the two treatment arms from Week 0 to 6 after the administration of the infliximab IV 5 mg/kg at Weeks 0 and 2. From the maintenance phase, the mean pre-dose serum concentration in the SC 120/240 mg treatment arm gradually increased from Week 6 to Week 14 and maintained consistent level from Week 14 to Week 22 as a result of the 2-week dosing interval of infliximab SC. The mean pre-dose serum concentration in the IV 5 mg/kg treatment arm gradually decreased from Week 6 to Week 14 and generally maintained consistent level from Week 14 to Week 30 as a result of the 8-week dosing interval of infliximab IV. The mean pre-dose serum concentration levels during this period were consistently higher in the SC 120/240 mg treatment arm compared to the IV 5 mg/kg treatment arm.

The mean pre-dose serum concentration of infliximab continued to increase after the IV 5 mg/kg was switched to the SC 120/240 mg at Week 30. At Week 38, such mean exceeded a target therapeutic serum concentration (5 μg/ml) and reached the serum concentration of infliximab similar to that of the SC 120/240 mg treatment arm and was maintained consistent level up to Week 54.

TABLE 15

| | | |
|---|---|---|
| Parameter | | |
| Visit | IV 5 mg/kg | SC 120/240 mg |
| Statistics | (N = 64) | (N = 63) |

Predicted AUC_{τ} (μg*h/ml)

Week 22

| | | |
|---|---|---|
| Number of Patients | 57 | 14 |

| | | | |
|---|---|---|---|
| Mean (CV %) | 28179.3 | (35.8) | 8275.7 (30.5) |

| | | |
|---|---|---|
| Week 24 | | |
| Number of Patients | N/A | 14 |
| Mean (CV %) | N/A | 7837.1 (28.1) |
| Week 26 | | |
| Number of Patients | N/A | 15 |
| Mean (CV %) | N/A | 9870.7 (25.4) |
| Week 28 | | |
| Number of Patients | N/A | 15 |
| Mean (CV %) | N/A | 9410.0 (42.9) |

Predicted AUC_{ss8 w} (μg*h/ml)

| | | |
|---|---|---|
| Week 22 | | |
| Number of Patients | 57 | 58 |

| | | | |
|---|---|---|---|
| Mean (CV %) | 28284.0 | (36.3) | 35467.2 (33.8) |

Predicted C_{trough} (μg/ml)

| | | |
|---|---|---|
| Week 22 | | |
| Number of Patients | 57 | 28 |

| | | | |
|---|---|---|---|
| Mean (CV %) | 2.6433 | (78.0391) | 18.7429 (38.7052) |

| | | |
|---|---|---|
| Week 24 | | |
| Number of Patients | N/A | 30 |
| Mean (CV %) | N/A | 19.8593 (34.6997) |
| Week 26 | | |
| Number of Patients | N/A | 30 |
| Mean (CV %) | N/A | 21.3967 (43.6753) |
| Week 28 | | |
| Number of Patients | N/A | 58 |
| Mean (CV %) | N/A | 20.1697 (44.9494) |

Predicted C_{max, ss} (μg/ml)

| | | |
|---|---|---|
| Week 22 | | |
| Number of Patients | 57 | 14 |

| | | | |
|---|---|---|---|
| Mean (CV %) | 105.58 | (21.21) | 29.80 (26.71) |

| | | |
|---|---|---|
| Week 24 | | |
| Number of Patients | N/A | 14 |
| Mean (CV %) | N/A | 27.09 (27.07) |
| Week 26 | | |
| Number of Patients | N/A | 15 |
| Mean (CV %) | N/A | 33.57 (25.35) |
| Week 28 | | |
| Number of Patients | N/A | 15 |
| Mean (CV %) | N/A | 30.63 (41.17) |

*Abbreviation: AUC_{τ}: Model predicted area under the concentration-time curve at steady state (Weeks 22-30), Cₘₐₓ: Model predicted maximum serum concentration, C_{trough}: Model predicted trough serum concentration, and CV %: Percent coefficient of variation.
** Patients from IV 5 mg/kg treatment arm were administered the infliximab every 8 weeks, while patients from SC 120/240 mg treatment arm were administered the infliximab every 2 weeks.

Thus, the PK parameters of IV 5 mg/kg treatment arm were obtained at Week 22 and those of the SC 120/240 mg treatment arm were obtained for Weeks 22, 24, 26 and 28.

### Example 2. Evaluation of Safety and Therapeutic Efficacy on Subcutaneous Administration of Infliximab to Patients with Rheumatoid Arthritis (RA) (Study 3.5)

### Example 2-1. Study Protocol

The present study of infliximab (CT-P13) was a randomized, multi-center, parallel-group and phase I/III trial, designed to evaluate pharmacokinetics, efficacy and safety between the infliximab for subcutaneous administration (hereinafter infliximab SC) and the infliximab for intravenous administration (hereinafter infliximab IV) in combination with methotrexate (MTX) and folic acid in patients with active rheumatoid arthritis, who had not shown an adequate response to MTX-only therapy over the three months or longer, in which the present study was composed of two parts.

Part 1 was designed to identify an optimal dose of the infliximab SC, in which the optimal dose of the infliximab SC corresponding to 3 mg/kg of the infliximab IV over the first 30 weeks was identified by means of an area under the concentration-time curve (AUC_{τ}) at steady state between Weeks 22 and 30. In case of Part 1, a study period lasted for a maximum of 65 weeks, including a duration from Screening (the maximum of 3 weeks) to End-of-Study visit.

Part 2 was designed to demonstrate the non-inferiority of efficacy between the infliximab SC and the infliximab IV. Thus, it might be demonstrated that the infliximab SC was not inferior to the infliximab IV in terms of efficacy, determined by clinical response according to mean change from baseline of DAS28 (Disease Activity Score in 28 joints) (C-reactive protein, CRP) using 28 joints at Week 22. In case of Part 2, the administered dose and dosing interval of the infliximab SC were set to the administration of 120 mg every 2 weeks.

Part 1

Patients had to meet all the following inclusion criteria to be enrolled in this study.
Patient who suffered from an active disease defined by the presence of at least 6 or more swollen joints and tender joints out of 28 joints, and a serum concentration of C-reactive protein (CRP) >0.6 mg/dL; and
Patient who had been treated with methotrexate at a dose of 12.5-25 mg/week (or 10-25 mg/week for patients in South Korea) for at least 3 months before the administration of the test drug (Day 0) and had been treated at the same dose for the last 4 weeks prior to the first administration of the study drug.

Patients meeting any of the following criteria were excluded from this study.
Patient who had previously received a biological agent for the treatment of the RA and/or a TNFα inhibitor for the treatment of other disease;
Patient who had allergies to any of the excipients of infliximab or any other murine and/or human proteins, or patient who had a hypersensitivity to immunoglobulin products; The present study was composed of three study periods: Screening; Treatment Period; and End-of-Study. The Screening was carried out between Days −21 and −1 before an initial administration of the study drug, in which the eligibility of patients for study was evaluated. All the assessments including hepatitis B, hepatitis C and human immunodeficiency virus (HIV-1 and HIV-2) infections status, a urine and serum pregnancy test for women of childbearing potential, rheumatoid factor, anti-cyclic citrullinated peptide, 12-lead ECG, clinical laboratory tests, etc., were carried out. Also, an interferon-gamma release assay (IGRA) and a chest X-ray examination were performed so as to exclude patients with TB.

All the patients enrolled into the study received a single dose of the infliximab IV at Weeks 0 and 2, respectively. Further, methotrexate with folic acid was co-administered to minimise or prevent AEs related to MTX side effects, in which patients were also reminded of taking a maintenance dose of the MTX from beginning to end of study. In addition, a patient was also able to be premedicated 30 to 60 minutes prior to the start of study drug administration and any premedications such as but not limited to antihistamine (at equivalent dose of 2 to 4 mg of chlorpheniramine), hydrocortisone, paracetamol, and/or nonsedating antihistamine (at equivalent dose of 10 mg of cetirizine) was able to be given at the investigator's discretion.

Those who received two full doses of the study drug and deemed to have no safety concern at the investigator's discretion, were randomly assigned to the SC 120 mg and IV 3 mg/kg treatment arms before treatment on Day 42, Week 6. The randomisation for random assignment was stratified by country, a serum CRP concentration for 2 weeks (0.6 mg/dl or less, or more than 0.6 mg/dl), and a weight (70 kg or less, or more than 70 kg) at Week 6. A total of 50 patients with active RA were enrolled, out of which 48 ones were randomly assigned to four study cohorts at a ratio of 1:1:1:1, in which the study drug was administered up to Week 54 (Table 16).

TABLE 16

| | | | | Dosage, | |
|---|---|---|---|---|---|
| | | | | Investigational | |
| | | | Investigational | Product, Method | Number of |
| Cohort Number | Dosage | | Product | of Administration | Patients |
| Cohort 1 | 3 | mg/kg | infliximab IV | Two hours IV infusion | 13 |
| | | | 100 mg/vial | | |
| Cohort 2 | 90 | mg | infliximab SC | Single SC injection | 11 |
| | | | 90 mg/PFS* | | |
| Cohort 3 | 120 | mg | infliximab SC | Single SC injection | 12 |
| | | | 120 mg/PFS | | |
| Cohort 4 | 180 | mg | infliximab SC | Double SC injections | 12 |
| | | | 90 mg/PFS | | |

*PFS, Pre-filled syringe

Those who were assigned to Cohort 1 received additional seven doses of the infliximab IV at Week 6 and subsequently every 8 weeks (Weeks 14, 22, 30, 38, 46 and 54). Those who were assigned to Cohorts 2, 3 and 4 were initially dosed with the infliximab SC at Week 6, and then additionally dosed with the infliximab SC every 2 weeks until Week 54. The initial dose assigned to all the patients of Cohorts 2, 3 and 4 was adjusted to an optimal dose after the optimal dose was confirmed through dose finding. After that, an additional SC injection using the optimal dose was performed until Week 54. The infliximab SC was injected into patients by a healthcare provider at each study center visit (Weeks 6, 8, 10, 14, 22, 24, 26, 28, 30, 38, 46 and 54). After proper training in injection technique, patients could self-inject with infliximab SC in all the other weeks (Weeks 12, 16, 18, 20, 32, 34, 36, 40, 42, 44, 48, 50 and 52), if the investigator determined that it was appropriate.

Patients returned to the study center at predefined time intervals for clinical assessments and blood sampling. At each visit, the patients were asked questions about adverse events (AE) and concomitant medications, while being monitored for clinical signs and symptoms of tuberculosis (TB). The evaluation of primary pharmacokinetic endpoint was performed at steady state between Weeks 22 and 30, then the evaluation of secondary pharmacokinetic endpoints was performed during the treatment period until Week 54, and then blood sampling for analysis as well as the evaluation of efficacy, PD and safety were respectively performed at a point of time specified in an evaluation schedule.

The End-of-Study visit was performed at the end of the maintenance phase or in 8 weeks after the last day of administration when patients were dropped out. Every effort was made to complete all the end-of-study evaluations at a time point of 8 weeks after the last administration to patients.

Part 2

Part 2 commenced based on a review by the independent Data Safety Monitoring Board (DSMB) with regard to PK modeling report data including PK, efficacy, PD and safety data, which were identified over the first 30 weeks in Part 1.

Part 2 was composed of three study periods including Screening; Treatment Period with a double-blinded period until Week 30 followed by an open-label period of 24 weeks; and End-of-Study. The Screening was carried out between Days −42 and 0 prior to the first administration of the study drug, in which the eligibility of patients for study was evaluated. All the examinations including hepatitis B, hepatitis C and human immunodeficiency virus (HIV-1 and HIV-2) infections, a urine and serum pregnancy test for women of childbearing potential, rheumatoid factor, anti-cyclic citrullinated peptide, 12-lead ECG, clinical laboratory tests, etc., were carried out. Also, an interferon-gamma release assay (IGRA) and a chest X-ray examination were performed so as to exclude patients with TB.

All the patients enrolled into the study initially received the infliximab IV at Weeks 0 and 2, respectively. Further, methotrexate with folic acid was co-administered to minimise or prevent AEs related to MTX side effects, in which patients were also reminded of taking a maintenance dose of the MTX from beginning to end of study. A patient was also able to be premedicated 30 to 60 minutes prior to the start of study drug administration and any premedications such as but not limited to antihistamine (at equivalent dose of 2 to 4 mg of chlorpheniramine), hydrocortisone, paracetamol, and/or nonsedating antihistamine (at equivalent dose of 10 mg of cetirizine) was able to be given at the investigator's discretion.

Those who received two full doses of the study drug twice and deemed to have no safety concern at the investigator's discretion, were randomly assigned to receive either the infliximab SC via a pre-filled syringe (PFS) and the placebo IV, or the infliximab IV and the placebo SC (PFS) before treatment at Week 6 and Day 42. The randomisation was stratified by country, a serum CRP concentration for 2 weeks (0.6 mg/dl or less, or more than 0.6 mg/dl), and a weight (100 kg or less, or more than 100 kg) at Week 6. A total of 357 patients with active RA were enrolled, out of which 343 ones were randomly assigned to two study treatment arms at a ratio of 1:1, in which the administration of the study drug was performed until Week 54. Further, a double placebo design was used to maintain blindness until Week 30 (Table 17).

TABLE 17

| Treatment | | | Arm number, Dosage, | | |
|---|---|---|---|---|---|
| Arm | Administered | | Investigational | Administration | Number of |
| Number | Dose | | Product | Method | Patients |
| Treatment | 3 | mg/kg | infliximab IV | Two hours IV | 176 |
| Arm 1 | | | 100 mg/vial | infusion | |
| Treatment | 120 | mg | infliximab SC | Single SC | 167 |
| Arm 2 | | | 120 mg/PFS | injection | |

* PFS, Pre-filled syringe

Those who were assigned to Treatment Arm 1 received additional three doses of the infliximab IV at Week 6 and subsequently every 8 weeks (Weeks 14 and 22) until Week 22, while the placebo SC was administered a Week 6 and subsequently every 2 weeks until Week 28. After that, the IV 3 mg/kg was switched to the SC 120 mg (PFS) at Week 30. Then, the infliximab SC (PFS) was administered until Week 54. Those who were assigned to Treatment Arm 2 were initially dosed with the infliximab SC (PFS) at Week 6 and subsequently every 2 weeks, which continued until Week 54, while the placebo IV was administered at Weeks 6, 14 and 22.

The infliximab SC (the placebo SC during a double-blind period) was injected into patients by a healthcare professional at each study center visit (Weeks 6, 14, 22, 24-28 (those who visited for PK evaluation), 30, 38, 46 and 54). However, in all the other weeks (Weeks 8, 10, 12, 16, 18, 20, 24-28 (those who did not visit for PK evaluation), 32, 34, 36, 40, 42, 44, 48, 50 and 52), patients were allowed to perform a self-injection of the infliximab SC (the placebo SC during the double-blind period), if the investigator determined it as suitable after training them for appropriate injection techniques.

In a certain country, the infliximab SC was self-injected by an auto-injector (AI) every 2 weeks from Week 46 to 54, and then was switched to the self-injection of the infliximab SC (PFS) from Week 56 to 64. Evaluations by a self-injection assessment questionnaire before and after, a self-injection assessment checklist, and a potential hazards checklist were performed so that the usability of the infliximab SC (AI) might be evaluated.

In case of Part 2, clinical evaluation, blood sampling and study visits for each type were performed in the same way as shown in Part 1 as well as at a time point specified in an evaluation schedule.

### Example 2-2. Efficacy Evaluation Through PK-PD Modeling

A simulation was performed to evaluate pharmacokinetics and efficacy (DAS28) in a cohort of RA patients, when 120 mg of infliximab (CT-P13) was subcutaneously administered at an interval of 2 weeks in an induction phase (Weeks 0 and 2) without an infliximab IV administration. A profile of pharmacokinetics and efficacy (DAS28) was compared IV 3 mg/kg cohort administered with infliximab IV at Week 0 and 2 and SC 120 mg experimental cohort administered with SC 120 mg every 2 weeks from Week 0. At last, C_{trough} at steady state and efficacy (DAS28) was compared between the two treatment cohorts.

The present PK-PD modeling for CT-P13 was performed based on study data on CT-P13 SC for RA patients, which had been previously performed. Specifically, the data used for PK and PK-PD analysis modeling were developed based on the data obtained from seven clinical studies in different cohorts. The present PK-PD modeling was performed with a 2-compartment PK model, which reflects weights and an effect of emerging immune responses, in a time-dependent manner. A final PD model is an indirect-response model to identify an effect of infliximab SC on suppressing DAS28 responses. A PK-PD modeling simulation obtained from RA patients includes a population PK-PD analysis which uses data of CT-P13 3.1 study and CT-P13 3.5 study (n=992).

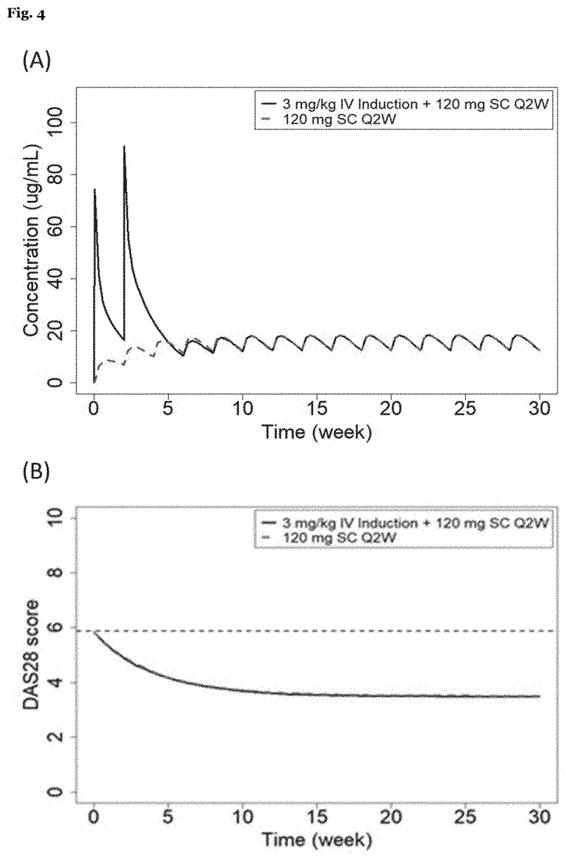

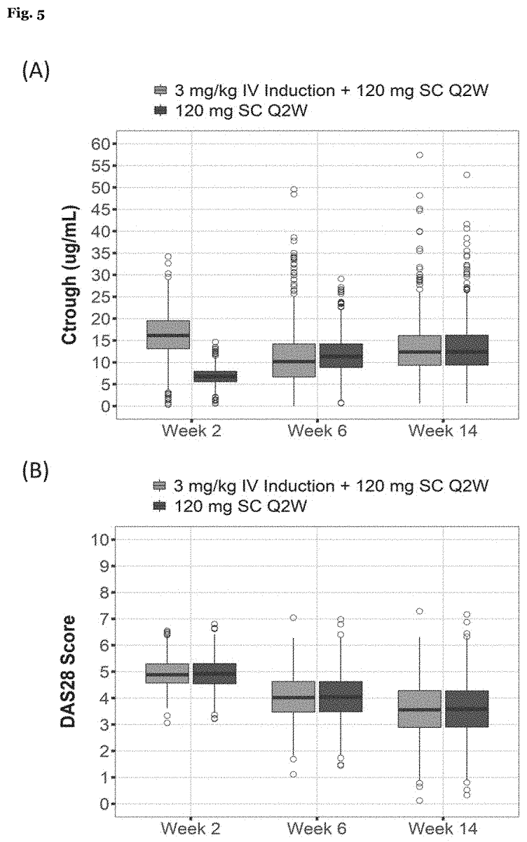

Thus, as shown in FIGS. 4 and 5, the present PK-PD modeling results showed that there is no significant difference between two treatment cohorts in terms of C_{trough} at steady state and efficacy (DAS28). In the cohort dosed with 120 mg of infliximab SC every 2 weeks from Week 0, it was measured that C_{trough} of median is higher than a target therapeutic serum concentration, i.e., 1 μg/ml. It was demonstrated identified through modeling that efficacy is similar between the two dosing regimens (DAS28) and the target serum concentration is achieved, and it was also confirmed that infliximab SC 120 mg every 2 weeks is an optimal dose for RA patients.

### Example 2-3. Actual Clinical Results (Study 3.5 Part 2)

Safety Evaluation

### Example 2-3-1. Summary of Adverse Events

The safety assessments were secondary endpoints and were performed on immunogenicity, hypersensitivity monitoring (including delayed hypersensitivity monitoring), measurement of vital signs (including blood pressure, heart and respiratory rates, and body temperature), weight, interferon-gamma release assay, chest X-ray, hepatitis B, hepatitis C and human immunodeficiency virus (HIV1 and HIV-2) infectious status, findings on physical examination, 12-lead ECG, adverse events (including serious adverse events), adverse events of special interest (infusion-related reaction/hypersensitivity reaction/anaphylactic reaction [administration-related reaction], delayed hypersensitivity reaction, injection site reaction, infection and malignancies), signs and symptoms of tuberculosis, clinical laboratory analysis, pregnancy test, prior and concomitant medications, local site pains using a 100 mm visual analogue scale (VAS).

The cumulative safety data included the treatment-emergent adverse events (TEAEs) (and serious adverse events) regardless of correlations with the study drug until the End-of-Study visit, in which an overall summary of the TEAEs during a maintenance phase (Weeks 6 to 64) was presented in Table 18. In general, 622 TEAEs were occurred in 209 patients (60.9%)-117 patients (66.9%) from the IV 3 mg/kg treatment arm and 92 patients (54.8%) from the SC 120 mg treatment arm respectively, thus indicating a similar proportion between the two treatment arms. Also, the majority of TEAEs were grade 1 or 2 in intensity. Out of all the adverse events, TEAEs reported in a total of 145 patients (42.3%) were regarded to be related to the study drug by the investigator.

The treatment-emergent serious adverse events (TESAEs) were occurred in 19 patients (5.5%)-13 patients (7.4%) from the IV 3 mg/kg treatment arm and 6 patients (3.6%) from the SC 120 mg treatment arm, respectively. The intensity of majority TESAEs was shown as a grade 3 or lower, out of which TESAEs regarded to be related to the study drug by the investigator were reported in 4 patients (2.3%) from the IV 3 mg/kg treatment arm and 3 patients (1.8%) from the SC 120 mg treatment arm. Also, out of all the TESAEs, the permanent study drug discontinuation according to the investigator's decision was reported in a total of 20 patients (5.8%) (14 patients (8.0%) from the IV 3 mg/kg treatment arm; 6 patients (3.6%) from the SC 120 mg treatment arm).

Out of the TEAEs classified as administration-related reaction including an infusion-related reaction (IRR) and a systemic injection reaction (SIR), hypersensitivity or anaphylactic reaction was reported in a total of 15 patients (4.4%)-10 patients (5.7%) from the IV 3 mg/kg treatment arm and 5 patients (3.0%) from the SC 120 mg treatment arm. Out of the patients reported as IRR (10 from IV 3 mg/kg; 2 from SC 120 mg), patients who had positive results for the anti-drug antibody (ADA) amounted to 6 in total, out of which 5 patients were reported to have positive results for the NAb. Out of 15 patients reported as administration-related reaction, 6 patients received premedication.

The TEAEs classified as infection were reported in 60 patients (34.3%) from the IV 3 mg/kg treatment arm and 49 patients (29.2%) in the SC 120 mg treatment arm.

TABLE 18

| | IV 3 mg/kg | SC 120 mg |
|---|---|---|
| | (N = 175) | (N = 168) |
| Number of patients with at least one TEAEs (%) | 117 (66.9) | 92 (54.8) |
| Related | 72 (41.1) | 73 (43.5) |
| Unrelated | 77 (44.0) | 46 (27.4) |
| Number of patients with at least one TEASEs (%) | 13 (7.4) | 6 (3.6) |
| Related | 4 (2.3) | 3 (1.8) |
| Unrelated | 11 (6.3) | 3 (1.8) |
| Number of patients with at least one TEAEs leading | 14 (8.0) | 6 (3.6) |
| to study drug discontinuation (%) | | |
| Number of patients with at least one TEAEs | 10 (5.7) | 5 (3.0) |
| classified as administration-related reaction (%) | | |
| Number of patients with at least one TEAEs | 22 (12.6) | 30 (17.9) |
| classified as injection site reaction (%) | | |
| Number of patients with at least one TEAEs | 60 (34.3) | 49 (29.2) |
| classified as infection (%) | | |

* At each level, patients were counted once, if they reported more than one event. Only the most severe event was counted. Each event was considered to be related, only if the relationship was defined as ‘Possible,’ ‘Probable’ or ‘Definite’.

### Example 2-3-2. Immunogenicity Evaluation

As shown in the following Table 19, the proportion of patients with positive ADA results in the SC 120 mg treatment arm was similar to or slightly lower than that of the IV 3 mg/kg treatment arm.

TABLE 19

| | IV 3 mg/kg | SC 120 mg |
|---|---|---|
| Visit, n (%) | (N = 175) | (N = 168) |
| Week 0 | | |
| ADA positive | 7 (4.0) | 4 (2.4) |
| NAb positive (% of number of | 2 (28.6) | 0 (0) |
| patients with positive ADA) | | |
| Week 6 | | |
| ADA positive | 14 (8.0) | 17 (10.1) |
| NAb positive (% of number of | 9 (64.3) | 9 (52.9) |
| patients with positive ADA) | | |
| Week 14 | | |
| ADA positive | 64 (36.6) | 52 (31.0) |
| NAb positive (% of number of | 59 (92.2) | 41 (78.8) |
| patients with positive ADA) | | |
| Week 22 | | |
| ADA positive | 104 (59.4) | 82 (48.8) |
| NAb positive (% of number of | 89 (85.6) | 57 (69.5) |
| patients with positive ADA) | | |
| Week 30 | | |
| ADA positive | 100 (57.1) | 82 (48.8) |
| NAb positive (% of number of | 84 (84.0) | 45 (54.9) |
| patients with positive ADA) | | |
| Week 38 | | |
| ADA positive | 95 (54.3) | 83 (49.4) |
| NAb positive (% of number of | 74 (77.9) | 52 (62.7) |
| patients with positive ADA) | | |
| Week 46 | | |
| ADA positive | 87 (49.7) | 75 (44.6) |
| NAb positive (% of number of | 70 (80.5) | 51 (68.0) |
| patients with positive ADA) | | |
| Week 54 | | |
| ADA positive | 79 (45.1) | 70 (41.7) |
| NAb positive (% of number of | 64 (81.0) | 47 (67.1) |
| patients with positive ADA) | | |
| Positive conversion after | | |
| Week 0 administaration** | | |
| ADA positive | 129 (73.7) | 114 (67.9) |
| NAb positive (% of number of | 112 (86.8) | 85 (74.6) |
| patients with positive ADA) | | |

* Abbreviation:
* ADA: anti-drug antibody; NAb: neutralizing antibody
**The patients who had ever been identified as positive for the ADA and the NAb even once until the End-of-Study visit after the first administration were used for the counting (however, the results for the ADA or the NAb before the administration (baseline visit) were not considered for the counting).

### Example 2-3-3. Local Site Pain Assessment Using the Visual Analogue Scale (VAS)

A range of the visual analogue scale (VAS) was from 0 to 100 mm, with higher scores indicating more severe pain. As shown in the following Table 20, a slightly higher level of VAS was observed in the SC 120 mg treatment arm at first time of infliximab SC administration (at Week 6). However, as the SC administration was performed repeatedly, the local site pain was gradually decreased and a similar level of the pain was reported in both treatment arms. The local site pain in the SC 120 mg treatment arm was decreased until Week 46. All the patients in the IV 3 mg/kg treatment arm were switched to the infliximab SC at Week 30, in which a higher level of the local site pain was observed from Week 30 than in the SC 120 mg treatment arm, but there was a trend that the level of the local site pain was gradually decreased until Week 46.

TABLE 20

| Visit | IV 3 mg/kg | SC 120 mg |
|---|---|---|
| Statistics | (N = 175) | (N = 168) |
| Week 6 | | |
| Number of Patients | 173 | 168 |
| Mean (SD) | 6.54 (12.139) | 10.29 (15.285) |
| Median | 2.00 | 4.10 |
| Minimum, Maximum | 0.0, 72.0 | 0.0, 81.0 |
| Week 14 | | |
| Number of Patients | 173 | 166 |
| Mean (SD) | 7.51 (14.719) | 7.30 (11.543) |
| Median | 3.00 | 3.00 |
| Minimum, Maximum | 0.0, 100.0 | 0.0, 73.0 |
| Week 30 | | |
| Number of Patients | 160 | 160 |
| Mean (SD) | 9.09 (14.752) | 7.40 (11.916) |
| Median | 3.00 | 3.00 |
| Minimum, Maximum | 0.0, 82.0 | 0.0, 88.0 |
| Week 38 | | |
| Number of Patients | 151 | 156 |
| Mean (SD) | 8.53 (13.464) | 7.19 (12.730) |
| Median | 3.00 | 3.00 |
| Minimum, Maximum | 0.0, 76.0 | 0.0, 100.0 |
| Week 46 | | |
| Number of Patients | 149 | 151 |
| Mean (SD) | 7.24 (12.072) | 6.50 (9.893) |
| Median | 2.10 | 3.0 |
| Minimum, Maximum | 0.0, 78.0 | 0.0, 61.0 |
| Week 54 | | |
| Number of Patients | 145 | 147 |
| Mean (SD) | 9.10 (15.276) | 7.92 (13.630) |
| Median | 3.00 | 3.00 |
| Minimum, Maximum | 0.0, 92.0 | 0.0, 79.0 |

Therapeutic Efficacy Evaluation

### Example 2-3-4. Disease Activity Index Measured by DAS28

As primary efficacy endpoints, a clinical response according to change from baseline by DAS28 (C reactive protein; CRP) at Week 22 was counted by using the ANCOVA (analysis of covariance), and it was demonstrated that SC 120 mg is non-inferior to IV 3 mg/kg. Least squares of disease activity index measured by DAS28 are summarized in the Table 21 and the actual values and the change from baseline are summarized in the Table 22.

TABLE 21

| | | | Difference | |
|---|---|---|---|---|
| | | | between | |
| | | | treatment arm | |
| | | Least | (estimate of | 95% |
| Treatment | Number of | Square | treatment | confidence |
| Arm | Patients | (SE) | difference) | interval |
| IV 3 mg/kg | 168 | 1.94 (0.209) | 0.27 | 0.02, 0.52 |
| SC 120 mg | 162 | 2.21 (0.221) | | |

TABLE 22

| | IV 3 mg/kg | | SC 120 mg | |
|---|---|---|---|---|
| Visit | Actual | Change from | Actual | Change from |
| Statistics | Result | Baseline | Result | Baseline |
| Baseline | | | | |
| Number of Patients | 174 | | 165 | |
| Mean (SD) | 5.863 (0.8090) | | 6.008 (0.7541) | |
| Week 2 | | | | |
| Number of Patients | 172 | 172 | 164 | 164 |
| Mean (SD) | 4.643 (1.0460) | −1.216 (1.0967) | 4.702 (0.9361) | −1.309 (0.9543) |
| Week 6 | | | | |
| Number of Patients | 174 | 174 | 165 | 165 |
| Mean (SD) | 4.112 (1.2105) | −1.751 (1.1498) | 3.983 (1.2021) | −2.026 (1.3484) |
| Week 14 | | | | |
| Number of Patients | 172 | 172 | 164 | 164 |
| Mean (SD) | 3.677 (1.2510) | −2.186 (1.2252) | 3.483 (1.1996) | −2.522 (1.3637) |
| Week 22 | | | | |
| Number of Patients | 168 | 168 | 162 | 162 |
| Mean (SD) | 3.482 (1.2329) | −2.390 (1.2716) | 3.338 (1.0958) | −2.662 (1.2599) |
| Week 30 | | | | |
| Number of Patients | 159 | 159 | 157 | 157 |
| Mean (SD) | 3.521 (1.2339) | −2.344 (1.2746) | 3.047 (1.1272) | −2.988 (1.3141) |
| Week 54 | | | | |
| Number of Patients | 145 | 145 | 145 | 145 |
| Mean (SD) | 2.913 (1.1648) | −2.939 (1.2678) | 2.796 (1.1414) | −3.243 (1.2855) |

### Example 2-3-5. ACR20, 50, 70 Response Evaluation

A proportion of patients who showed a clinical response according to the ACR20 (American College of Rheumatology) response evaluation was similar between the IV 3 mg/kg treatment arm and the SC 120 mg treatment arm until Week 22 (Table 23). However, a slightly higher response rate was shown in the SC 120 mg treatment arm at Week 30 (133 patients (76.4%) from the IV 3 mg/kg treatment arm; 142 patients (86.1%) from the SC 120 mg treatment arm). The response rate was slightly higher after the IV 3 mg/kg treatment arm was switched to the SC 120 mg at Week 30, but it was identified that the response rate tends to increase gradually as a whole. A similar trend was also identified in ACR50 and ACR70.

TABLE 23

| | | |
|---|---|---|
| | IV 3 mg/kg | SC 120 mg |
| Items | (N = 174) | (N = 165) |

| | |
|---|---|
| Visit | Number of patients (%) |

| | | |
|---|---|---|
| ACR20 | | |
| Week 2 | 57 (32.8) | 63 (38.2) |
| Week 6 | 103 (59.2) | 107 (64.8) |
| Week 14 | 130 (74.7) | 124 (75.2) |
| Week 22 | 137 (78.7) | 139 (84.2) |
| Week 30 | 133 (76.4) | 142 (86.1) |
| Week 54 | 125 (71.8) | 132 (80.0) |
| ACR50 | | |
| Week 2 | 19 (10.9) | 15 (9.1) |
| Week 6 | 45 (25.9) | 47 (28.5) |
| Week 14 | 73 (42.0) | 75 (45.5) |
| Week 22 | 90 (51.7) | 85 (51.5) |
| Week 30 | 87 (50.0) | 106 (64.2) |
| Week 54 | 101 (58.0) | 108 (65.5) |
| ACR70 | | |
| Week 2 | 7 (4.0) | 8 (4.8) |
| Week 6 | 18 (10.3) | 19 (11.5) |
| Week 14 | 40 (23.0) | 40 (24.2) |
| Week 22 | 49 (28.2) | 46 (27.9) |
| Week 30 | 47 (27.0) | 68 (41.2) |
| Week 54 | 68 (39.1) | 77 (46.7) |

### Example 2-3-6. EULAR Response Evaluation

A proportion of patients who showed a good or moderate to severe response in a European League Against Rheumatism (EULAR) response evaluation, which was classified based on DAS28 (CRP), was similar among respective treatment arms until Week 22, but was slightly higher in the SC 120 mg treatment arm at Week 30. However, a EULAR response rate at Week 54 was similar among respective treatment arms after the IV 3 mg/kg treatment arm was switched to the SC 120 mg at Week 30 (Table 24).

TABLE 24

| | | |
|---|---|---|
| | IV 3 mg/kg | SC 120 mg |
| Visit | (N = 174) | (N = 165) |

| | |
|---|---|
| Statistics | Number of patients (%) |

| | | |
|---|---|---|
| Week 2 | | |
| Number of patients | 103 (59.2) | 106 (64.2) |
| responded (%) | | |
| Moderate response | 85 (48.9) | 97 (58.8) |
| Good response | 18 (10.3) | 9 (5.5) |
| Week 6 | | |
| Number of patients | 139 (79.9) | 136 (82.4) |
| responded (%) | | |
| Moderate response | 94 (54.0) | 91 (55.2) |
| Good response | 45 (25.9) | 45 (27.3) |
| Week 14 | | |
| Number of patients | 149 (85.6) | 147 (89.1) |
| responded (%) | | |
| Moderate response | 86 (49.4) | 72 (43.6) |
| Good response | 63 (36.2) | 75 (45.5) |
| Week 22 | | |
| Number of patients | 156 (89.7) | 156 (94.5) |
| responded (%) | | |
| Moderate response | 91 (52.3) | 80 (48.5) |
| Good response | 65 (37.4) | 76 (46.1) |
| Week 30 | | |
| Number of patients | 145 (83.3) | 153 (92.7) |
| responded (%) | | |
| Moderate response | 83 (47.7) | 69 (41.8) |
| Good response | 62 (35.6) | 84 (50.9) |
| Week 54 | | |
| Number of patients | 142 (81.6) | 139 (84.2) |
| responded (%) | | |
| Moderate response | 57 (32.8) | 46 (27.9) |
| Good response | 85 (48.9) | 93 (56.4) |

Pharmacokinetic Evaluation

### Example 2-3-7. Pharmacokinetic Parameters

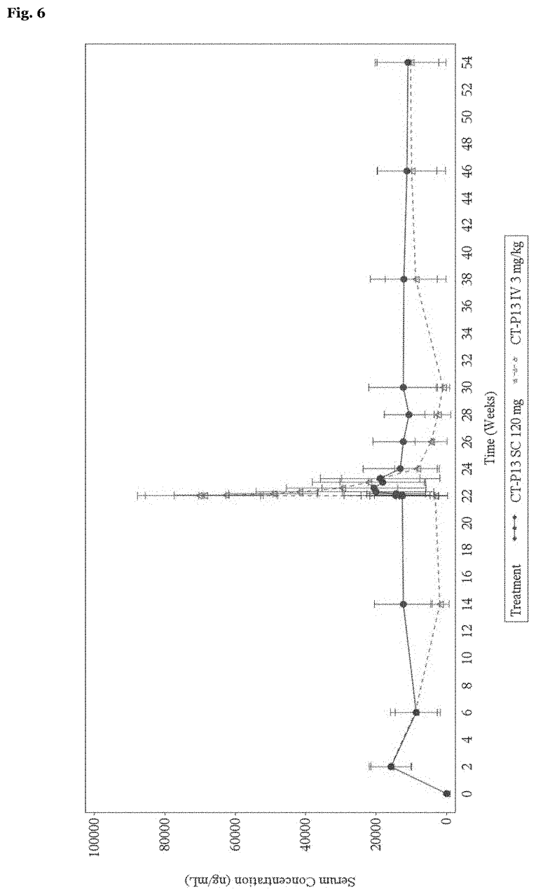

A mean serum concentrations before the administration of infliximab was similar between the treatment arms until Week 6 after the administration of the infliximab IV 3 mg/kg at Weeks 0 and 2. From the maintenance phase, the mean serum pre-dose concentration of infliximab was gradually increased until Week 14 in the SC 120 mg treatment arm at every other week, and then a constant concentration was maintained from Week 14 to 54. the mean serum pre-dose concentration of infliximab was gradually decreased until Week 14 in the IV 3 mg/kg treatment arm at an interval of 8 weeks, and then a constant concentration was maintained from Week 14 to 30. A pharmacokinetic profile was shown differently until Week 30 due to a difference of dosage form and dosing interval between infliximab IV 3 mg/kg and infliximab SC 120 mg. However, a mean serum concentrations was increased (Weeks 30 to 46) after the IV 3 mg/kg treatment arm was switched to the SC 120 mg at Week 30, and the concentration at Week 54 was similar between respective treatment arms (FIG. 6).

The pharmacokinetic evaluation parameter (AUC_{τ}, Cₘₐₓ and C_{trough}) of Study 3.5 Part 2 for CT-P13 were predicted by using a population PK model. The Cₘₐₓ and C_{trough} showed a flatter profile in the SC 120 mg treatment arm than in the IV 3 mg/kg treatment arm from Week 22 to 30, and it was measured that a predicted C_{trough} of the SC 120 mg treatment arm is higher than a target therapeutic serum concentration, i.e., 1 μg/ml (Table 25).

TABLE 25

| | | |
|---|---|---|
| Parameter | | |
| Visit | IV 3 mg/kg | SC 120 mg |
| Statistics | (N = 174) | (N = 166) |

AUC_{τ} (hr*μg/ml)

| | | |
|---|---|---|
| Week 22 | | |
| Number of Patients | 165 | 162 |
| Mean (CV %) | 14156.9 (46.3) | 5311.5 (45.6) |
| Week 24 | | |
| Number of Patients | — | 160 |
| Mean (CV %) | — | 5187.9 (45.3) |
| Week 26 | | |
| Number of Patients | — | 161 |
| Mean (CV %) | — | 5273.1 (47.3) |
| Week 28 | | |
| Number of Patients | — | 160 |
| Mean (CV %) | — | 5157.2 (46.6) |

Cmax (μg/ml)

| | | |
|---|---|---|
| Week 22 | | |
| Number of Patients | 165 | 162 |
| Mean (CV %) | 71.597 (16.890) | 17.744 (40.868) |
| Week 24 | | |
| Number of Patients | — | 160 |
| Mean (CV %) | — | 17.623 (40.732) |
| Week 26 | | |
| Number of Patients | — | 161 |
| Mean (CV %) | — | 17.633 (41.102) |
| Week 28 | | |
| Number of Patients | — | 160 |
| Mean (CV %) | — | 17.539 (40.631) |

Predicted C_{trough} (μg/ml)

| | | |
|---|---|---|
| Week 22 | | |
| Number of Patients | 165 | 162 |
| Mean (CV %) | 1.486 (168.413) | 12.185 (54.246) |
| Week 24 | | |
| Number of Patients | — | 160 |
| Mean (CV %) | — | 12.303 (53.957) |
| Week 26 | | |
| Number of Patients | — | 161 |
| Mean (CV %) | — | 12.181 (53.421) |
| Week 28 | | |
| Number of Patients | — | 160 |
| Mean (CV %) | — | 12.165 (54.582) |

* Abbreviation: AUC_{τ}: Model predicted area under the concentration-time curve at steady state (Weeks 22-30), Cₘₐₓ: Model predicted maximum serum concentration, C_{trough}: Model predicted trough serum concentration, and CV %: Percent coefficient of variation.
** Patients from IV 3 mg/kg treatment arm were administered the infliximab every 8 weeks, while patients from SC 120 mg treatment arm were administered the infliximab every 2 weeks. Thus, the PK parameters of IV 3 mg/kg treatment arm were obtained at Week 22 and those of the SC 120 mg treatment arm were obtained for Weeks 22, 24, 26 and 28. Thus, the pharmacokinetic parameters of the IV 3 mg/kg treatment arm were assessed for Week 22 and those of the SC 120 mg treatment arm were assessed for Weeks 22, 24, 26 and 28.

### Example 3. Evaluation of Efficacy and Safety on Subcutaneous Injection of Infliximab as Maintenance Therapy for Crohn's Disease (CD) Patients (Study 3.8)

### Example 3-1. Study Protocol

The present study was a randomized, placebo-controlled, double-blind, multi-center, parallel-group and phase III trial designed to evaluate the efficacy, PK, PD, utility and safety of the infliximab (CT-P13) SC.

The present study was composed of three study periods: Screening, Treatment Period (induction, maintenance and extension phases), and End-of-Study visit.

Screening period: The Screening was carried out between Days −42 and 0 (the maximum 6 weeks) before an initial administration of the infliximab IV to be administered during the induction phase.

Patients had to meet all the following inclusion criteria to be enrolled in this study.
Patient who was male or female aged between 18 and 75;
Patient who had moderately to severely active CD with a CDAI score of 220-450 points;
Patient who had ileal-colonic CD with a simplified endoscopic activity score for CD of 6 points or higher, or who had ileal or colonic CD with such score of 4 points or higher as well as an ulcer score for at least one compartment;
Patient who had been diagnosed as CD through radiographic inspection, biopsy or endoscopy at least three months before the first day of administration of the study drug;
Patient who had received an adequate treatment for active CD with a corticosteroid and/or an immunosuppressant, but had not responded to, or who had no drug tolerance to or had medical contraindications for such therapy.

Patients meeting any of the following criteria were excluded from this study.
Patient who had previously received 2 or more biologic agents, 2 or more Janus kinase (JAK) inhibitors, or 2 or more of both biologic agents and JAK inhibitors;
Patient who used a TNFα inhibitor or a biological agent within five half-lives based on an initial administration of the study drug (Day 0);
Patient who had not previously responded to or had no drug tolerance to a TNFα inhibitor, which was used for treatment of CD; and
Patient who had previously used infliximab for treatment of CD or other diseases.

Treatment Period:
Open induction phase (administered at Weeks 0, 2 and 6)
Double-blind maintenance phase (administered from Week 10 to 54)
Open extension phase (administered from Week 56 to 102)

In the open induction phase, only the patients who met all the selection criteria and did not correspond to any of the exclusion criteria based on Day 0 (Week 0) were enrolled into the study. All the patients enrolled paid a visit at Weeks 0, 2 and 6 for induction treatment and were dosed with the infliximab IV (5 mg/kg) for two hours. Out of patients who received a full dose of infliximab three times via IV infusion, those who were classified as patients responded at Week 10 according to CDAI-100 and had no concerns about safety according to the investigator's decision were randomly assigned to an infliximab SC group or a placebo SC before treatment on Day 70 (Week 10).

Such random assignment with regard to administration of the study drug was stratified according to the following criteria:
Previous use of a biological agent and/or a JAK inhibitor (use or non-use);
Use of treatment with oral corticosteroids at Week 0 (use or non-use); and
Achievement of clinical remission at Week 10 (remission achieved or not achieved by CDAI scores).

The double-blind maintenance phase was composed of an additional dose of the infliximab SC or the placebo SC, in which a last dose was administered at Week 54.
Test group 1) infliximab SC 120 mg every two weeks: Dosed with the infliximab SC 120 mg via PFS every 2 weeks from Week 10 to 54.
Test group 2) Placebo SC every two weeks: Dosed with the placebo SC via PFS every 2 weeks from Week 10 to 54.

In the open extension phase, the maintenance phase was completed until Week 54, and patients, who were deemed to benefit from continuous treatment according to the investigator's opinion, were dosed with the infliximab SC 120 mg via PFS or AI. Patients who were dosed with the infliximab SC 240 mg in the maintenance phase received the same dose thereof in the extension phase. The extension phase was continued until Week 102.

If patients initially responded to the drug regardless of assigned groups but lost the response after then, they were allowed to have a dose adjusted to an administration of the infliximab SC 240 mg (double injections of the infliximab SC 120 mg (twice)) every two weeks starting from Week 22. The loss of response was defined as an increase in CDAI of 100 points from the Week 10 CDAI score with a total score ≥220.

Patients might receive premedication 30 to 60 minutes before the administration of the infliximab IV, and might be dosed with, but are not limited to, antihistamine (at an equivalent dose of 2-4 mg of chlorpheniramine), hydrocortisone, paracetamol and/or non-sedating antihistamine (at an equivalent dose of 10 mg of cetirizine) at the investigator's discretion during the clinical period. Patients might receive premedication at the investigator's discretion even during the administration of the infliximab SC.

End-of-Study visit: In four weeks after the last administration, a final visit for finishing the study was performed. For patients who discontinued the study treatment early on before being switched to the infliximab SC or the placebo SC at Week 10, the End-of-Study visit was performed in eight weeks after the last infliximab IV was administered.

### Example 3-2. PK Data and PK-PD Modeling for CD Patients

The administered dose and interval of the CT-P13 used in Study 3.8 were determined based on the results of Study 1.6 Part 1 for CT-P13 and the results of the population PK analysis. A PK-PD model was based on the CT-P13 IV administration data on healthy volunteers, patients with AS, patients with RA and patients with CD, as well as the infliximab SC administration data on patients with CD, patients with RA and healthy volunteers (Clinicaltrials.gov Identifier Code NCT01220518 (Study 1.1), NCT01217086 (Study 3.1), NCT02096861 (Study 3.4), NCT03147248 (Study 3.5) and NCT02883452 (Study 1.6)).

The PK-PD model developed based on the data above might be used to simulate the SC administration results for patients having the indications of infliximab (RA, UC, CD, plaque psoriasis, psoriatic arthritis or AS).

In case of the population PK and PK-PD modeling for CD patients, not only the safety for indications (CD, RA and AS) but also a total of CDAI scores were included in the analysis. The final PK model was performed by a linear elimination from the central compartment in which infliximab infusion occurs, and a 2-compartment model having a depot compartment with a first-order absorption rate toward a central compartment. A covariance relationship between disease duration time and baseline CDAI score was applied to the model. The PK-PD modeling verification was performed through visual predictive checks (VPC).

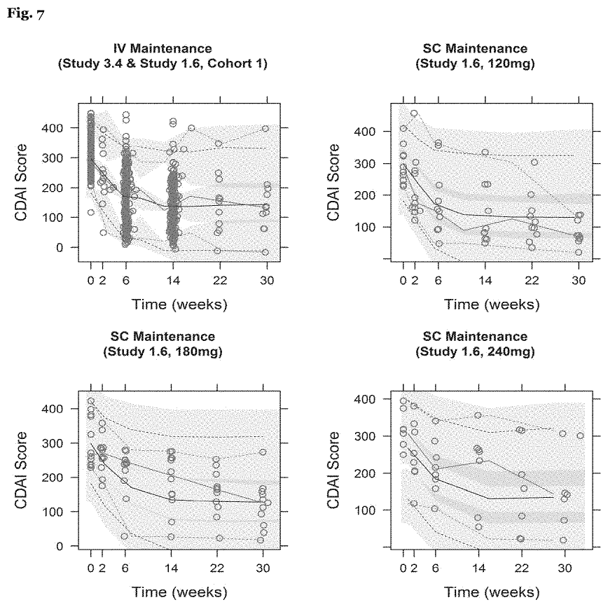

FIG. 7 is a graph comparing CDAI scores observed (indicated by ∘) and model-predicted CDAI scores (black solid line) with each other as a result of VPC obtained from a final PK-PD model. The data of Study 1.6 Part 1 for CT-P13 were limited, but it was identified from the VPC that there is a fair degree of agreement between the observed data and the simulated data as shown in the results of FIG. 7.

### Example 3-3. Exposure-Response Assessment on Efficacy from CD Patients

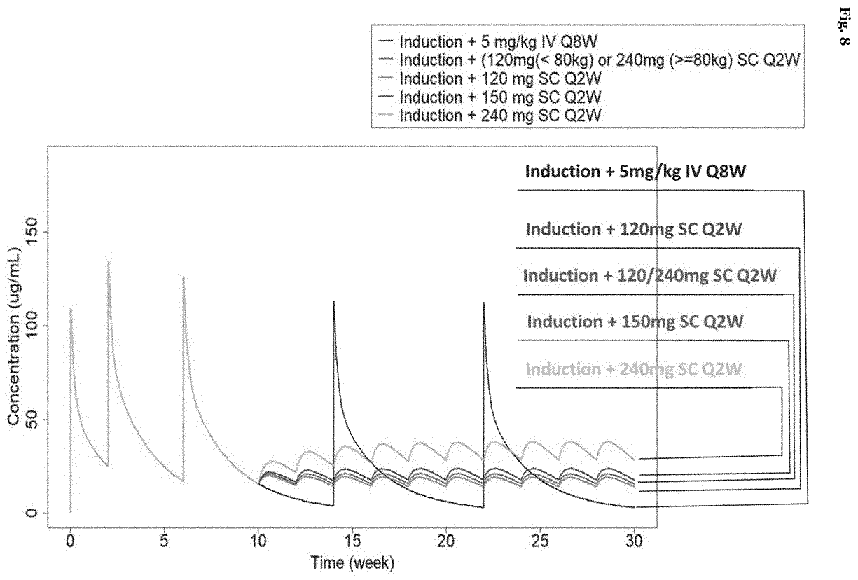

An average serum concentration with an elapse of time was simulated with regard to various doses (120, 150 and 240 mg) of infliximab SC as shown in FIG. 8. All the simulated infliximab SC doses maintained a continuously higher level of C_{trough} than that of an IV reference drug from Week 10 to 30, which agreed with the results of Study 1.6 Part 1 for infliximab.

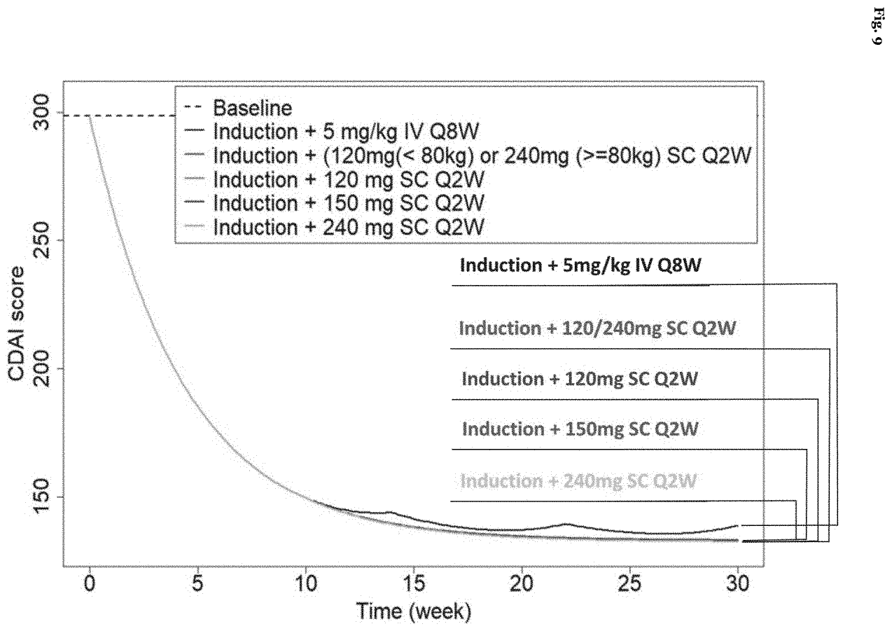

Further, it was identified from FIG. 9 that no particular difference is expected between the predicted CDAI scores after the administration of infliximab SC at different doses.

Based on the simulation results, the three SC doses of 120, 150 and 240 mg all maintained a continuously high level of C_{trough} from Week 10 to 30, and thus all the doses are determined as a optimal dose. Out of those doses, it was demonstrated that the most efficient dose for achieving a desired level of efficacy with the least drug exposure is 120 mg. In addition, in Study 1.6 Part 1 for infliximab, no safety problem was observed from the infliximab SC 120 mg cohort, and the number of patients with positive results for anti-drug antibodies (ADA) or neutralizing antibodies (NAb) was lowest in the infliximab SC 120 mg cohort. Accordingly, the present inventors propose a method for administering infliximab 120 mg every 2 weeks from Week 10 for a follow-up Study 3.8 for infliximab.

### Example 3-4. Basis for Time Point of First Subcutaneous Administration (Week 10)

In the proposed administration method, the administration method during the IV induction phase was the same as the conventionally approved one of infliximab. IV administration was performed at a dose of 5 mg/kg over 2 hours or so at Weeks 0, 2 and 6. After that, SC administration commenced at Week 10, i.e., in 4 weeks after the administration of the last IV induction dose at Week 6. At the time point of starting the first SC administration, a level of C_{trough} was to be maintained close to the plasmatic concentration at steady state throughout the SC administration therapy.

Based on the PK modeling data, simulation was performed to find an optimal time point for performing the first SC administration. According to the results of simulating the profile of plasmatic concentrations of infliximab (±SD), the plasmatic concentration at Week 10, i.e., in 4 weeks after the last IV induction was arranged closet to an average plasmatic concentration after three IV induction administrations at Weeks 0, 2 and 6. It might be identified that C_{trough} at steady state is expected during the maintenance phase of infliximab SC and show less fluctuation of PK concentrations, when administered SC at Week 10 and subsequently at an interval of two weeks (See FIG. 8). Thus, it was demonstrated that a predicted average C_{trough} would be better maintained throughout the study and quickly reach a steady state, if SC administration commences at Week 10.

Consequently, it was identified from the results of PK-PD modeling and simulation that all the administration therapies of CT-P13 SC achieve an adequate efficacy from CD patients without any unexpected safety signal. The simulation was successfully used to identify an optimal administration therapy of infliximab SC that may be applied to CD patient from now on.

From a follow-up simulation study on CD patients, a method for administering 120 mg of infliximab SC every 2 weeks seems to be most suitable. Accordingly, the present inventors propose an IV induction dose of 5 mg/kg at Weeks 0, 2 and 6, and then propose an SC maintenance therapy of 120 mg every two weeks after starting an SC administration at Week 10.

### Example 4. Evaluation of Efficacy and Safety on Subcutaneous Injection of Infliximab as Maintenance Therapy for Ulcerative Colitis (UC) Patients (Study 3.7)

### Example 4-1. Study Protocol

The present study was a randomized, placebo-controlled, double-blind, multi-center, parallel-group and phase III trial designed to evaluate the efficacy, PK, PD and safety of the infliximab SC.

The present study was composed of three study periods: Screening, Treatment Period (induction, maintenance and extension phases), and End-of-Study visit.

Screening period: The Screening was carried out between Days −42 and 0 (the maximum 6 weeks) before an initial administration of the infliximab IV to be administered during the induction phase.

Patients had to meet all the following inclusion criteria to be enrolled in this study.
Patient who was male or female aged between 18 and 75;
Patient who had moderately to severely active UC with a modified Mayo score of 5-9 points;
Patient who had been diagnosed as UC through endoscopy or radiographic inspection and biopsy; and
Patient who had been treated for active UC, but had not responded to universal therapeutic agents such as corticosteroids and/or 6-mercaptopurine, azathioprine or the like; or who had no drug tolerance to or had medical contraindications for such therapies.

Patients meeting any of the following criteria were excluded from this study.
Patient who had previously used two or more biological agents or two or more Janus kinase (JAK) inhibitors, or who had previously used two or more of biological agents and JAK inhibitors;
Patient whose TNFα inhibitor or biological agent may be detected in serum or may be within five half-lives before an initial administration of the study drug (Day 0);
Patient who had been dosed with a TNFα inhibitor for UC treatment, but had not responded to or had no drug tolerance to such therapy; or
Patient who had previously used infliximab for treatment of UC or other diseases.

Treatment Period:
Open induction phase (administered at Weeks 0, 2 and 6)
Double-blind maintenance phase (administered from Week 10 to 54)
Open extension phase (administered from Week 56 to 102) In the open induction phase, only the patients who met all the selection criteria and did not correspond to any of the exclusion criteria based on Day 0 (Week 0) were enrolled into the study. All the patients enrolled paid a visit at Weeks 0, 2 and 6 for induction treatment and were dosed with the infliximab IV (5 mg/kg) for 2 hours. Out of patients who received a full dose of infliximab three times via IV infusion, only patients who were classified as respondents according to Mayo score at week 10 and had no concerns about safety at the investigator's discretion were randomly assigned to receive the infliximab SC or the placebo SC before treatment on Day 70 (Week 10).

Such random assignment with regard to administration of the study drug was stratified according to the following criteria:
Previous use of a biological agent and/or a JAK inhibitor (use or non-use);
Use of treatment with oral corticosteroids at Week 0 (use or non-use); and
Achievement of clinical remission at Week 10 (remission achieved or not achieved by modified Mayo scores).

The double-blind maintenance phase was composed of the infliximab SC or the placebo SC at an additional dose, in which a last dose was administered before Week 54.
Test group 1. Infliximab SC 120 mg every 2 weeks: Dosed with the infliximab SC 120 mg via PFS every 2 weeks from Week 10 to 54.
Test group 2. Placebo SC every 2 weeks: Dosed with the placebo SC via PFS every 2 weeks from Week 10 to 54.

In the open extension phase, the maintenance phase was completed until Week 54, and patients, who were deemed to benefit from continuous treatment according to the investigator's opinion, continued the study until the open extension phase. The administration for the extension phase commenced at Week 56 and continued until Week 102. Patients who were dosed with the infliximab SC 120 mg or the placebo SC also received the infliximab SC 120 mg at Week 54. Patients who were dosed with the infliximab SC 240 mg at Week 54 received the same dose thereof in the extension phase.

If patients initially responded to the drug regardless of assigned groups but lost the response after then, they were allowed to have an increase in dose to the infliximab SC 240 mg (double injections of the infliximab SC 120 mg (twice)) every 2 weeks starting from Week 22. The loss of response was defined as one of the followings: An increase in modified Mayo scores by 2 points or more and 30% or more compared to Week 10; total scores by 5 points or more; and endoscopic scores by 2 points or more.

Patients might receive premedication 30 to 60 minutes before starting the administration of the infliximab IV, and might be dosed with antihistamine (at an equivalent dose of 2-4 mg of chlorpheniramine), hydrocortisone, paracetamol and/or non-sedating antihistamine (at an equivalent dose of 10 mg of cetirizine), but not limited thereto, at the investigator's discretion during the clinical period. Patients might receive premedication at the investigator's discretion even during the administration of subcutaneous injection.

End-of-Study visit: In 4 weeks after the last administration, a final visit for finishing the study was performed. For patients who discontinued the study treatment early on before being switched to the infliximab SC or the placebo SC, the End-of-Study visit was performed in 8 weeks after the last infliximab IV was administered.

### Example 4-2. PK Data and PK-PD Modeling for UC Patients

The administered dose and interval of the CT-P13 used in Study 3.7 were determined based on the results of Study 1.6 Part 1 for CT-P13. A PK-PD model was based on the CT-P13 IV administration data on healthy volunteers, patients with AS, patients with RA and patients with CD, as well as the infliximab SC administration data on patients with CD, patients with RA and healthy volunteers (Clinicaltrials.gov Identifier Code NCT01220518 (Study 1.1), NCT01217086 (Study 3.1) and NCT02096861 (Study 3.4)).

The PK-PD model developed based on the data above might be used to simulate the SC administration results for patients having the indications of infliximab (RA, UC, CD, plaque psoriasis, psoriatic arthritis or AS).

In case of the population PK and PK-PD modeling for UC patients, not only the safety for indications (CD, RA and AS) but also a Mayo scores were included in the analysis. The final PK model was performed by a linear elimination from the central compartment in which infliximab infusion occurs, and a 2-compartment model having a depot compartment with a first-order absorption rate forward a central compartment. In the final PD model, a covariance relationship between disease duration time and baseline Mayo score was applied to the model.

### Example 4-3. Exposure-Response Assessment on Efficacy from UC Patient Data

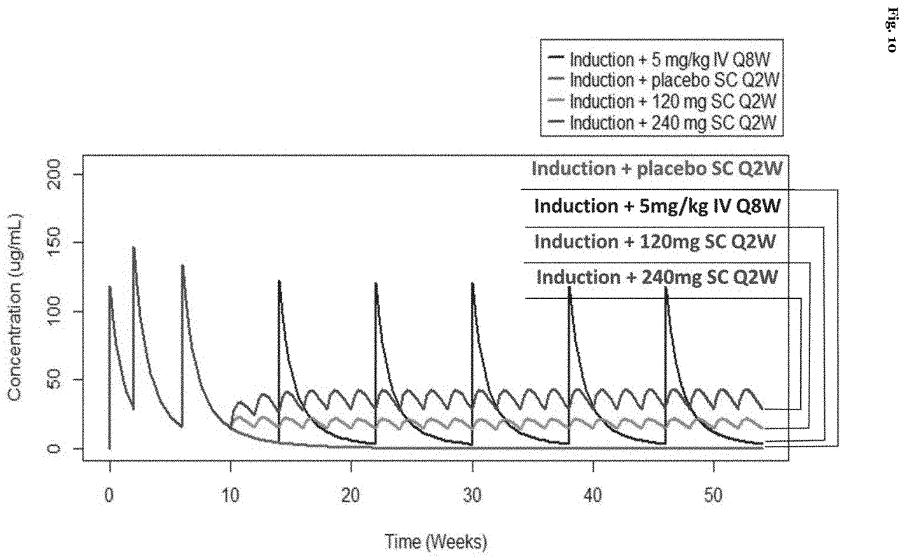

An average serum concentration with an elapse of time was simulated with regard to various doses (120 and 240 mg) of CY-P13 SC as shown in FIG. 10. All the simulated infliximab SC doses maintained a continuously higher level of C_{trough} from Week 10 to 30 than that of an IV reference drug, which agreed with the results of Study 1.6 Parts 1 and 2 for infliximab.

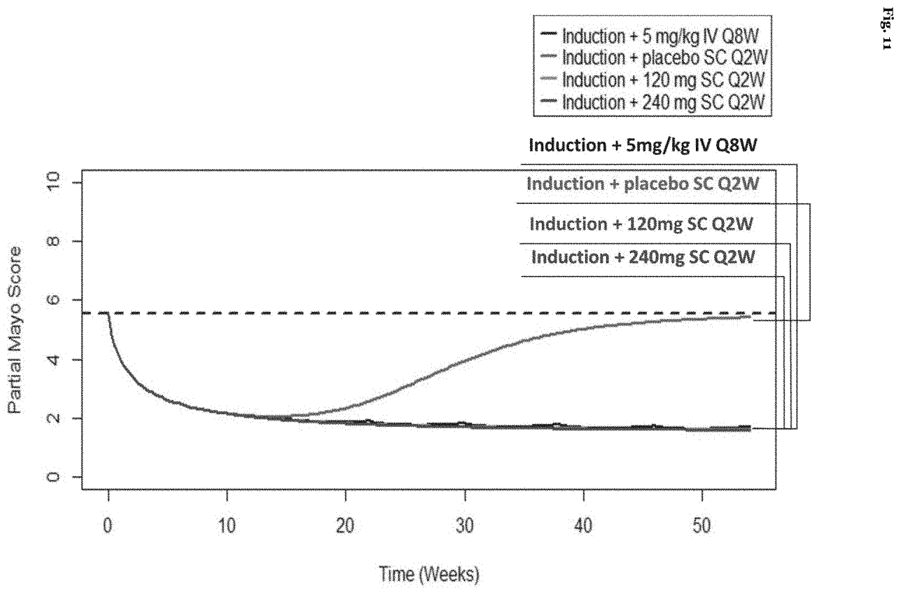

Further, it was expected from FIG. 11 that there is no particular difference between Mayo scores predicted after the administration of infliximab SC at different doses, and it might be identified that there is a similar effect in 120 and 240 mg all.

Based on the simulation results, the SC doses of 120 and 240 mg all maintained a continuously high level of C_{trough} including a steady state from Week 10 to 54, and thus both doses are determined as a optimal dose. Out of those doses, it was identified that the most efficient dose for achieving a desired level of efficacy with the least drug exposure is 120 mg. In addition, in Study 1.6 Part 1 for infliximab, no safety issue was observed from the infliximab SC 120 mg cohort, and the number of patients with positive results for anti-drug antibodies (ADA) or neutralizing antibodies (Nab) was lowest in the infliximab SC 120 mg cohort. Accordingly, the present inventors propose a method for administering infliximab 120 mg every 2 weeks from Week 10 for a follow-up CT-P13 3.7 study.

1. A method for treating TNFα-related diseases, the method comprising:
a step of administering to a patient a pharmaceutical composition comprising an anti-TNFα antibody or an antigen-binding fragment thereof,
wherein an anti-TNFα antibody or an antigen-binding fragment thereof is subcutaneously administered to a patient at a dose of 60 to 300 mg and at intervals of 1 to 8 weeks.

2. The method according to claim 1, wherein the TNFα-related diseases are selected from the group consisting of rheumatoid arthritis, ulcerative colitis, Crohn's disease, plaque psoriasis, psoriatic arthritis and ankylosing spondylitis.

3. The method according to claim 1, wherein the anti-TNFα antibody or the antigen-binding fragment thereof is administered to a patient at a dose of 80 to 100 mg, 110 to 130 mg, 170 to 190 mg, or 230 to 250 mg.

4. The method according to claim 1, wherein the anti-TNFα antibody or the antigen-binding fragment thereof is administered at an increased dose, when the patient's condition is not improved or therapeutic response is lost.

5. The method according to claim 2, wherein the TNFα-related disease is rheumatoid arthritis.

6. The method according to claim 5, wherein the anti-TNFα antibody or the antigen-binding fragment thereof is administered to the patient at a dose of 90 to 180 mg.

7. The method according to claim 6, wherein the anti-TNFα antibody or the antigen-binding fragment thereof is administered to the patient at a dose of 90, 120 or 180 mg.

8. The method according to claim 2, wherein the TNFα-related diseases are selected from the group consisting of ulcerative colitis, Crohn's disease, plaque psoriasis, psoriatic arthritis and ankylosing spondylitis.

9. The method according to claim 8, wherein the anti-TNFα antibody or the antigen-binding fragment thereof is administered to the patient at a dose of 120 to 240 mg.

10. The method according to claim 9, wherein the anti-TNFα antibody or the antigen-binding fragment thereof is administered to the patient at a dose of 120, 150, 180 or 240 mg.

11. The method according to claim 1, wherein the anti-TNFα antibody or the antigen-binding fragment thereof is administered to a patient at intervals of 1, 2, 3, 4, 5, 6, 7 or 8 weeks.

12. The method according to claim 11, wherein the anti-TNFα antibody or the antigen-binding fragment thereof is administered to the patient at intervals of 2 or 4 weeks.

13. The method according to claim 1, wherein the anti-TNFα antibody or the antigen-binding fragment thereof is administered in combination with one or more selected from the group consisting of disease-modifying anti-rheumatic drugs (DMARDs), steroids and immunosuppressants.

14. The method according to claim 13,
wherein the disease-modifying anti-rheumatic drugs (DMARDs) are selected from the group consisting of methotrexate, leflunomide, sulfasalazine and hydroxychloroquine,
wherein the steroids are selected from the group consisting of corticosteroid, glucocorticoid, cortisol, mineralocorticoid and aldosterone, and
wherein the immunosuppressants are selected from the group consisting of azathioprine, 6-mercaptopurine, cyclosporin A, tacrolimus, mycophenolic acid, bredinin, mTOR inhibitor and anti-lymphocyte antibody.

15. The method according to claim 1, wherein the patient is a patient who has been intravenously administered with the anti-TNFα antibody or the antigen-binding fragment thereof at least once prior to subcutaneous administration.

16. The method according to claim 15, wherein the patient is a patient who has been intravenously administered with the anti-TNFα antibody or the antigen-binding fragment thereof 2 or 3 times prior to subcutaneous administration.

17. The method according to claim 15, wherein
a) the patient who has a rheumatoid arthritis disease is a patient who has been intravenously administered with the anti-TNFα antibody or the antigen-binding fragment thereof 2 times prior to subcutaneous administration, and
b) the patient who has one or more diseases selected from the group consisting of ulcerative colitis, Crohn's disease, plaque psoriasis, psoriatic arthritis and ankylosing spondylitis is a patient who has been intravenously administered with the anti-TNFα antibody or the antigen-binding fragment thereof 2 or 3 times prior to subcutaneous administration.

18. The method according to claim 15, wherein the patient is a patient who has been intravenously administered with the anti-TNFα antibody or the antigen-binding fragment thereof twice at Weeks 0 and 2, or a patient who has been intravenously administered with the same 3 times at Weeks 0, 2 and 6 prior to subcutaneous administration.

19. The method according to claim 15, wherein the patient is a patient who has been intravenously administered with the anti-TNFα antibody or the antigen-binding fragment thereof at a dose of 1 to 10 mg/kg per administration.

20. The method according to claim 19, wherein the patient is a patient who has been intravenously administered with the anti-TNFα antibody or the antigen-binding fragment thereof at a dose of 3 to 5 mg/kg per administration.

21. The method according to claim 20, wherein
a) the patient who has a rheumatoid arthritis disease is a patient who has been intravenously administered with the anti-TNFα antibody or the antigen-binding fragment thereof at a dose of 3 mg/kg per administration; and
b) the patient who has one or more diseases selected from the group consisting of ulcerative colitis, Crohn's disease, plaque psoriasis, psoriatic arthritis and ankylosing spondylitis is a patient who has been intravenously administered with the anti-TNFα antibody or the antigen-binding fragment thereof at a dose of 5 mg/kg per administration.

22. The method according to claim 15, wherein the first subcutaneous administration is performed in 2 to 8 weeks after the last intravenous administration.

23. The method according to claim 22, wherein the first subcutaneous administration is performed in 4 weeks after the last intravenous administration.

24. The method of claim 1, wherein a minimum serum concentration (C_{trough}) of the anti-TNFα antibody or the antigen-binding fragment thereof is maintained at 0.01 μg/ml or more after being subcutaneously administered to the patient.

25. The method according to claim 24, wherein
a) the minimum serum concentration (C_{trough}) of the anti-TNFα antibody or the antigen-binding fragment thereof is maintained at 1 μg/ml or more for the patient with a rheumatoid arthritis disease; and
b) the minimum serum concentration (C_{trough}) of the anti-TNFα antibody or the antigen-binding fragment thereof is maintained at 5 μg/ml or more for the patient who has one or more diseases selected from the group consisting of ulcerative colitis, Crohn's disease, plaque psoriasis, psoriatic arthritis and ankylosing spondylitis.

26. The method according to claim 1, wherein the patient after the subcutaneous administration has one or more of the following characteristics:
a) a decrease in DAS28 (Disease Activity Score in 28 joints) by at least 2.0; or
b) a decrease in CDAI (Crohn's disease activity index) by at least 70.

27. The method according to claim 1, wherein the patient before the subcutaneous administration has one or more of the following characteristics:
a) Having an inadequate response to disease-modifying anti-rheumatic drugs (DMARDs) comprising methotrexate;
b) Not having previously been treated with methotrexate and other DMARDs;
c) Exhibiting a rise in serologic indicators associated with severe axial symptoms and inflammation, which show no proper response to common therapies; or
d) Not responding to, being contraindicated from, or having intolerance to methotrexate, cyclosporine, or systemic therapies comprising dermatologic photochemotherapy (psoralen ultraviolet A therapy: PUVA).

28. The method according to claim 1, wherein the patient before subcutaneous administration has one or more of the following characteristics:
a) having no adequate response to, having intolerance to, or being contraindicated from treatment with corticosteroids, 6-mercaptopurine, azathioprine or immunosuppressants; or
b) Not responding to common therapies, comprising antibiotic, excretion or immunosuppressive therapies.

29. The method according to claim 1, wherein the anti-TNFα antibody or the antigen-binding fragment thereof comprises:
a light-chain variable region comprising a CDR1 domain comprising an amino acid sequence of SEQ ID NO: 1, a CDR2 domain comprising an amino acid sequence of SEQ ID NO: 2, and a CDR3 domain comprising an amino acid sequence of SEQ ID NO: 3; and
a heavy-chain variable region comprising a CDR1 domain comprising an amino acid sequence of SEQ ID NO: 4, a CDR2 domain comprising an amino acid sequence of SEQ ID NO: 5, and a CDR3 domain comprising an amino acid sequence of SEQ ID NO: 6.

30. The method according to claim 1, wherein the anti-TNFα antibody is infliximab.

31. The method according to claim 1, wherein the composition comprising the anti-TNFα antibody or the antigen-binding fragment thereof comprises: (A) 90 to 180 mg/ml of the anti-TNFα antibody or the antigen-binding fragment thereof; (B) 0.02 to 0.1% (w/v) of polysorbate; (C) 1 to 10% (w/v) of sorbitol; and (D) 1 to 50 mM of a buffer comprising acetate.

32. The method according to claim 1, wherein the composition comprising the anti-TNFα antibody or the antigen-binding fragment thereof is filled into a pre-filled syringe or an auto-injector before being administered to the patient.

33. A pharmaceutical composition for treating TNFα-related diseases comprising an anti-TNFα antibody or an antigen-binding fragment thereof, wherein the anti-TNFα antibody or the antigen-binding fragment thereof is subcutaneously administered at a dose of 60 to 300 mg and at intervals of 1 to 8 weeks.

34. A kit comprising:
(a) a pharmaceutical composition comprising an anti-TNFα antibody or an antigen-binding fragment thereof; and
(b) instructions that direct the anti-TNFα antibody or the antigen-binding fragment thereof to be subcutaneously administered at a dose of 60 to 300 mg and at intervals of 1 to 8 weeks in order to treat a patient having a TNFα-related disease.

35. A use of an anti-TNFα antibody or an antigen-binding fragment thereof in preparation of a pharmaceutical composition to be administered to a patient in order to treat a TNFα-related disease, wherein the anti-TNFα antibody or the antigen-binding fragment thereof is to be subcutaneously administered at a dose of 60 to 300 mg and at intervals of 1 to 8 weeks.
